(19) United States
(12) Patent Application Publication
(10) Pub. No.: US 2022/0251146 A1
(43) Pub. Date: Aug. 11, 2022

(54) DNA-DEGRADING PROTEINS AND USES THEREOF

(71) Applicant: President and Fellows of Harvard College, Cambridge, MA (US)

(72) Inventors: Karine Gibbs, Cambridge, MA (US); Denise Sirial, Cambridge, MA (US)

(73) Assignee: President and Fellows of Harvard College, Cambridge, MA (US)

(21) Appl. No.: 17/609,171

(22) PCT Filed: May 6, 2020
(86) PCT No.: PCT/US2020/031686

§ 371 Date: Nov. 5, 2021

Related U.S. Application Data
(60) Provisional application No. 62/843,868, filed on May 6, 2019.

(51) Int. Cl. C07K 14/24; A61P 31/04; A61P 17/02

(52) U.S. Cl. CPC C07K 14/24; C07K 2319/00; A61P 17/02; A61P 31/04

(57) ABSTRACT

The present disclosure provides proteins, protein fragments, protein variants, fusion proteins, nucleic acids, vectors, cells, methods, kits, and compositions for cleaving DNA and/or inhibiting growth of bacterial growth using IdrD protein or a fragment thereof. In some embodiments, the disclosure provides an IdrD protein comprising a fragment of IdrD protein from Proteus mirabilis, a fragment of an IdrD protein from Rothia, or a fusion thereof. In some embodiments, the disclosure provides a method for inhibiting bacterial biofilm formation on a surface, the method comprising contacting or coating the surface with an IdrD protein. In some embodiments, the disclosure provides a method for inhibiting bacterial biofilm formation on a surface, the method comprising contacting or coating the surface with an IdrD protein.

### CROSS REFERENCE TO RELATED APPLICATIONS

This application claims priority under 35 U.S.C. § 119(e) to United States Provisional Application, U.S. Ser. No. 62/843,868, filed May 6, 2019, which is incorporated herein by reference.

### BACKGROUND OF THE INVENTION

Multiple manifestations of human disease (e.g., infections, cancer) are characterized by having cells (e.g., bacterial cells and/or human cells) embedded within a complex matrix of polysaccharides and/or DNA. Current medical treatments generally involve manually removing (e.g., debridement) or chemically shrinking or destroying the matrix (e.g., DNase I, restriction enzymes) in combination with administering anti-bacterial or anti-neoplastic drugs. However, destruction of extracellular DNA may be sufficient to disrupt the matrix. Accordingly, DNA-degrading proteins may be therapeutically effective in treating a wide range of human diseases involving a complex matrix of cells, polysaccharides, and DNA, e.g., infections, cancer, wounds, lung disease (e.g., cystic fibrosis), and vascular disease (e.g., thrombosis).

### SUMMARY OF THE INVENTION

The present disclosure is based on the discovery that a fragment of IdrD protein from Proteus mirabilis degrades DNA in vitro, and that the DNA-degrading activity of IdrD affects the viability, swarming, and spatial distribution of P. mirabilis. Several features of the fragment of IdrD protein provide improvements compared to known DNA-degrading proteins, including degradation of DNA in various forms (e.g., lambda DNA, linear DNA, and supercoiled DNA), highly efficient degradation by small quantities of IdrD protein (e.g., nanogram (ng) quantities of a fragment of IdrD protein), and efficient delivery of IdrD protein due to its small size (e.g., ˜17 kDa fragment of IdrD protein).

Thus, some embodiments of the present disclosure provide an IdrD protein having DNA-degrading activity. In some embodiments, the IdrD protein comprises a fragment of IdrD protein from P. mirabilis (e.g., SEQ ID NO: 1). In some embodiments, the IdrD protein comprises a fragment of IdrD protein from Rothia (e.g., SEQ ID NO: 5). IdrD proteins as described herein encompass fusions of the fragment of IdrD protein from P. mirabilis and the fragment of IdrD protein from Rothia.

In some embodiments, the present disclosure provides an expression vector comprising a nucleic acid encoding an IdrD protein as described herein. In some embodiments, the present disclosure provides a host cell comprising an expression vector comprising a nucleic acid encoding an IdrD proteins as described herein. In some embodiments, the present disclosure provides a pharmaceutical composition comprising an IdrD protein as described herein.

The present disclosure provides, in some embodiments, methods comprising administering an IdrD protein described herein. In some embodiments, the present disclosure provides methods for inhibiting bacterial growth on a surface comprising contacting or coating the surface with an IdrD protein as described herein. In some embodiments, the present disclosure provides methods for treating a disease associated with bacteria trapping neutrophil extracellular traps (NETs) comprising administering to a subject in need thereof a therapeutically effective amount of an IdrD protein as described herein. In some embodiments, the present disclosure provides methods of treating or preventing a bacterial infection comprising administering to a subject in need thereof a therapeutically effective amount of an IdrD protein as described herein. In some embodiments, the present disclosure provides methods for cleaving DNA comprising incubating a sample comprising DNA and an effective amount of an IdrD protein as described herein.

These and other aspects and embodiments of the invention will be described in greater detail herein. The description of exemplary embodiments of IdrD protein is provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1A shows a schematic depiction of full-length IdrD protein and IdrD-CT, both drawn to scale. Amino acid number are indicated along the bottom, and boxes denote PAAR (SEQ ID NO: 24) and Rhs domains in the N-terminal region. Predicted secondary structure of IdrD-CT is shown with catalytic residues labeled within PD-(D/E)XK domain (underlined).

FIG. 1B shows results from an assay in which viable P. mirabilis cells were measured after overexpression of IdrD-CT or IdrD-CT containing mutations in the predicted catalytic core as compared to the negative control for protein production (GFP).

FIG. 1C shows a schematic of an in vitro DNase assay, in accordance with some embodiments of the technology described herein. IdrD-CT-FLAG and IdrD-CT_{D39A}-FLAG were produced in a cell-free system. A DNA substrate was added to the reaction mixture to assay for nuclease activity.

FIG. 1D shows a α-FLAG western blot with gradient of known concentrations of FLAG-tagged E. coli bacterial alkaline phosphatase (FLAG-BAP) used to estimate amount of protein produced by the cell-free system.

FIG. 1E shows an agarose gel of increasing amounts of IdrD-CT-FLAG and IdrD-CTD_{39A}-FLAG (2.5, 5 and 10 ng) added to methylated and unmethylated lambda DNA (48,502 bp). “Ladder” is the NEB 2-log DNA ladder. DNA is not visible after incubation with 10 ng of IdrD-CT-FLAG.

FIG. 1F shows micrographs of P. mirabilis grown on a swarm-permissive surface. Row “IdrD-CT” is P. mirabilis overproducing the wild-type protein from a plasmid. The negative control is P. mirabilis with an equivalent empty vector; healthy P. mirabilis cells elongate in swarms. Left, Phase. Right, fluorescence of DAPI-stained DNA. There is a loss of DNA integrity when IdrD-CT is produced.

FIG. 1G shows micrographs of E. coli cells, containing different plasmids for protein expression, isolated from mid-logarithmic growth in LB plus kanamycin. Top, the parent plasmid as a negative control. Middle, wild-type IdrD-CT of P. mirabilis strain BB2000, produced from a plasmid. Bottom, the null mutant IdrD-CT_{D39A} of P. mirabilis strain BB2000, produced from a plasmid. Left, Phase. Right, fluorescence of DAPI-stained DNA. The fluorescence pattern in the middle panel show a lack of dsDNA integrity when IdrD-CT is produced.

FIG. 2A shows a schematic of an alignment of IdrD-CT and similar CTs in three Rothia species (MUSCLE followed by Ali2D on MPI Bioinformatics toolkit) [30]. Predicted alpha helices are boxed and beta sheets are underlined; intensity of shading indicates higher confidence.

FIG. 2B shows a schematic of in vitro DNase assay, in accordance with some embodiments of the technology described herein. The Rothia IdrD-CT variant proteins were produced in a cell-free system and then used to assay nuclease activity.

FIG. 2C shows an agarose of increasing concentrations of ^{Rothia}IdrD-CT-FLAG and ^{Rothia}IdrD-CT_{D39A}-FLAG (2.5, 5 and 10 ng) that were added to methylated and unmethylated lambda DNA (48,502 bp). “Ladder” is the NEB 2-log DNA ladder. No DNA is visible after incubation with ^{Rothia}IdrD-CT-FLAG.

FIG. 2D shows results from N- and C-terminal truncations in P. mirabilis IdrD-CT-FLAG assayed for DNA degradation capability. The IdrD-CT proteins are modular, containing independent domains for enzyme activity and specificity. The figure shows in vitro degradation assays with mutant versions of the ^{Proteus}IdrD-CT protein, none of which lead to DNA degradation. Both domains are essential for DNA degradation.

FIG. 2E shows results from in vitro degradation assays with ^{Proteus}IdrD-CT-FLAG, ^{Rothia}IdrD-CT-FLAG, and engineered inter-genera hybrid IdrD-CT-FLAG proteins. hybrid 1, N-terminal sequence from ^{Proteus}IdrD-CT, C-terminal sequence from ^{Rothia}IdrD-CT; hybrid 2, N-terminal sequence from ^{Rothia}IdrD-CT, C-terminal sequence from ^{Proteus}IdrD-CT. All enzymes lead to DNA degradation.

FIG. 2F shows the gel of FIG. 2E with RNA bands visible. “Ladder” is the NEB 2-log DNA ladder.

FIG. 2G shows the results from an in vitro degradation assay using a single amino acid change (aspartic acid, D, to alanine, A) in D (^{Proteus}IdrD-CT-FLAG) and R (^{Rothia}IdrD-CT-FLAG) based on the predicted active site. Increasing concentrations of protein of ˜2.5, 5, and 10 ng of protein (as denoted by the triangles) to methylated lambda DNA. The control reactions (to match the recombinant proteins) are as follows: with no enzyme is labeled “-” and with no reaction components “Lambda DNA.” No DNA is degraded in the control reactions as evidenced by the bright band in each lane. ^{Proteus}IdrD-CT-FLAG and ^{Rothia}IdrD-CT-FLAG degrade DNA as evidenced by the bright band in each lane by the absence of a bright band in a lane. D* (^{Proteus}IdrD-CT_{D39A}-FLAG) and R* (^{Rothia}IdrD-CT_{D39A}-FLAG), which are single amino acid change, no longer degrades DNA as shown by bright band in each lane.

FIG. 2H shows a Western Blot for protein detection showing epitope-tagged hybrid proteins from FIG. 2E. Both DR (hybrid 1, N-terminal sequence from ^{Proteus}IdrD-CT, C-terminal sequence from ^{Rothia}IdrD-CT) and RD (hybrid 2, N-terminal sequence from ^{Rothia}IdrD-CT, C-terminal sequence from ^{Proteus}IdrD-CT) are stably produced. The ladder (far left lane) contains the Precision Plus Protein™ Kaleidoscope™ prestained protein standards. The arrow marks the expected size (˜17 kDa) for the IdrD-CT and engineered proteins.

FIG. 3A shows a cartoon workflow depicting how short reads from two metagenomes were mapped against a reference containing the four IdrD sequences shown here. Based on the mapping results, each positions' coverage, e.g., the number of metagenomic reads mapping to that position, can be plotted for each metagenome (partially transparent lines; each line is a different metagenome).

FIG. 3B shows coverage of each sequence (subpanel columns) by metagenomes originating from the human gut (top row of subpanels) or human oral cavity (bottom row), for metagenomes covering at least half of that IdrD sequence. The number of metagenomes passing the filter is shown in the top right corner of each subpanel. The positions corresponding to the RHS core and CTD are annotated with dark and light bars, respectively, between the two rows of subpanels.

FIG. 3C shows the x-axis zoomed in to show CTD coverages (region highlighted by the bar in FIG. 3B) by metagenomes covering at least half of that CTD sequence.

FIG. 3D shows coverage for the CTD of three different Prevotella species' IdrD sequences, from metagenomes covering at least half of that CTD sequence.

FIG. 4A shows a graph quantifying viable cells after overexpression of IdrD-CT and active site mutants in liquid-grown E. coli MG1655. Colony-forming units per milliliter over the course of eight hours are plotted on a log₁₀ scale.

FIG. 4B shows the gel of FIG. 1E (^{Proteus}IdrD-CT) with RNA bands visible. “Ladder” is the NEB 2-log DNA ladder. Bands running below 1 kB are presumed to be rRNA and tRNA from PURExpress in vitro translation reactions.

FIG. 4C shows an agarose gel of DNase assays with plasmid DNA as substrates. Agarose gel of in vitro degradation assays using pure protein with plasmid DNA. 10 ng of IdrD-CT-FLAG or the catalytic mutant (IdrD-CT_{D39A}-FLAG), both from Proteus, were independently produced by in vitro translation and incubated with 250 ng of plasmid DNA (pidsBB). Circular DNA is on the left, and linearized plasmid DNA is on the right. “Ladder” is the NEB 2-log DNA ladder. Both circular and linearized DNA is degraded in the samples with ^{Proteus}IdrD-CT. Band running below 1 kB are presumed to be rRNA and tRNA from PURExpress reactions.

FIG. 5A shows the gel of FIG. 2C (^{Rothia}IdrD-CT) with RNA bands visible. “Ladder” is the NEB 2-log DNA ladder. Bands running below 1 kB are presumed to be rRNA and tRNA from PURExpress in vitro translation reactions.

FIG. 5B shows an agarose gel of DNase assays with lambda DNA and IdrD-CTDs from R. aeria with N- and C-terminal truncations. “Ladder” is the NEB 2-log DNA ladder.

FIG. 5C shows an agarose gel of DNase assays with lambda DNA and N- and C-terminal IdrD-CTD truncations of FIG. 2D with RNA bands visible.

FIG. 6A Proteins like IdrD-CT are aligned (using MUSCLE) with predicted secondary structures as colors on top. Alpha-helices (boxed), and beta-sheets (underlined) are identified; intensity of shading reflects confidence of the predictions. The PD-(D/E)XK domain is noted. An arrow marks the IdrD-CT from P. mirabilis strain BB2000.

FIG. 6B shows phylogeny of IdrD-CT based on amino acid alignments. Translated amino acid sequences of the 23 identified IdrD-CT homologs were aligned with muscle, filtered for 70% occupancy, and passed to MrBayes for phylogenetic reconstruction.

FIG. 6C shows a species tree of taxa containing IdrD-CT. 16S rRNA sequences were obtained from published genomes of the taxa represented in FIG. 6B, aligned with muscle, filtered for 70% occupancy, and passed to MrBayes for phylogenetic reconstruction. In both FIGS. 6B and 6C, phyla branches are identified as: dotted: Bacteriodetes; dashed: Proteobacteria; or dot/dash: Actinobacteria). Numbers adjacent to each branch report posterior probability. Scale bars show expected substitutions per site.

### DEFINITIONS

The term “administer,” “administering,” or “administration” refers to implanting, absorbing, ingesting, injecting, inhaling, or otherwise introducing recombinant IdrD protein as described herein, or a composition thereof, in or on a subject.

Bacterial infections include, but are not limited to, gram-negative bacterial infections, gram-positive bacterial infections, and other bacterial infections. Exemplary bacterial infections include, but are not limited to, infections with a gram positive bacteria (e.g., of the phylum Actinobacteria, phylum Firmicutes, or phylum Tenericutes); gram negative bacteria (e.g., of the phylum Aquificae, phylum Deinococcus-Thermus, phylum Fibrobacteres/Chlorobi/Bacteroidetes (FCB), phylum Fusobacteria, phylum Gemmatimonadest, phylum Nitrospirae, phylum Planctomycetes/Verrucomicrobia/Chlamydiae (PVC), phylum Proteobacteria, phylum Spirochaetes, or phylum Synergistetes); or other bacteria (e.g., of the phylum Acidobacteria, phylum Chlroflexi, phylum Chrystiogenetes, phylum Cyanobacteria, phylum Deferrubacteres, phylum Dictyoglomi, phylum Thermodesulfobacteria, or phylum Thermotogae).

The term “boundary formation,” as used herein, refers to a macroscopically visible boundary of up to approximately 3 millimeters (mm) formed when swarming populations of bacteria (e.g., P. mirabilis) meet and recognize each other as non-self. In contrast, swarming populations of bacteria (e.g., P. mirabilis) that meet and recognize each other as self-merge to form a single larger swarm.

The term “coating,” as used herein, refers to a layer of a fragment of IdrD protein covering a surface. The coating can be applied to the surface or impregnated into the material of the surface. The coating may comprise any IdrD protein suitable for inhibiting the growth or motility of bacteria.

An “effective amount” of an IdrD protein described herein refers to an amount sufficient to elicit the desired biological response. An effective amount of a protein described herein may vary depending on such factors as the desired biological endpoint, the pharmacokinetics of the protein, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a protein described herein in a single dose. In certain embodiments, an effective amount is the combined amounts of a protein described herein in multiple doses.

The term “fusion protein,” as may be used herein, refers to a hybrid (e.g., recombinant) polypeptide which comprises protein domains from at least two different proteins. One protein domain may be located at the amino terminal (N terminal) portion of the fusion protein and will contain the free N-terminus (e.g., amino (NH₂) group) of the fusion protein, this protein domain of the fusion protein may be referred to as the “amino-terminal fusion protein” or “amino-terminal fusion protein domain.” Similarly, one protein domain may be located at the carboxy terminal (C terminal) portion of the fusion protein and will contain the free C-terminus (e.g., carboxyl (COOH) group) of the fusion protein, this protein domain of the fusion protein may be referred to as the “carboxy-terminal fusion protein” or “carboxy-terminal fusion protein domain.” A protein may comprise different domains, for example, ^{Proteus}IdrD-CT, ^{Proteus}IdrD-CTD_{39A}, RothiaIdrD-CT, ^{Rothia}IdrD-CTD_{39A}, DR, RD, and/or IdrD protein variant. Any of the fusion proteins provided herein may be produced by any method known in the art. For example, the proteins provided herein may be produced via recombinant protein expression and purification, which is especially suited for fusion proteins comprising a peptide linker. Methods for fusion protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference. In some embodiments, a fusion protein can be encoded by a recombinant nucleic acid (e.g., DNA, RNA).

The term “host cell” as used herein, refers to a cell that can host, replicate, and express a vector as described herein, e.g., a vector comprising a nucleic acid molecule encoding an protein of interest and/or any of the proteins disclosed herein (e.g., ^{Proteus}IdrD-CT, ^{Proteus}IdrD-CT_{D39A}, ^{Rothia}IdrD-CT, ^{Rothia}IdrD-CT_{D39A}, DR, RD, IdrD protein variant, mutant proteins, protein fragments, fusion proteins, etc.).

The term “inhibition,” as used herein, refers to inhibition of a pathogenic bacteria including the inhibition of any desired function or activity of the bacteria, such as bacterial growth, colonization, or swarming. Inhibition of bacterial growth may include inhibition of the size of the bacteria (e.g., pathogenic bacteria) and/or inhibition of the proliferation or multiplication of the bacteria (e.g., pathogenic bacteria). Inhibition of colonization of a bacterium may include inhibition of the amount of bacteria (e.g., a decrease in the amount of bacteria), and may be demonstrated by measuring the amount of the bacteria before and after a treatment. Inhibition or inhibiting includes total and partial reduction of one or more activities of a bacteria (e.g., pathogenic bacteria).

The term “linker,” as may be used herein, refers to a molecule linking two other molecules or moieties. Linkers are well known in the art and can comprise any suitable combination of nucleic acids or amino acids to facilitate the proper function of the structures they join. The linker can be a series of amino acids. The linker can be an amino acid sequence in the case of a linker joining two fusion proteins. For example, a fusion protein, comprising any of the proteins, protein fragments, or protein variants as described herein (e.g., ^{Proteus}IdrD-CT, ^{Proteus}IdrD-CTD_{39A}, ^{Rothia}IdrD-CT, ^{Rothia}IdrD-CTD_{39A}, DR, RD, and/or IdrD protein variant) can be fused to another protein, protein fragment, or protein variant as described herein (e.g., ^{Proteus}IdrD-CT, ^{Proteus}IdrD-CTD_{39A}, RothiaIdrD-CT, RothiaIdrD-CTD_{39A}, DR, RD, and/or IdrD protein variant) and/or another moiety by an amino acid linker sequence. The linker can also be a nucleotide sequence in the case of joining two nucleotide sequences together. In some embodiments, the linker is an organic molecule, functional group, group, polymer, or other chemical moiety. In some embodiments, the linker is a cleavable linker, e.g., the linker comprises a bond that can be cleaved upon exposure to, for example, UV light or a hydrolytic enzyme, such as a protease or esterase. In some embodiments, the linker is 1-100 amino acids in length, for example: 1; 2; 3; 4; 5; 6; 7; 8; 9; 10; 11; 12; 13; 14; 15; 16; 17; 18; 19; 20; 21; 22; 23; 24; 25; 26; 27; 28; 29; 30; 30-35; 35-40; 40-45; 45-50; 50-60; 60-70; 70-80; 80-90; 90-100; 100-150; or 150-200 amino acids in length. In some embodiments, the linker is 5-1,000 nucleotides in length, for example: 5; 6; 7; 8; 9; 10; 11; 12; 13; 14; 15; 16; 17; 18; 19; 20; 21; 22; 23; 24; 25; 26; 27; 28; 29; 30; 30-35; 35-40; 40-45; 45-50; 50-60; 60-70; 70-80; 80-90; 90-100; 100-150; 150-200; 200-300; 300-500; 500-1,000; 1,000-2,000; or 2,000-5,000 nucleotides. In other embodiments, the linker is a chemical bond (e.g., a covalent bond, amide bond, disulfide bond, ester bond, carbon-carbon bond, carbon heteroatom bond). Longer or shorter linkers are also contemplated.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “non-pathogenic bacteria,” as used herein, refers to any known or unknown non-pathogenic bacteria (gram positive or gram negative) and any pathogenic bacteria that has been mutated or converted to a non-pathogenic bacteria. Bacteria may be pathogenic to specific organisms and non-pathogenic to other organisms, and thus bacteria can be utilized in the organism in which it is non-pathogenic.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “pathogenic bacteria,” as used herein, refers to any bacteria or any other organism that is capable of causing or contributing to a disease, disorder, or condition of a host organism.

The “percent identity” of two amino acid sequences is determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

The term “pharmaceutical composition,” as used herein, refers to any compositions prepared by any method known in the art of pharmacology. In general, such preparatory methods include bringing the “active ingredient,” for example, IdrD protein as described herein, into association with a carrier or excipient, and/or one or more other accessory ingredients, and then, if necessary and/or desirable, shaping and/or packaging the product into a desired single- or multi-dose unit.

A “prophylactically effective amount” of a compound described herein is an amount sufficient to prevent a condition, or one or more symptoms associated with the condition or prevent its recurrence. A prophylactically effective amount of an IdrD protein means an amount of an IdrD protein or fragment thereof, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “engineered protein” as used herein refers to a polypeptide which has been modified from its naturally occurring counterpart. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The terms “P. mirabilis IdrD,” “IdrD-CT,” “^{Proteus}IdrD-CT,” and “D,” as used herein, refer interchangeably to IdrD from P. mirabilis. In some embodiments, P. mirabilis IdrD corresponds to IdrD from P. mirabilis strain BB2000 (NCBI Reference Sequence: BB2000_0825; GenBank No. AGS59321.1). In some embodiments, P. mirabilis IdrD corresponds to a fragment of IdrD from P. mirabilis strain BB2000 provided in SEQ ID NO: 1 (amino acid) or SEQ ID NO: 2 (nucleotide)).

(SEQ ID NO: 1)
MCFSARVGAFGEKRVMKYLSGAGYKKVFSVQNNSGHGLDIVALRPDGKFD
IFEVKSSTIGQFSLSSRQATGDDFAKIVLLNDVKKGGYNIIDIDGNVKAI
TSKQARYIYNNIGTTEWVQVNVGRNPKFYDQNITFEEW
(SEQ ID NO: 2)
ATGTGCTTTAGTGCTCGGGTAGGTGCTTTTGGTGAGAAAAGAGTTATGAA
ATACTTATCTGGAGCGGGCTATAAAAAAGTTTTTTCTGTACAAAACAATT
CTGGGCATGGTCTGGATATAGTTGCTTTAAGACCAGATGGAAAATTTGAT
ATTTTTGAAGTTAAAAGTTCGACAATAGGACAATTTTCTTTATCTTCCCG
CCAAGCTACAGGCGATGATTTTGCAAAAATAGTTCTTTTAAACGATGTGA
AAAAAGGAGGTTATAATATTATCGATATAGATGGTAATGTTAAAGCAATT
ACAAGTAAACAAGCTAGATACATTTATAATAACATAGGAACAACCGAGTG
GGTTCAGGTAAATGTTGGCCGAAATCCCAAATTTTATGATCAGAATATAA
CGTTTGAGGAATGGTAG

The terms “mutant P. mirabilis IdrD,” “^{Proteus}IdrD-CT_{D39A},” and “D*,” as used herein, refers interchangeably to mutant IdrD from Proteus mirabilis strain BB2000 having reduced activity as compared to its wild-type counterpart. In some embodiments, mutant IdrD from P. mirabilis is provided by SEQ ID NO: 3 (amino acid) or SEQ ID NO: 4 (nucleotide). The underlined amino acid in SEQ ID NO: 3 corresponds to a single amino acid substitution of aspartic acid (D) with alanine (A) in the predicted active site.

(SEQ ID NO: 3)
MCFSARVGAFGEKRVMKYLSGAGYKKVFSVQNNSGHGLAIVALRPDGKFD
IFEVKSSTIGQFSLSSRQATGDDFAKIVLLNDVKKGGYNIIDIDGNVKAI
TSKQARYIYNNIGTTEWVQVNVGRNPKFYDQNITFEEW
(underlined: mutated residue)
(SEQ ID NO: 4)
ATGTGCTTTAGTGCTCGGGTAGGTGCTTTTGGTGAGAAAAGAGTTATGAA
ATACTTATCTGGAGCGGGCTATAAAAAAGTTTTTTCTGTACAAAACAATT
CTGGGCATGGTCTGGCTATAGTTGCTTTAAGACCAGATGGAAAATTTGAT
ATTTTTGAAGTTAAAAGTTCGACAATAGGACAATTTTCTTTATCTTCCCG
CCAAGCTACAGGCGATGATTTTGCAAAAATAGTTCTTTTAAACGATGTGA
AAAAAGGAGGTTATAATATTATCGATATAGATGGTAATGTTAAAGCAATT
ACAAGTAAACAAGCTAGATACATTTATAATAACATAGGAACAACCGAGTG
GGTTCAGGTAAATGTTGGCCGAAATCCCAAATTTTATGATCAGAATATAA
CGTTTGAGGAATGGTAG
(underlined: codon encoding the mutated residue of
SEQ ID NO: 3)

The terms “Rothia IdrD,” “IdrD-CT,” and “^{Rothia}IdrD-CT,” and “R,” as used herein, refers interchangeably to IdrD from Rothia. In some embodiments, Rothia IdrD corresponds to IdrD from Rothia (NCBI Reference Sequence: WP_023134414.1, SEQ ID NO: 5 (amino acid) or SEQ ID NO: 6 (nucleotide)).

(SEQ ID NO: 5)
MKYLEGTGRYKKVSSIQNASGNGLDIVALRLDGKYDIFEVKSSKRGNFRL
SERQQKGGKCFAEQVLMKDVKKGGYFMKGLDGKETPIGPKEAQEIFNNID
KTETVFVDMNSKFRATRITFGLW
(SEQ ID NO: 6)
ATGACCTACGTGCAGCGCTTGGGTACTGCCGGCGAGCGCAGGGTAATGAA
ATATCTGGAAGGTACGGGATACAAAAAAGTCTTCTCTATTCAAAATGCCT
CCGGAAACGGCTTGGATATTGTCGCATTGAGACCGGATGGGAAATATGAC
ATATTTGAAGTTAAAAGTTCTAAACGTGGAAAATTTAAACTAAGCGAAAG
ACAGCAGAAAGGCGGTAAATGTTTTGCTGAGCAAGTGTTGACGGAAGATG
TAACGGATAAGAAAAAAGGCGGATATTTTATGAAAGGCTTAGACGGCAAA
AAGACGCCTCTTAATAAGAAGAAGGCGCAAGAGATATTTAATAATATAGA
TAAAACCGAAACTGTCTTTGTTGATATGAATCATAAATTTCAAGCAACCC
GCATGACATTTAGCCCATGGTAA

The terms “mutant Rothia IdrD,” “, ^{Rothia}IdrD-CT_{D39A},” and “R*,” as used herein, refers interchangeably to mutant IdrD from Rothia strain having reduced activity as compared to its wild-type counterpart. In some embodiments, mutant IdrD from Rothia is provided by SEQ ID NO: 7 (amino acid) or SEQ ID NO: 8 (nucleotide). The underlined amino acid in SEQ ID NO: 7 corresponds to a single amino acid substitution of aspartic acid (D) with alanine (A) in the predicted active site.

(SEQ ID NO: 7)
MKYLEGTGRYKKVSSIQNASGNGLAIVALRLDGKYDIFEVKSSKRGNFRL
SERQQKGGKCFAEQVLMKDVKKGGYFMKGLDGKETPIGPKEAQEIFNNID
KTETVFVDMNSKFRATRITFGLW
(underline: mutated residue)
(SEQ ID NO: 8)
ATGACCTACGTGCAGCGCTTGGGTACTGCCGGCGAGCGCAGGGTAATGAA
ATATCTGGAAGGTACGGGATACAAAAAAGTCTTCTCTATTCAAAATGCCT
CCGGAAACGGCTTGGCTATTGTCGCATTGAGACCGGATGGGAAATATGAC
ATATTTGAAGTTAAAAGTTCTAAACGTGGAAAATTTAAACTAAGCGAAAG
ACAGCAGAAAGGCGGTAAATGTTTTGCTGAGCAAGTGTTGACGGAAGATG
TAACGGATAAGAAAAAAGGCGGATATTTTATGAAAGGCTTAGACGGCAAA
AAGACGCCTCTTAATAAGAAGAAGGCGCAAGAGATATTTAATAATATAGA
TAAAACCGAAACTGTCTTTGTTGATATGAATCATAAATTTCAAGCAACCC
GCATGACATTTAGCCCATGGTAA
(underlined: codon encoding the mutated residue of
SEQ ID NO: 7)

The terms “fusion IdrD protein” refers to an IdrD protein comprising a fragment of P. mirabilis and a fragment of Rothia. In some embodiments, “DR,” as used herein, refers to a fragment of IdrD from P. mirabilis fused to a fragment of IdrD from Rothia as provided by SEQ ID NO: 9 (amino acid) or SEQ ID NO: 10 (nucleotide).

(SEQ ID NO: 9)
MCFSARVGAFGEKRVMKYLSGAGYKKVFSVQNNSGHGLDIVALRPDGKFD
IFEVKSSTIGQFSLSSRQATGDDFAKIVLLNDVKKKKGGYFMKGLDGKKT
PLNKKKAQEIFNNIDKTETVFVDMNHKFQATRMTFSPW
(underlined: fragment of IdrD from P. mirabilis)
(SEQ ID NO: 10)
ATGTGCTTTAGTGCTCGGGTAGGTGCTTTTGGTGAGAAAAGAGTTATGAA
ATACTTATCTGGAGCGGGCTATAAAAAAGTTTTTTCTGTACAAAACAATT
CTGGGCATGGTCTGGATATAGTTGCTTTAAGACCAGATGGAAAATTTGAT
ATTTTTGAAGTTAAAAGTTCGACAATAGGACAATTTTCTTTATCTTCCCG
CCAAGCTACAGGCGATGATTTTGCAAAAATAGTTCTTTTAAACGATGTGA
AAAAAGAAAAAGGCGGATATTTTATGAAAGGCTTAGACGGCAAAAAGACG
CCTCTTAATAAGAAGAAGGCGCAAGAGATATTTAATAATATAGATAAAAC
CGAAACTGTCTTTGTTGATATGAATCATAAATTTCAAGCAACCCGCATGA
CATTTAGCCCATGGTAA
(underlined: fragment of IdrD from P. mirabilis)

In some embodiments, “RD,” as used herein, refers to a fragment of IdrD from Rothia fused to a fragment of IdrD from P. mirabilis as provided by SEQ ID NO: 11 (amino acid) or SEQ ID NO: 12 (nucleotide).

(SEQ ID NO: 11)
MTYVQRLGTAGERRVMKYLEGTGYKKVFSIQNASGNGLDIVALRPDGKYD
IFEVKSSKRGKFKLSERQQKGGKCFAEQVLTEDVTDKGGYNIIDIDGNVK
AITSKQARYIYNNIGTTEWVQVNVGRNPKFYDQNITFEEW
(underlined: fragment of IdrD from P. mirabilis)
(SEQ ID NO: 12)
ATGACCTACGTGCAGCGCTTGGGTACTGCCGGCGAGCGCAGGGTAATGAA
ATATCTGGAAGGTACGGGATACAAAAAAGTCTTCTCTATTCAAAATGCCT
CCGGAAACGGCTTGGATATTGTCGCATTGAGACCGGATGGGAAATATGAC
ATATTTGAAGTTAAAAGTTCTAAACGTGGAAAATTTAAACTAAGCGAAAG
ACAGCAGAAAGGCGGTAAATGTTTTGCTGAGCAAGTGTTGACGGAAGATG
TAACGGATAAAGGAGGTTATAATATTATCGATATAGATGGTAATGTTAAA
GCAATTACAAGTAAACAAGCTAGATACATTTATAATAACATAGGAACAAC
CGAGTGGGTTCAGGTAAATGTTGGCCGAAATCCCAAATTTTATGATCAGA
ATATAACGTTTGAGGAATGGTAA
(underlined: fragment of IdrD from P. mirabilis)

In some embodiments, “IdrD protein variant,” as used herein, refers to a full-length IdrD protein comprising one or more modifications. Examples of modifications include, but are not limited to, a mutation, an insertion, and a deletion. For example, an IdrD protein variant comprises a full-length IdrD protein sequence from P. mirabilis (NCBI GenBank Accession Number: AGS59321.1, SEQ ID NO: 13 (amino acid) or SEQ ID NO: 14 (nucleotide)) comprising one or more mutations.

(SEQ ID NO: 13)
MGNEFFAAKQGDKLMHSSIFADIVCGVVKGAVYAAIGTAAVALTIGTGGMATAAGVAIFAGA
GLAAGFTIGGLIDDAADWVADGIDSLFGLSNPDGEIDTGSKNVRVKGKGAARAAGKLPSPVL
LASIAADNTPPKEEKGTEIALRVLKNTAMMATGAFSLLAGKVLGSQTNNAAPIALDPSEFPE
YPSPEQGFFDSILSPVKAAKHPNAEPMPLDTITCDKWHCASAPYFLAEGSKKVQINSQPACR
NGDRSTCEAKISDTQEKGIVRIGGGSVVVRDIMSGRNPFAEMFGEILGGLAVGAASTLFRRG
GLKAIKQCLNSVGCTLVSEIASTAVGSMVVASVTQAFNSVRHPVHAATGAKVLNGEDDTDFI
IDGVYPLVWSRIYQSRNTGENRLGRGWSMPFDVFLTIDDTGKGLENENIYYHDMSGRRLALG
KIALGQKVFYQDEGFTVYRTTNNLFLVESAEGDYQLFEANPHKINTLRLMKSADRHNNALHY
RYANDGELVQIHDDAYLTDIRLHYDEITQRLQSVTRHQGQEEKTLVTYTYDAQQRLVQVTNA
DKRVTRRFGWDDESGLMAMHQYATGVSSHYRWQRFDAFTIEDNEPEWRVVEHWLKDGKRCLE
HTELTYDLAQRTLTTVETGGETTFRRWNEQQQIIEYTNALNETWWFEWDTSRLLTKAIAPDG
SEWGYTYDERGNLTQSTDPEQQSTCYDWDKDFAFPTAQTLPNGAAWHWEYNEHGDIRRVIDP
LGHITRLAWDDQGLCLGQVDAKGNETHYRYNARGQLIEQRDCSGYPTTLTYDDWGQLRSLTN
AQNETTTYTFSEAGLLLTERLPDGTENRYDYDATGQLVGITDAGERHILLRRNRRGQVIARR
DPAGHWLHFHYDTFGRMQALENEQGEQYRFEYDALHRLTDEHDLIGQQKHYQYDVMGNVTQI
KTTPGPCVDTPMPLSPLVTTFGYDKVGRLLFRENADYRTEYLYQPLSVTLRRVPMAIWHEAE
RTGTTARVEYQDALTFTYDKVGQLVREASARDDYQHHYDVLGNITRTELPHQRAFEYLYYGS
GHLQQTQWRDNAQLTVLAEYQRDRLHRETLRTSGALDNETGYDCRGRITHQVARQMNASQFV
TPVIDRRYRWDKRNQLIERSVSYGQTGEVFTAGHWYYHSYQYDPLGQLTAHLGSVQTEHFLY
DAAANLLTRPHSEAPHNQVQGSDKYDYRYDGFGRMVSRYEKGSSSGQRYHYDSDHRIIAVDI
DQGPLGYQRAEYRYDILGRRIEKRLWKASAIANTVTYHQHEPDEVYTFGWVGMRLVSEHSSA
APHTTVYHAYNDQSYTPLARIECTDNPLNPQRAIYYTHSSLSGLPEALTNSEGEIVWQGQYS
VWGHLQRQTRPTSTFNREQNLRFQGQYFDKETGLHYNTFRYYAPDLGRFTQQDPIGLAGGIN
LYAYAPNPLTWVDPWGWSCFSARVGAFGEKRVMKYLSGAGYKKVFSVQNNSGHGLDIVALRP
DGKFDIFEVKSSTIGQFSLSSRQATGDDFAKIVLLNDVKKGGYNIIDIDGNVKAITSKQARY
IYNNIGTTEWVQVNVGRNPKFYDQNITFEEW
(SEQ ID NO: 14)
ATGGGAAATGAATTTTTCGCAGCCAAGCAAGGCGATAAATTAATGCACTCGTCAATCTTCGC
CGATATCGTGTGTGGTGTGGTTAAAGGGGCGGTTTATGCCGCGATAGGGACGGCCGCTGTGG
CGTTGACTATTGGTACAGGTGGTATGGCAACGGCCGCCGGTGTTGCCATTTTTGCCGGTGCA
GGGCTGGCGGCGGGCTTTACCATCGGTGGATTGATTGATGATGCCGCTGATTGGGTCGCGGA
TGGGATCGATAGCTTGTTCGGGCTAAGTAACCCTGATGGTGAAATCGATACTGGCTCGAAAA
ATGTACGTGTCAAAGGCAAGGGAGCCGCTCGCGCAGCGGGAAAATTACCGTCACCCGTACTA
TTAGCCTCAATTGCGGCAGATAATACCCCACCGAAAGAAGAAAAAGGCACCGAGATTGCCTT
ACGCGTATTGAAAAATACCGCCATGATGGCGACTGGGGCATTTTCTTTATTGGCTGGTAAGG
TGCTAGGGAGCCAAACCAACAATGCGGCACCCATAGCGCTCGATCCCTCCGAGTTTCCCGAA
TACCCCTCACCGGAGCAAGGTTTTTTTGACAGTATTCTTTCCCCAGTAAAAGCTGCGAAACA
CCCGAATGCTGAACCTATGCCTTTGGATACCATCACGTGCGATAAATGGCATTGTGCTAGCG
CCCCTTACTTTTTAGCCGAAGGGTCGAAAAAAGTCCAAATTAATAGCCAACCGGCGTGTCGT
AACGGTGATAGAAGCACTTGTGAAGCCAAAATCAGTGATACGCAAGAAAAAGGGATTGTGCG
TATTGGCGGTGGCAGTGTGGTGGTACGCGATATTATGAGTGGGCGTAACCCCTTTGCCGAAA
TGTTTGGTGAAATTCTCGGTGGCCTTGCTGTTGGAGCGGCGTCTACCCTGTTTCGCAGAGGG
GGACTCAAGGCGATTAAACAGTGTTTGAATAGTGTAGGCTGTACTTTGGTGAGTGAAATCGC
CAGTACCGCGGTGGGCAGTATGGTAGTCGCCTCGGTGACCCAAGCCTTTAATAGTGTGCGTC
ACCCCGTGCATGCGGCTACGGGGGCGAAAGTCCTAAATGGAGAGGATGATACCGATTTTATT
ATTGATGGTGTGTACCCGTTAGTCTGGTCGCGTATTTACCAAAGTCGTAATACGGGTGAAAA
TCGTTTAGGACGCGGTTGGTCGATGCCGTTTGATGTCTTTTTGACAATTGACGACACAGGTA
AAGGGCTTGAGAATGAAAACATTTATTATCATGACATGTCGGGGAGGCGTTTAGCGTTAGGA
AAAATCGCCCTTGGACAGAAAGTGTTCTATCAAGATGAAGGCTTTACCGTCTATCGCACGAC
AAACAATCTTTTCCTTGTTGAATCGGCGGAAGGTGATTATCAACTTTTCGAAGCCAATCCAC
ATAAAATCAATACACTGCGCTTAATGAAAAGTGCTGATCGTCATAATAACGCTTTACACTAC
CGCTATGCTAATGACGGTGAATTGGTACAAATTCATGATGATGCGTATCTGACCGATATCCG
GTTACATTATGATGAAATCACCCAACGCTTACAGTCGGTGACCCGCCACCAAGGACAAGAAG
AAAAAACGCTGGTCACTTATACTTATGATGCACAACAGCGTTTAGTGCAAGTCACTAATGCG
GATAAGCGAGTGACTCGTCGTTTTGGCTGGGATGATGAATCGGGTTTAATGGCCATGCACCA
ATATGCCACAGGGGTCAGTAGTCATTATCGTTGGCAACGTTTTGATGCCTTTACCATTGAAG
ACAATGAACCTGAGTGGCGAGTGGTTGAACACTGGCTTAAAGACGGTAAACGCTGTTTAGAG
CATACCGAACTGACGTATGATTTAGCTCAACGAACCCTCACCACCGTGGAAACGGGGGGTGA
AACCACCTTCCGTCGCTGGAATGAACAACAGCAAATTATCGAATACACCAATGCATTGAATG
AAACGTGGTGGTTTGAATGGGATACCAGCCGCTTATTAACGAAAGCAATAGCCCCAGATGGC
AGTGAATGGGGGTATACCTATGATGAGCGCGGTAATTTAACCCAATCGACCGATCCGGAACA
ACAATCGACTTGCTATGATTGGGACAAAGATTTTGCGTTTCCCACGGCACAAACTTTGCCAA
ATGGGGCGGCTTGGCATTGGGAGTACAATGAGCACGGCGATATTCGTCGTGTGATTGACCCG
CTTGGCCATATCACGCGCTTGGCGTGGGATGACCAAGGCCTGTGTCTGGGGCAAGTGGATGC
TAAGGGTAATGAAACCCACTATCGCTATAATGCCCGCGGTCAGTTAATCGAGCAACGGGACT
GTTCGGGTTATCCCACCACCTTGACCTACGATGATTGGGGACAACTTCGCTCGTTGACCAAT
GCACAGAATGAAACCACGACTTACACCTTTAGTGAAGCGGGGTTGTTGTTAACAGAGCGCTT
ACCGGATGGGACAGAAAACCGTTATGACTATGATGCCACCGGGCAATTAGTGGGAATAACGG
ATGCCGGAGAGCGCCATATTCTGTTGCGCCGTAACCGTCGTGGACAAGTGATAGCCCGACGC
GATCCGGCAGGGCATTGGTTGCATTTTCACTATGATACTTTCGGGCGCATGCAGGCACTGGA
GAATGAACAGGGCGAGCAGTACCGCTTTGAGTATGATGCCTTGCATCGTTTAACCGATGAAC
ATGATCTTATCGGACAACAAAAGCACTATCAGTACGATGTGATGGGGAATGTCACCCAGATA
AAAACTACCCCGGGGCCTTGCGTTGATACGCCGATGCCCTTATCCCCGCTGGTGACCACCTT
CGGTTACGATAAAGTGGGACGGTTGCTGTTTCGAGAAAACGCCGATTATCGCACGGAATACC
TTTATCAACCTTTGAGTGTGACGTTACGCCGAGTCCCCATGGCGATATGGCATGAAGCCGAA
CGCACCGGCACCACCGCCCGCGTAGAGTATCAAGACGCCCTGACCTTTACCTACGATAAAGT
GGGGCAGTTGGTTCGAGAAGCCAGTGCCCGCGATGATTATCAGCATCACTATGATGTGCTGG
GTAATATCACTCGCACCGAGTTGCCCCATCAAAGGGCATTTGAATACCTGTATTACGGCTCT
GGGCATTTACAACAAACGCAATGGCGAGATAATGCGCAACTGACGGTATTGGCGGAATATCA
ACGAGACCGTTTACACCGAGAAACGTTGCGCACCAGTGGCGCCTTAGACAATGAAACGGGCT
ATGACTGTCGGGGACGTATTACGCACCAAGTGGCGCGCCAAATGAATGCCTCACAGTTTGTG
ACCCCGGTGATTGACCGTCGTTATCGTTGGGATAAACGCAATCAACTTATCGAGCGCAGTGT
CAGTTATGGTCAAACAGGCGAGGTTTTTACCGCCGGACATTGGTACTACCACAGTTATCAGT
ACGACCCGCTGGGGCAATTGACGGCGCATTTAGGGTCGGTGCAAACCGAACACTTTCTGTAT
GATGCGGCGGCCAATTTACTGACTCGTCCGCACAGCGAAGCACCGCATAACCAAGTACAAGG
CAGTGATAAGTATGATTATCGCTACGACGGTTTTGGTCGCATGGTCTCCCGCTATGAAAAAG
GCAGTAGCTCGGGACAACGTTATCACTATGACAGTGACCACCGCATTATCGCGGTGGATATC
GACCAAGGGCCCTTAGGGTATCAGCGAGCGGAATACCGCTACGATATTTTAGGGCGCCGGAT
TGAAAAACGGTTATGGAAAGCCAGTGCGATTGCCAACACAGTCACCTATCACCAACATGAGC
CGGATGAAGTGTACACTTTTGGCTGGGTAGGGATGCGACTGGTGTCTGAGCACAGCAGCGCA
GCGCCCCATACCACGGTTTATCATGCATACAATGACCAAAGTTATACGCCTCTTGCTCGCAT
TGAATGCACGGATAACCCGCTTAATCCGCAACGGGCGATTTATTATACGCACAGCAGTTTAA
GTGGCTTACCGGAAGCGTTGACCAACAGTGAGGGTGAAATAGTTTGGCAAGGGCAATACAGT
GTTTGGGGACATTTACAGCGTCAAACCCGCCCCACAAGCACATTTAATCGTGAACAGAACCT
GCGCTTTCAAGGGCAGTATTTCGACAAAGAGACGGGGTTACATTATAATACGTTCAGGTACT
ATGCCCCGGATTTAGGCCGTTTTACCCAGCAAGACCCAATAGGGTTGGCAGGGGGCATAAAC
TTATATGCTTATGCGCCAAATCCATTGACATGGGTGGATCCGTGGGGTTGGAGTTGCTTTAG
TGCTCGGGTAGGTGCTTTTGGTGAGAAAAGAGTTATGAAATACTTATCTGGAGCGGGCTATA
AAAAAGTTTTTTCTGTACAAAACAATTCTGGGCATGGTCTGGATATAGTTGCTTTAAGACCA
GATGGAAAATTTGATATTTTTGAAGTTAAAAGTTCGACAATAGGACAATTTTCTTTATCTTC
CCGCCAAGCTACAGGCGATGATTTTGCAAAAATAGTTCTTTTAAACGATGTGAAAAAAGGAG
GTTATAATATTATCGATATAGATGGTAATGTTAAAGCAATTACAAGTAAACAAGCTAGATAC
ATTTATAATAACATAGGAACAACCGAGTGGGTTCAGGTAAATGTTGGCCGAAATCCCAAATT
TTATGATCAGAATATAACGTTTGAGGAATGGTAG

The nucleic acids, proteins, mutant proteins, protein fragments, fusion proteins, and/or protein variants (e.g., D, D*, R, R*, DR, RD, mutant IdrD, IdrD protein fragment, and/or IdrD protein variant) as described herein, may further comprise a FLAG epitope (e.g., tag). In some embodiments, the FLAG epitope (e.g., tag) comprises SEQ ID NO: 15.

(SEQ ID NO: 15)
DYKDDDDK

In some embodiments, the nucleic acids comprise a sequence encoding a FLAG epitope (e.g., tag). In some embodiments, the nucleic acid sequence encoding the FLAG epitope (e.g., tag) comprises SEQ ID NO: 16.

(SEQ ID NO: 16)
GACTACAAGGACGACGATGACAAG

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence. In some embodiments, a recombinant protein or nucleic acid comprises a variant of a naturally occurring protein or nucleic acid and/or a truncation of a naturally occurring protein or nucleic acid. In some embodiments, a recombinant protein comprises a fusion protein and/or a tagged protein.

The term “subject,” as used herein, refers to any animal subject including humans, laboratory animals (e.g., primates, rats, mice), livestock (e.g., cows, sheep, goats, pigs, turkeys, chickens), and household pets (e.g., dogs, cats, rodents). The subject may be healthy, or may be suffering from a bacterial infection, or may be at risk of developing or transmitting to others a bacterial infection.

The term “swarming,” as used herein, refers to translocation of a bacterial population across solid or semi-solid surfaces. In some embodiments, swarming is rapid (approximately 2-10 micrometers per second (km/s) and coordinated. Flagellated bacteria are capable of both moving in association with other cells in a thin film of liquid over a moist surface (e.g., swarming) or moving independently in bulk liquid (e.g., swimming).

A “therapeutically effective amount” of a protein described herein is an amount sufficient to provide a therapeutic benefit in the treatment of a condition or to delay or minimize one or more symptoms associated with the condition. A therapeutically effective amount of a protein means an amount of IdrD protein, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The terms “treatment,” “treat,” and “treating” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “vector,” as used herein, refers to any nucleic acids capable of transferring genetic material into a cell (e.g., bacteria). The vector may be linear or circular in topology and includes, but is not limited to, plasmids or bacteriophages. The vector may include amplification genes, enhancers, or selection markers and may or may not be integrated into the genome of the host organism. The term “expression vector”, as used herein, refers to a nucleic acid construct comprising nucleic acid elements sufficient for the expression of a protein of interest (e.g., D, D*, R, R*, DR, RD, IdrD protein variant, mutant IdrD, IdrD fusion protein, etc). Typically, an expression vector comprises a nucleic acid encoding a protein of interest operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In some embodiments, the promoter is a heterologous promoter. The term “heterologous promoter,” as used herein, refers to a promoter that is not found to be operatively linked to a given encoding sequence in nature (e.g., in its native environment). In some embodiments, an expression vector may comprise additional elements, for example, an intron, an enhancer, a polyadenylation site, and/or other elements known to affect expression levels of the encoding sequence. Without wishing to be bound by theory, inclusion of an intron in an expression vector, for example, between the transcriptional start site and an encoding nucleic acid sequence, for example, a protein-encoding cDNA sequence, is believed to result in increased expression levels of the encoding nucleic acid and the encoded protein of interest as compared to an expression vector not including an intron.

### DETAILED DESCRIPTION OF CERTAIN EMBODIMENTS OF THE INVENTION

The present disclosure provides proteins, nucleic acids, vectors, cells, methods, kits, and compositions for inhibiting bacterial growth on a surface, treating a disease (e.g., a disease associated with bacteria trapping neutrophil extracellular traps (NETs)), treating or preventing a bacterial infection, and cleaving DNA using an IdrD protein, for example, an IdrD protein variant or a fragment of a full-length IdrD protein.

IdrD Protein

Some aspects of this disclosure provide an IdrD protein. In some embodiments, the IdrD protein is an IdrD variant comprising an amino acid sequence that is not identical to the amino acid sequence of a naturally occurring IdrD protein (e.g., a Proteus mirabilis IdrD protein). In some embodiments, the IdrD protein variant comprises a full-length IdrD protein comprising one or more modifications, e.g., a mutation (e.g., substitution), an insertion, and/or a deletion. In some embodiments, the IdrD protein, comprises an IdrD protein, IdrD protein fragment, mutant IdrD protein, IdrD protein variant, and/or fusion protein.

In some embodiments, the IdrD protein variant comprises a full-length IdrD protein from P. mirabilis comprising one or more mutations. In some embodiments, the IdrD protein variant comprises a full-length IdrD protein from P. mirabilis comprising one or more insertions. In some embodiments, the IdrD protein variant comprises a full-length IdrD protein from P. mirabilis comprising one or more deletions. In some embodiments, the IdrD protein variant comprises a full-length protein from P. mirabilis comprising one or more mutations and one or more insertions. In some embodiments, the IdrD protein variant comprises a full-length protein from P. mirabilis comprising one or more mutations and one or more deletions. In some embodiments, the IdrD protein variant comprises a full-length protein from P. mirabilis comprising one or more insertions and one or more deletions. In some embodiments, the IdrD protein variant comprises a full-length protein from P. mirabilis comprising one or more mutations, one or more insertions, and one or more deletions.

In some embodiments, the IdrD protein is a fragment of a full-length IdrD protein. In some embodiments, the fragment of IdrD protein comprises an amino acid sequence that is identical to the amino acid sequence of a naturally occurring IdrD protein, e.g., a Proteus mirabilis IdrD protein. In some embodiments, the fragment of IdrD protein comprises an amino acid sequence that is not identical to the amino acid sequence of a naturally occurring IdrD protein, e.g., a Proteus mirabilis IdrD protein.

In some embodiments, the fragment of IdrD protein comprises one or more modifications, e.g., one or more mutations, one or more insertions, and/or one or more modifications. In some embodiments, the fragment of IdrD protein comprises one or more mutations (e.g., a fragment of IdrD protein from P. mirabilis comprising one or more mutations).

In some embodiments, the one or more mutations in any of the proteins, protein variants, protein fragments, mutant proteins, or fusion proteins as disclosed herein, is at least one, at least two, at least three, at least four, at least five, at least six, at least seven, at least eight, at least nine, at least ten, at least fifteen, at least twenty, or at least twenty-five mutations. In some embodiments, the one or more insertions is at least one, at least two, at least three, at least four, at least five, at least six, at least seven, at least eight, at least nine, at least ten, at least fifteen, at least twenty, or at least twenty-five insertions. In some embodiments, the one or more deletions is at least one, at least two, at least three, at least four, at least five, at least six, at least seven, at least eight, at least nine, at least ten, at least fifteen, at least twenty, or at least twenty-five deletions.

In some embodiments, the IdrD protein variant comprises an amino acid sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identical to the amino acid sequence provided by SEQ ID NO: 13, which corresponds to an amino acid sequence of IdrD protein from P. mirabilis.

In some embodiments, the fragment of IdrD protein from P. mirabilis comprises an amino acid sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identical to the amino acid sequence provided by SEQ ID NO: 1.

In some embodiments, the fragment of IdrD protein from P. mirabilis comprises one or more mutations. In some embodiments, the one or more mutations is an insertion into the amino acid sequence provided by SEQ ID NO: 1. In some embodiments, the one or more mutations is a deletion into the amino acid sequence provided by SEQ ID NO: 1. In some embodiments, the one or more mutations is a mutation to amino acid residue 39 of the amino acid sequence provided by SEQ ID NO: 1. In some embodiments, the mutation at amino acid residue 39 is a substitution of aspartic acid with alanine. In some embodiments, the fragment of IdrD protein from P. mirabilis comprising a mutation at amino acid residue 39 as provided in SEQ ID NO: 3.

Some aspects of this disclosure provide IdrD protein comprising a fragment of IdrD protein from Rothia. In some embodiments, the fragment of IdrD protein from Rothia comprises an amino acid sequence that is at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identical to the amino acid sequence provided by SEQ ID NO: 5.

In some embodiments, the fragment of IdrD protein from Rothia comprises one or more mutations. In some embodiments, the one or more mutations is an insertion into the amino acid sequence provided by SEQ ID NO: 5. In some embodiments, the one or more mutations is a deletion into the amino acid sequence provided by SEQ ID NO: 5. In some embodiments, the one or more mutations is a mutation to amino acid residue 25 of the amino acid sequence provided by SEQ ID NO: 5. In some embodiments, the mutation at amino acid residue 25 is a substitution of aspartic acid with alanine. In some embodiments, the fragment of IdrD protein from Rothia comprising a mutation at amino acid residue 25 as provided in SEQ ID NO: 7.

In some embodiments, the IdrD protein is a recombinant IdrD protein. In some embodiments, the IdrD protein is an IdrD fusion protein. In some embodiments, the IdrD fusion protein is formed, for example, by an in-frame gene fusion resulting in the expression of an IdrD protein or fragment thereof fused to a second protein, such as an affinity tag for purification or identification, a fluorescent protein for in situ visualization of the fusion protein, or a protein that promotes bacterial uptake of the fusion protein. In some embodiments, the fragment of an IdrD protein comprises a sequence with at least one amino acid less than a full length IdrD protein from which it is generated (e.g., P. mirabilis, Rothia). In some embodiments, the at least one amino acid is deleted from an end of the protein (e.g., N- or C-terminus truncation). In some embodiments, the at least one amino acid is deleted from an internal portion of the amino acid sequence. In some embodiments, where there is more than one amino acid deleted, a substitution may be made. In some embodiments, where more than one amino acid is deleted the deletions may be contiguous. In some embodiments, where more than one amino acid is deleted the deletions may be non-contiguous. Fragments of the fusion protein sequences disclosed herein are identified as originating from P. mirabilis and/or Rothia. In some embodiments, the identified fragments may be severed from the other fragment and used in other fusion proteins. In some embodiments, the fragments have different or altered IdrD activity (e.g., increase, decreased, aberrant) as compared to full-length IdrD as described herein. In some embodiments, the IdrD fusion protein comprises a fragment of IdrD protein from a first bacteria (e.g., P. mirabilis) and a fragment of IdrD protein from a second bacteria (e.g., Rothia). In some embodiments, the IdrD fusion protein comprises a fragment of IdrD protein from P. mirabilis fused to a fragment of IdrD protein from Rothia. In some embodiments, the IdrD fusion protein comprises a fragment of IdrD protein from P. mirabilis fused to a fragment of IdrD protein from Rothia provided having a sequence with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to SEQ ID NO: 9. In some embodiments, the IdrD fusion protein comprises a fragment of IdrD protein from P. mirabilis fused to a fragment of IdrD protein from Rothia provided in SEQ ID NO: 9. In some embodiments, the IdrD fusion protein comprises a fragment of IdrD protein from Rothia fused to a fragment of IdrD protein from P. mirabilis. In some embodiments, the IdrD fusion protein comprises a fragment of IdrD protein from P. mirabilis fused to a fragment of IdrD protein from Rothia provided having a sequence with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to SEQ ID NO: 11. In some embodiments, the IdrD fusion protein comprises a fragment of IdrD protein from Rothia fused to a fragment of IdrD protein from P. mirabilis provided in SEQ ID NO: 11. In some embodiments, the IdrD fusion protein comprises a fragment of IdrD protein from P. mirabilis fused to a fragment of IdrD protein from Rothia at the N-terminus of the IdrD protein from Rothia. In some embodiments, the IdrD fusion protein comprises a fragment of IdrD protein from P. mirabilis fused to a fragment of IdrD protein from Rothia at the C-terminus of the IdrD protein from Rothia. In some embodiments, the IdrD fusion protein comprises a fragment of IdrD protein from Rothia fused to a fragment of IdrD protein from P. mirabilis at the N-terminus of the IdrD protein from P. mirabilis. In some embodiments, the IdrD fusion protein comprises a fragment of IdrD protein from Rothia fused to a fragment of IdrD protein from P. mirabilis at the C-terminus end of the IdrD protein from P. mirabilis.

In some embodiments, the fusion protein comprises a linker between the multiple components (e.g., fragments, tags, proteins, protein variants) of the fusion proteins described herein. In some embodiments, the IdrD fusion protein comprises a fragment of IdrD protein from Rothia fused to a fragment of IdrD protein from P. mirabilis by means of a linker.

In some embodiments, the IdrD protein comprises one or more distinct amino acid sequences. For example, the IdrD protein may comprise a mixture of a fragment of IdrD protein from P. mirabilis and a fragment of IdrD from Rothia. In some embodiments, the IdrD protein may comprise a mixture of a fragment of IdrD and an IdrD fusion protein. For example, the IdrD protein may comprise a mixture of a fragment of IdrD protein from P. mirabilis and an IdrD fusion protein comprising a fragment of IdrD protein from P. mirabilis fused to a fragment of IdrD protein from Rothia.

Compositions

Aspects of the present disclosure provide compositions comprising an IdrD protein as described herein. In some embodiments, the present disclosure provides compositions comprising a nucleic acid encoding an IdrD protein as described herein. In some embodiments, the composition comprises a nucleic acid encoding fragment of IdrD protein from P. mirabilis. In some embodiments, the composition comprises a nucleic acid encoding fragment of IdrD protein from P. mirabilis, wherein the protein comprises a sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to SEQ ID NO: 1 or 3. In some embodiments, the composition comprises a nucleic acid encoding fragment of IdrD protein from P. mirabilis, wherein the protein comprises a sequence as set forth in SEQ ID NO: 1 or 3. In some embodiments, the composition comprises a nucleic acid encoding a fragment of IdrD protein from P. mirabilis provided by SEQ ID NO: 2 or SEQ ID NO: 4. In some embodiments, the composition comprises a nucleic acid encoding a fragment of IdrD protein from P. mirabilis wherein the nucleic acid comprises a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to SEQ ID NO: 2 or 4. In some embodiments, the composition comprises a nucleic acid encoding fragment of IdrD protein from Rothia. In some embodiments, the composition comprises a nucleic acid encoding fragment of IdrD protein from Rothia, wherein the protein comprises a sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to SEQ ID NO: 5 or 7. In some embodiments, the composition comprises a nucleic acid encoding fragment of IdrD protein from Rothia, wherein the protein comprises a sequence as set forth in SEQ ID NO: 5 or 7. In some embodiments, the composition comprises a nucleic acid encoding a fragment of IdrD protein from Rothia provided by SEQ ID NO: 6 or SEQ ID NO: 8. In some embodiments, the composition comprises a nucleic acid encoding a fragment of IdrD protein from Rothia wherein the nucleic acid comprises a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to SEQ ID NO: 6 or 8. In some embodiments, the composition comprises a nucleic acid encoding an IdrD fusion protein provided by SEQ ID NO: 10 or SEQ ID NO: 12. In some embodiments, the composition comprises a nucleic acid encoding an IdrD fusion protein having a nucleotide sequence having at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to SEQ ID NO: 10 or 12.

Aspects of the present disclosure provide cells for producing an IdrD protein as described herein. In some embodiments, the cells are genetically engineered to express the IdrD protein. In some embodiments, the cells comprise a vector encoding the IdrD protein, for example, an expression vector. Vectors are introduced into cells by standard methods including electroporation or infection by viral vectors.

Any type of cell may be used according to methods and compositions described herein. In some embodiments, the cell is a prokaryotic cell. In some embodiments, the cell is an eukaryotic cell. In some embodiments, the cell is a bacterial cell. In some embodiments, the cell is a yeast cell. In some embodiments, the cell is a mammalian cell. In some embodiments, the cell is a human cell.

Any of the IdrD proteins or fragments thereof described herein may be provided in a kit. For example, the kit comprises a full-length IdrD protein comprising an amino acid sequence that is not identical to the amino acid sequence of a naturally occurring IdrD protein, e.g., a Proteus mirabilis IdrD protein. In some embodiments, the kit comprises a fragment of a full-length IdrD protein. In some embodiments, the kit comprises a fusion protein comprising a fragment of IdrD protein from a first bacteria (e.g., P. mirabilis) and a fragment of IdrD protein from a second bacteria (e.g., Rothia). In some embodiments, the kit comprises a nucleic acid for expressing a IdrD protein or fragment thereof described herein.

In some embodiments, the kit further comprises at least one reagent for performing a method described herein including, but not limited to, methods for inhibiting bacterial biofilm formation on a surface, methods for treating a disease associated with bacteria trapping neutrophil extracellular traps (NETs), methods of treating or preventing a bacterial infection, and methods for cleaving DNA.

A kit as described herein may include one or more containers housing components for performing methods described herein and optionally instructions for use. Any kit described herein may further comprise components needed for performing methods described herein. A component of a kit, where applicable, may be provided in liquid form (e.g., in solution), or in solid form (e.g., a dry powder). In some embodiments, a component of a kit may be reconstituted or otherwise processed (e.g., to an active form), for example, by addition of a suitable buffer, which may or may not be provided with the kit.

In some embodiments, the kit may optionally include instructions and/or promotion for use of the components provided. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure. Additionally, the kit may include other components depending on the specific application, as described herein.

A kit may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kit may include the active agents premixed and shipped in a vial, tube, or other container.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kit can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration, etc.

Pharmaceutical Compositions

The present invention provides pharmaceutical compositions comprising an IdrD protein for use in inhibiting bacterial growth and/or treating a subject.

Pharmaceutical compositions may comprise any suitable IdrD protein as provided herein. In some embodiments, the pharmaceutical composition comprises an IdrD protein variant. In some embodiments, the pharmaceutical composition comprises a fragment of IdrD protein from P. mirabilis. In some embodiments, the pharmaceutical composition comprises a fragment of IdrD protein from Rothia. In some embodiments, the pharmaceutical composition comprises a fragment of IdrD protein from Rothia. In some embodiments, the pharmaceutical composition comprises a mutant of IdrD protein from P. mirabilis. In some embodiments, the pharmaceutical composition comprises a variant of IdrD protein from P. mirabilis. In some embodiments, the pharmaceutical composition comprises a fusion protein comprising a fragment of IdrD protein from P. mirabilis. In some embodiments, the pharmaceutical composition comprises a mutant of IdrD protein from Rothia. In some embodiments, the pharmaceutical composition comprises a variant of IdrD protein from Rothia. In some embodiments, the pharmaceutical composition comprises a fusion protein comprising a fragment of IdrD protein from Rothia. In some embodiments, the fusion protein comprises any of the IdrD proteins, variants, mutants, fragments, or fusion proteins as described herein. In some embodiments, the pharmaceutical composition comprises an IdrD protein with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to a sequence as set forth in any one of SEQ ID NO: 1, 3, 5, 7, 9, 11, or 13. In some embodiments, the pharmaceutical composition comprises an IdrD protein as set forth in any one of SEQ ID NO: 1, 3, 5, 7, 9, 11, or 13. In some embodiments, the pharmaceutical composition comprises an IdrD protein as encoded by a sequence with at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or more, identity to a sequence as set forth in any one of SEQ ID NO: 2, 4, 6, 8, 10, 12, or 14. In some embodiments, the pharmaceutical composition comprises an IdrD protein as encoded by a sequence as set forth in any one of SEQ ID NO: 2, 4, 6, 8, 10, 12, or 14.

In some embodiments, the pharmaceutical composition comprises a nucleic acid encoding a fragment of IdrD protein from P. mirabilis. In some embodiments, the pharmaceutical composition comprises a fragment of IdrD protein from Rothia. In some embodiments, the pharmaceutical composition comprises a nucleic acid encoding a fragment of IdrD protein from Rothia. In some embodiments, the pharmaceutical composition comprises a fusion protein comprising a fragment of IdrD protein from a first bacteria (e.g., P. mirabilis) and a fragment of IdrD protein from a second bacteria (e.g., Rothia).

In some embodiments, the pharmaceutical composition is used for inhibiting bacterial growth. In some embodiments, the pharmaceutical composition is used for treating a disease associated with bacteria trapping neutrophil extracellular traps (NETs). In some embodiments, the pharmaceutical composition is used for treating or preventing a bacterial infection. In some embodiments, the pharmaceutical composition is used for cleaving DNA.

In some embodiments, the IdrD protein is provided in an effective amount in the pharmaceutical composition. In some embodiments, the effective amount is effective for inhibiting bacterial growth. In some embodiments, the effective amount is effective for treating a disease associated with bacteria trapping neutrophil extracellular traps (NETs). In some embodiments, the effective amount is effective for treating or preventing a bacterial infection. In some embodiments, the effective amount is effective for cleaving DNA.

Pharmaceutical compositions described herein may be useful for inhibiting growth of bacteria and/or treating a disease supported by bacteria trapped in NETs. In some embodiments, the bacteria is Proteus mirabilis. In some embodiments, the bacteria is a gram-negative bacteria. Examples of gram-negative bacteria include, but are not limited to, Escherichia, Shigella, Edwardsiella, Salmonella, Citrobacter, Klebsiella, Enterobacter, Hafnia, Serratia, Morganella, Providencia, Yersinia, Erwinia, Buttlauxella, Cedecea, Ewingella, Kluyvera, Tatumella, Rahnella, Pseudomonas aeruginosa, Stenotrophomonas maltophilia, Burkholderia, Cepacia, Gardenerella, Vaginalis, and Acinetobacter species. In some embodiments, the bacteria is a gram-positive bacteria. Examples of gram-negative bacteria include, but are not limited to, Staphylococcus, Streptococci, Enterococci, Corynebacteria, and Bacillus species.

The pharmaceutical compositions described herein may be useful in preventing and/or treating a bacterial infection in a subject. In some embodiments, the bacterial infection is a Proteus mirabilis infection. In some embodiments, the bacterial infection is a gram-negative bacterial infection. Examples of gram-negative bacteria that may infect a subject include, but are not limited to, Escherichia, Shigella, Edwardsiella, Salmonella, Citrobacter, Klebsiella, Enterobacter, Hafnia, Serratia, Morganella, Providencia, Yersinia, Erwinia, Buttlauxella, Cedecea, Ewingella, Kluyvera, Tatumella, Rahnella, Pseudomonas aeruginosa, Stenotrophomonas maltophilia, Burkholderia, Cepacia, Gardenerella, Vaginalis, and Acinetobacter species. In some embodiments, the bacterial infection is a gram-positive bacterial infection. Examples of gram-negative bacteria that may infect a subject include, but are not limited to, Staphylococcus, Streptococci, Enterococci, Corynebacteria, and Bacillus species.

The pharmaceutical compositions described herein may be useful for cleaving DNA. In some embodiments, the DNA is cellular DNA. In some embodiments, the DNA is extracellular DNA. In some embodiments, the DNA is isolated DNA. In some embodiments, the DNA is in a subject. In some embodiments, the DNA is in vitro DNA. In some embodiments, the DNA is in vivo DNA.

It will also be appreciated that the compositions described herein can be employed in combination therapies, that is, the compositions or pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder, or they may achieve different effects (e.g., control of any adverse effects).

In addition to IdrD protein or a fragment thereof, the pharmaceutical composition described herein may comprise one or more additional therapeutic agents. In some embodiments, the therapeutic agent is an antimicrobial including, but not limited to, an antibiotic, an antifungal, or an antiviral. In some embodiments, the therapeutic agent is a chemotherapeutic agent.

Examples of antibiotics include, but are not limited to, penicillins, cephalosporins, carbepenems, other beta-lactams antibiotics, aminoglycosides, macrolides, lincosamides, glycopeptides, tetracylines, chloramphenicol, quinolones, fucidins, sulfonamides, trimethoprims, rifamycins, oxalines, streptogramins, lipopeptides, ketolides, polyenes, azoles, and echinocandins.

Examples of antifungals include, but are not limited to, polyene antifungals—natamycin, rimocidin, filipin, nystatin, amphotericin B, candicin, and hamycin; imidazole antifungals—miconazole, ketoconazole, clotrimazole, econazole, omoconazole, bifonazole, butoconazole, fenticonazole, isoconazole, oxiconazole, sertaconazole, sulconazole, and tioconazole; triazole antifungals—fluconazole, itraconazole, isavuconazole, ravuconazole, posaconazole, voriconazole, terconazole, and albaconazole; thiazole antifungals—abafungin; allylamine antifungals—terbinafine, naftifine, and butenafine; and echinocandin antifungals—anidulafungin, caspofungin, and micafungin. Other compounds that have antifungal properties include, but are not limited to, polygodial, benzoic acid, ciclopirox, tolnaftate, undecylenic acid, flucytosine or 5-fluorocytosine, griseofulvin, and haloprogin.

Examples of antivirals include, but are not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, Foscarnet, Fomivirsen, Ganciclovir, Indinavir, Idoxuridine, Lamivudine, Lopinavir Maraviroc, MK-2048, Nelfinavir, Nevirapine, Penciclovir, Raltegravir, Rilpivirine, Ritonavir, Saquinavir, Stavudine, Tenofovir Trifluridine, Valaciclovir, Valganciclovir, Vidarabine, Ibacitabine, Amantadine, Oseltamivir, Rimantidine, Tipranavir, Zalcitabine, Zanamivir, and Zidovudine.

Examples of chemotherapeutic agents include, but are not limited to, Platinating agents, such as Carboplatin, Oxaliplatin, Cisplatin, Nedaplatin, Satraplatin, Lobaplatin, Triplatin, Tetranitrate, Picoplatin, Prolindac, Aroplatin and other derivatives; Topoisomerase I inhibitors, such as Camptothecin, Topotecan, irinotecan/SN38, rubitecan, Belotecan, and other derivatives; Topoisomerase II inhibitors, such as Etoposide (VP-16), Daunorubicin, a doxorubicin agent (e.g., doxorubicin, doxorubicin HCl, doxorubicin analogs, or doxorubicin and salts or analogs thereof in liposomes), Mitoxantrone, Aclarubicin, Epirubicin, Idarubicin, Amrubicin, Amsacrine, Pirarubicin, Valrubicin, Zorubicin, Teniposide and other derivatives; Antimetabolites, such as Folic family (Methotrexate, Pemetrexed, Raltitrexed, Aminopterin, and relatives); Purine antagonists (Thioguanine, Fludarabine, Cladribine, 6-Mercaptopurine, Pentostatin, clofarabine and relatives) and Pyrimidine antagonists (Cytarabine, Floxuridine, Azacitidine, Tegafur, Carmofur, Capacitabine, Gemcitabine, hydroxyurea, 5-Fluorouracil (5FU), and relatives); alkylating agents, such as Nitrogen mustards (e.g., Cyclophosphamide, Melphalan, Chlorambucil, mechlorethamine, Ifosfamide, mechlorethamine, Trofosfamide, Prednimustine, Bendamustine, Uramustine, Estramustine, and relatives); nitrosoureas (e.g., Carmustine, Lomustine, Semustine, Fotemustine, Nimustine, Ranimustine, Streptozocin, and relatives); Triazenes (e.g., Dacarbazine, Altretamine, Temozolomide, and relatives); Alkyl sulphonates (e.g., Busulfan, Mannosulfan, Treosulfan, and relatives); Procarbazine; Mitobronitol, and Aziridines (e.g., Carboquone, Triaziquone, ThioTEPA, triethylenemalamine, and relatives); Antibiotics, such as Hydroxyurea, Anthracyclines (e.g., doxorubicin agent, daunorubicin, epirubicin and other derivatives); Anthracenediones (e.g., Mitoxantrone and relatives); Streptomyces family (e.g., Bleomycin, Mitomycin C, Actinomycin, Plicamycin); and ultraviolet light.

In some embodiments, the pharmaceutical composition is a solid. In some embodiments, the pharmaceutical composition is a powder. In some embodiments, the pharmaceutical composition can be dissolved in a liquid to make a solution. In some embodiments, the pharmaceutical composition is dissolved in water to make an aqueous solution. In some embodiments, the pharmaceutical composition is a liquid for topical administration (e.g., skin of a subject in need thereof). In some embodiments, the pharmaceutical composition is a liquid for coating a surface (e.g., a tissue). In some embodiments, the pharmaceutical composition is a liquid for oral administration (e.g., ingestion). In some embodiments, the pharmaceutical composition is a liquid for parental injection. In some embodiments, the pharmaceutical composition is a liquid (e.g., aqueous solution) for intravenous injection. In some embodiments, the pharmaceutical composition is a liquid (e.g., aqueous solution) for subcutaneous injection.

After formulation with an appropriate pharmaceutically acceptable excipient in a desired dosage, the pharmaceutical compositions of this invention can be administered to humans and other animals orally, parenterally, intracisternally, intraperitoneally, topically, bucally, or the like, depending on the disease or condition being treated. In some embodiments, a pharmaceutical composition comprising IdrD protein or a fragment thereof is administered, orally or parenterally, at dosage levels of each pharmaceutical composition sufficient to deliver from about 0.001 mg/kg to about 200 mg/kg in one or more dose administrations for one or several days (depending on the mode of administration).

Some aspects of this disclosure provide IdrD protein in a therapeutically effective amount. In some embodiments, a therapeutically effective amount of IdrD protein as provided herein may vary depending upon known factors, such as use and length of use, pharmaceutical characteristics of the composition, and age, sex, weight, and health of the subject. In some embodiments, the therapeutically effective amount of IdrD protein is between 0.1 and 0.5 mg. In some embodiments, the therapeutically effective amount of IdrD protein is between 0.5 and 1 mg. In some embodiments, the therapeutically effective amount of IdrD protein is between 1 and 10 mg.

In some embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In some embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). In some embodiments, each composition described herein is administered at a dose that is below the dose at which the agent causes non-specific effects.

In some embodiments, the pharmaceutical composition is administered at a dose of about 0.001 mg to about 200 mg a day. In some embodiments, the pharmaceutical composition is administered at a dose of about 0.01 mg to about 100 mg a day. In some embodiments, pharmaceutical composition is administered at a dose of about 0.01 mg to about 50 mg a day. In some embodiments, the pharmaceutical composition is administered at a dose of about 0.01 mg to about 10 mg a day. In some embodiments, the pharmaceutical composition is administered at a dose of about 0.1 mg to about 10 mg a day.

Pharmaceutical compositions described herein can be prepared by any method known in the art of pharmacology. In general, such preparatory methods include the steps of bringing the composition comprising IdrD protein, into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include, but are not limited to, pharmaceutically acceptable emulsions, microemulsions, solutions, suspensions, syrups, and elixirs. In addition to the active agents, the liquid dosage forms may contain inert diluents commonly used in the art such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can also include adjuvants such as wetting agents, emulsifying and suspending agents, sweetening, flavoring, and perfuming agents. In certain embodiments for parenteral administration, agents of the invention are mixed with solubilizing agents such CREMOPHOR EL® (polyethoxylated castor oil), alcohols, oils, modified oils, glycols, polysorbates, cyclodextrins, polymers, and combinations thereof.

Injectable preparations, for example, sterile injectable aqueous or oleaginous suspensions, may be formulated according to the known art using suitable dispersing or wetting agents and suspending agents. Sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the active agent is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active agents can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active agent may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Formulations suitable for topical administration include liquid or semi-liquid preparations such as liniments, lotions, gels, applicants, oil-in-water or water-in-oil emulsions such as creams, ointments, or pastes; or solutions or suspensions such as drops. Formulations for topical administration to the skin surface can be prepared by dispersing the drug with a dermatologically acceptable carrier such as a lotion, cream, ointment, or soap. Useful carriers are capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used. Ophthalmic formulation, ear drops, and eye drops are also contemplated as being within the scope of this invention. Additionally, the present invention contemplates the use of transdermal patches, which have the added advantage of providing controlled delivery of an agent to the body. Such dosage forms can be made by dissolving or dispensing the agent in the proper medium. Absorption enhancers can also be used to increase the flux of the agent across the skin. The rate can be controlled by either providing a rate controlling membrane or by dispersing the agent in a polymer matrix or gel.

Additionally, the carrier for a topical formulation can be in the form of a hydroalcoholic system (e.g., quids and gels), an anhydrous oil or silicone based system, or an emulsion system, including, but not limited to, oil-in-water, water-in-oil, water-in-oil-in-water, and oil-in-water-in-silicone emulsions. The emulsions can cover a broad range of consistencies including thin lotions (which can also be suitable for spray or aerosol delivery), creamy lotions, light creams, heavy creams, and the like. The emulsions can also include microemulsion systems. Other suitable topical carriers include anhydrous solids and semisolids (such as gels and sticks); and aqueous based mousse systems.

Methods of Coating a Surface

In one aspect, the present disclosure provides IdrD protein for use in inhibiting or preventing bacterial growth on a surface. In some embodiments, the IdrD protein is coated onto a surface that may be prone to bacterial contamination. In some embodiments, the IdrD protein is coated onto a surface contaminated by a bacteria. In some embodiments, the IdrD protein is applied prophylactically over a “clean” surface that is not contaminated by bacteria. In some embodiments, the proteins of the present disclosure are used to prevent a biofilm on a surface or composition. In some embodiments, the proteins of the present disclosure are used to degrade a biofilm already present on a surface or composition. In some embodiments, the biofilm being treated, degraded, prevented, or inhibited is the result of an infection, or from exposure to, at least in part, Staphylococcus epidermis. In some embodiments, the biofilm being treated, degraded, prevented, or inhibited is the result of an infection, or from exposure to, at least in part, Staphylococcus aureus. In some embodiments, the biofilm being treated, degraded, prevented, or inhibited is the result of an infection, or from exposure to, at least in part, Pseudomonas aeruginosa.

In some embodiments, the IdrD protein is coated onto a surface. In some embodiments, the surface is a tissue. In some embodiments, the tissue is selected from the group consisting of skin tissue, tumor tissue, and organ tissue. In some embodiments, the tissue is a skin tissue. In some embodiments, the tissue is a tumor tissue. In some embodiments, the tissue is an organ tissue. In some embodiments, the tissue is an infected tissue. In some embodiments, the tissue is a wounded tissue. In some embodiments, the tissue has a biofilm present. In some embodiments, the tissue does not have a biofilm present. In some embodiments, the proteins of the present disclosure are used in combination with a skin graft. In some embodiments, the proteins of the present disclosure are applied with or directly to a wound dressing. In some embodiments, the proteins of the present disclosure are used in combination with, or applied directly to, a medical device, prosthesis, or therapy device.

In some embodiments, the wounded tissue is an ulcer. In some embodiments, the wounded tissue is the result of a chronic wound. In some embodiments, the chronic wound is an ulcer. In some embodiments, the ulcer or chronic wound is a diabetic wound. In some embodiments, the diabetic wound is a diabetic ulcer.

In some embodiments, the IdrD protein is coated onto a surface of a medical device. Exemplary medical devices include, but are not limited to, catheters such as urinary catheters, venous catheters, arterial catheters, dialysis catheters, peritoneal catheters, urinary sphincters, urinary dilators, urinary stents, tissue bonding urinary devices, vascular grafts, vascular dilators, extravascular dilators, vascular stents, extravascular stents, wound drain tubes, shunts, pacemaker systems, joint replacements, heart valves, cardiac assist valves, bone prosthesis, joint prosthesis, or dental prosthesis.

The area of a surface coated by IdrD protein should be sufficient for inhibiting bacterial growth. In some embodiments, the IdrD protein is coated on at least a part of the surface. In some embodiments, the IdrD protein is coated on at least 1%, at least 5%, at least 10%, at least 20%, at least 30%, at least 40%, at least 50%, at least 60%, at least 70%, at least 80%, at least 90%, or at least 99.5% of the surface.

Any suitable IdrD protein for use in inhibiting bacterial growth on a surface may be coated onto the surface. In some embodiments, the IdrD protein is in the form of a liquid. In some embodiments, the IdrD protein is in the form of a powder. In some embodiments, the IdrD protein can be dissolved in a liquid to make a solution. In some embodiments, the IdrD protein-containing liquid is a solution, a suspension, a colloid, or a dispersion.

Any method suitable for coating IdrD protein onto a surface may be used. In some embodiments, coating IdrD protein onto a surface comprises spraying IdrD protein onto the surface. In some embodiments, coating IdrD protein onto a surface comprises brushing IdrD protein onto the surface. In some embodiments, coating IdrD protein onto a surface comprises applying IdrD protein onto the surface. In some embodiments, coating IdrD protein onto a surface comprises coating drD protein onto the surface.

Any amount of IdrD protein suitable for inhibiting bacterial growth and/or swarming on a surface may be used. In some embodiments, the surface has between 0.1 and 1.0 mg of IdrD protein per cm² of surface area. In some embodiments, the surface has between 1 and 10 mg of IdrD protein per cm² of surface area. In some embodiments, the surface has between 10 and 100 mg of IdrD protein per cm² of surface area.

Any thickness of IdrD protein coating that does not altering the functionality of a surface may be coated onto the surface. In some embodiments, the IdrD protein coating is between about 0.0001 millimeters and 10 millimeters in thickness. In some embodiments, the IdrD protein coating is between 0.5 and about 5 millimeters in thickness. In some embodiments, the IdrD protein coating is between 1 and about 4 millimeters in thickness.

Methods of Cleaving DNA

In one aspect, the present disclosure provides IdrD protein for use in cleaving DNA. In some embodiments, methods comprise incubating DNA and an effective amount of IdrD protein. In some embodiments, the DNA is obtained or is part of a cell lysate or a cell culture. In some embodiments, the sample comprising DNA is obtained from an in vitro reaction mixture. In some embodiments, the sample comprising DNA is obtained from a subject (e.g., a patient).

Any suitable IdrD protein may be used for cleaving DNA described herein. In some embodiments, the IdrD protein is a liquid. In some embodiments, the IdrD protein is a powder. In some embodiments, the IdrD protein is dissolved in a liquid to make a solution. In some embodiments, the IdrD protein-containing liquid is a solution, a suspension, a colloid, or a dispersion.

Any temperature may be used in a method of cleaving DNA described herein. In some embodiments, methods of cleaving DNA comprise incubating DNA with an effective amount of IdrD protein at a temperature of 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., or 42° C.

Any incubation time may be used in a method of cleaving DNA described herein. For example, methods of cleaving DNA comprise incubating DNA and IdrD protein for at least 0.25, 0.5, 0.75, 1, 2, 3, 4, 5, 6, 7, 8, 9, 10, 11, 12, 13, 14, 15, 16, 17, 18, 19, 20, 24, 36, 42, or 48 hours.

Any amount of DNA may be cleaved in a method of cleaving DNA described herein. For example, methods of cleaving DNA comprise cleaving at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of DNA.

Methods of Preventing and/or Treating a Bacterial Infection

In one aspect, the present disclosure provides methods for preventing and treating bacterial infections in a subject using IdrD protein. In some embodiments, the bacterial infection is a Proteus mirabilis infection. In some embodiments, the bacterial infection is caused by a Gram-positive bacterium. Exemplary Gram-positive bacteria include, but are not limited to, species of the genera Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, and Corynebacterium. In some embodiments, the Gram-positive bacterium is a bacterium of the phylum Firmicutes. In some embodiments, the bacterium is a member of the phylum Firmicutes and the genus Enterococcus, e.g., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus. In some embodiments, the Enterococcus infection is an E. faecalis infection. In some embodiments, the Enterococcus infection is an E. faecium infection. In some embodiments, the bacteria is a member of the phylum Firmicutes and the genus Staphylococcus, e.g., the bacterial infection is a Staphylococcus infection. Exemplary Staphylococci bacteria include, but are not limited to, S. arlettae, S. aureus, S. auricularis, S. capitis, S. caprae, S. carnous, S. chromogenes, S. cohii, S. condimenti, S. croceolyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. lenus, S. lugdunesis, S. lutrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus. In some embodiments, the Staphylococcus infection is an S. aureus infection. In some embodiments, the Staphylococcus infection is an S. epidermis infection. In some embodiments, the Gram-positive bacterium is selected from the group consisting of Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae, Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes, Streptococcus salivarius, Streptococcus sanguis, Bacillus anthracis, Clostridium botulinum, Clostridium perfringens, Clostridium tetani, Corynebacterium diphtheriae, Corynebacterium jeikeium, Enterococcus faecalis, Enterococcus faecium, Erysipelothrix rhusiopathiae, Gardnerella vaginalis, Gemella morbillorum, Mycobacterium abcessus, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, and Peptococcus niger.

In some embodiments, the bacterial infection being treated and/or prevented is an infection caused by a Gram-negative bacterium. Exemplary Gram-negative bacteria include, but are not limited to, Escherichia coli, Caulobacter crescentus, Pseudomonas, Agrobacterium tumefaciens, Branhamella catarrhalis, Citrobacter diversus, Enterobacter aerogenes, Klebsiella pneumoniae, Proteus mirabilis, Salmonella typhimurium, Neisseria meningitidis, Serratia marcescens, Shigella sonnei, Neisseria gonorrhoeae, Acinetobacter baumannii, Salmonella enteriditis, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Morganella morganii, Edwardsiella tarda, and Haemophilus influenzae. In certain embodiments, the Gram-negative bacteria species is Pseudomonas. In certain embodiments, the Gram-negative bacteria species is Pseudomonas aeruginosa.

In some embodiments, the bacterial infection being treated and/or prevented is a urinary tract infection (most commonly caused by Escherichia coli, Proteus mirabilis, and/or Staphylococcus saprophyticus). In some embodiments, the bacterial infection is a catheter-associated urinary tract infection. In some embodiments, the bacterial infection is gastritis (most commonly caused by Helicobacter pylori), respiratory infection (such as those commonly afflicting patents with cystic fibrosis, most commonly caused by Pseudomonas aeruginosa), cystitis (most commonly caused by Escherichia coli), pyelonephritis (most commonly caused by Proteus species, Escherichia coli and/or Pseudomonas sp), osteomyelitis (most commonly caused by Staphylococcus aureus, but also by Escherichia coli), bacteremia, skin infection, rosacea, acne, chronic wound infection, infectious kidney stones (can be caused by Proteus mirabilis), bacterial endocarditis, and/or sinus infection.

In some embodiments, the bacterial infection being treated and/or prevented is caused by an organism resistant to one or more antibiotics. For example, in some embodiments, the bacterial infection is caused by an organism resistant to penicillin. In some embodiments, the bacterial infection is caused by an organism resistant to vancomycin (VR). In some embodiments, the bacterial infection is caused by vancomycin-resistant E. faecalis. In some embodiments, the bacterial infection is caused by vancomycin-resistant E. faecium. In some embodiments, the bacterial infection is caused by vancomycin-resistant Staphylococcus aureus (VRSA). In some embodiments, the bacterial infection is caused by vancomycin-resistant Enterococci (VRE). In some embodiments, the bacterial infection is caused by a methicillin-resistant (MR) organism. In some embodiments, the bacterial infection is caused by methicillin-resistant S. aureus (MRSA). In some embodiments, the bacterial infection is caused by methicillin-resistant Staphylococcus epidermidis (MRSE). In some embodiments, the bacterial infection is caused by penicillin-resistant Streptococcus pneumonia. In some embodiments, the bacterial infection is caused by quinolone-resistant Staphylococcus aureus (QRSA). In some embodiments, the bacterial infection is caused by multi-drug resistant Mycobacterium tuberculosis.

Methods of Treating a Disease Associated with Bacteria Trapping Neutrophil Extracellular Traps (NETs)

Bacteria trapping neutrophil extracellular traps (NETs) refer to networks of extracellular fibers composed primarily of DNA that bind bacteria. Without being bound by theory, aberrant formation of bacteria trapping NETs may be important in the generation of a wide range of diseases, including cancer, autoimmune diseases, and inflammatory diseases. Accordingly, in some embodiments, the present disclosure provides methods for treating a disease associated with bacteria trapping neutrophil extracellular traps (NETs) in a subject using IdrD protein.

A variety of diseases may be associated with bacteria trapping NETs. In some embodiments, the disease associated with bacteria trapping NETs is selected from the group consisting of a wound, an infection, a lung disease, a cancer, and a vascular disease. In some embodiments, the disease associated with bacteria trapping NETs is a wound. In some embodiments, the disease associated with bacteria trapping NETs is an infection (e.g., a bacterial infection). In some embodiments, the disease associated with bacteria trapping NETs is a lung disease. In some embodiments, the lung disease is cystic fibrosis. In some embodiments, the disease associated with bacteria trapping NETs is a cancer. In some embodiments, the disease associated with bacteria trapping NETs is a vascular disease. In some embodiments, the vascular disease is thrombosis. In some embodiments, the disease associated with bacteria trapping NETs is an autoimmune disease. In some embodiments, the autoimmune disease is vasculitis. In some embodiments, the autoimmune disease is systemic lupus erythematosus (SLE). In some embodiments, the disease associated with bacteria trapping NETs is an inflammatory disease. In some embodiments, the inflammatory disease is psoriasis.

Bacteria trapping NETs may comprise any bacteria. In some embodiments, the bacteria trapping NETs comprise Proteus mirabilis. In some embodiments, the bacteria trapping NETs comprises a Gram-positive bacterium. Exemplary Gram-positive bacteria include, but are not limited to, species of the genera Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, and Corynebacterium. In some embodiments, the Gram-positive bacterium is a bacterium of the phylum Firmicutes. In some embodiments, the bacterium is a member of the phylum Firmicutes and the genus Enterococcus, e.g., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus. In some embodiments, the Enterococcus infection is an E. faecalis infection. In some embodiments, the Enterococcus infection is an E. faecium infection. In some embodiments, the bacteria is a member of the phylum Firmicutes and the genus Staphylococcus, e.g., the bacterial infection is a Staphylococcus infection. Exemplary Staphylococci bacteria include, but are not limited to, S. arlettae, S. aureus, S. auricularis, S. capitis, S. caprae, S. carnous, S. chromogenes, S. cohii, S. condimenti, S. croceolyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. lenus, S. lugdunesis, S. lutrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus. In some embodiments, the Staphylococcus infection is an S. aureus infection. In some embodiments, the Staphylococcus infection is an S. epidermis infection. In some embodiments, the Gram-positive bacterium is selected from the group consisting of Staphylococcus aureus, Staphylococcus auricularis, Staphylococcus capitis, Staphylococcus cohnii, Staphylococcus epidermidis, Staphylococcus haemolyticus, Staphylococcus hominis, Staphylococcus lugdanensis, Staphylococcus saccharolyticus, Staphylococcus saprophyticus, Staphylococcus schleiferi, Staphylococcus similans, Staphylococcus warneri, Staphylococcus xylosus, Streptococcus agalactiae, Streptococcus anginosus, Streptococcus bovis, Streptococcus canis, Streptococcus equi, Streptococcus milleri, Streptococcus mitior, Streptococcus mutans, Streptococcus pneumoniae, Streptococcus pyogenes, Streptococcus salivarius, Streptococcus sanguis, Bacillus anthracis, Clostridium botulinum, Clostridium perfringens, Clostridium tetani, Corynebacterium diphtheriae, Corynebacterium jeikeium, Enterococcus faecalis, Enterococcus faecium, Erysipelothrix rhusiopathiae, Gardnerella vaginalis, Gemella morbillorum, Mycobacterium abcessus, Mycobacterium chelonae, Mycobacterium fortuitum, Mycobacterium haemophilium, Mycobacterium kansasii, Mycobacterium leprae, Mycobacterium marinum, Mycobacterium scrofulaceum, Mycobacterium smegmatis, Mycobacterium terrae, Mycobacterium tuberculosis, Mycobacterium ulcerans, and Peptococcus niger.

In some embodiments, the bacteria trapping NETs comprise a Gram-negative bacterium. Exemplary Gram-negative bacteria include, but are not limited to, Escherichia coli, Caulobacter crescentus, Pseudomonas, Agrobacterium tumefaciens, Branhamella catarrhalis, Citrobacter diversus, Enterobacter aerogenes, Klebsiella pneumoniae, Proteus mirabilis, Salmonella typhimurium, Neisseria meningitidis, Serratia marcescens, Shigella sonnei, Neisseria gonorrhoeae, Acinetobacter baumannii, Salmonella enteriditis, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Morganella morganii, Edwardsiella tarda, and Haemophilus influenzae. In certain embodiments, the Gram-negative bacteria species is Pseudomonas. In certain embodiments, the Gram-negative bacteria species is Pseudomonas aeruginosa.

In some embodiments, the bacteria trapping NETs comprise an organism resistant to one or more antibiotics. For example, in some embodiments, the bacteria trapping NETs comprise an organism resistant to penicillin. In some embodiments, the bacteria trapping NETs comprise an organism resistant to vancomycin (VR). In some embodiments, the bacteria trapping NETs comprise vancomycin-resistant E. faecalis. In some embodiments, the bacteria trapping NETs comprise vancomycin-resistant Staphylococcus aureus (VRSA). In some embodiments, the bacteria trapping NETs comprise vancomycin-resistant Enterococci (VRE). In some embodiments, the bacteria trapping NETs comprise a methicillin-resistant (MR) organism. In some embodiments, the bacterial infection is caused by methicillin-resistant S. aureus (MRSA). In some embodiments, the bacteria trapping NETs comprise methicillin-resistant Staphylococcus epidermidis (MRSE). In some embodiments, the bacteria trapping NETs comprise penicillin-resistant Streptococcus pneumonia. In some embodiments, the bacteria trapping NETs comprise quinolone-resistant Staphylococcus aureus (QRSA). In some embodiments, the bacteria trapping NETs comprise multi-drug resistant Mycobacterium tuberculosis.

Administration

In some embodiments, the subject administered the IdrD protein or pharmaceutical composition as provided herein is a mammal. In some embodiments, the subject is a human. In certain embodiments, the subject is an immunocompromised subject. In some embodiments, the subject is a domesticated animal, such as a dog, cat, cow, pig, horse, sheep, or goat. In some embodiments, the subject is a companion animal such as a dog or cat. In some embodiments, the subject is a livestock animal such as a cow, pig, horse, sheep, or goat. In some embodiments, the subject is a zoo animal. In another embodiment, the subject is an experimental animal, such as a rodent or non-human primate.

In some embodiments, the IdrD protein or pharmaceutical composition as provided herein may be administered by any administration route known in the art. For example, in some embodiments, one of ordinary skill in the art of medicine, can administer the agents via conventional routes, e.g., administered orally, parenterally, by inhalation spray, topically, rectally, nasally, buccally, vaginally or via an implanted reservoir. In some embodiments, the route of administration is topically to a surface. In some embodiments, the route of administration is topically to a tissue. In some embodiments, the IdrD protein or pharmaceutical composition is formulated for topical administration.

IdrD protein or pharmaceutical composition comprising IdrD protein can be administered concurrently with, prior to, or subsequent to, one or more additional therapeutically active agents (e.g., antibiotics, anti-inflammatory agents). In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

In some embodiments, the subject is administered IdrD protein and one or more therapeutic agents. In some embodiments, the therapeutic agent is an antimicrobial including, but not limited to, an antibiotic, an antifungal, or an antiviral. In some embodiments, the therapeutic agent is a chemotherapeutic agent. In some embodiments, the therapeutic agent is an anti-inflammatory agent. Examples of an antibiotic, an antifungal, an antiviral, a chemotherapeutic agent, and an anti-inflammatory agent are provided herein.

### EXAMPLES

The following Examples are intended to illustrate and to describe particular embodiments, but are not intended to exemplify the full scope of the invention. Accordingly, it will be understood that the Examples are not meant to limit the scope of the invention.

Introduction

Bacteria within communities compete for resources. One strategy is to inject lethal effectors into adjacent cells; clonal siblings survive by neutralizing the effector while all other cells die [6, 8, 21]. Such bacterial effectors, which can act in many ways such as disrupt cell membranes and degrade proteins or nucleic acid [23], are particularly useful for bacteria in gut microbiomes and other mixed communities. Individual strains of a single species can encode different effectors, each with different structures [7, 53, 54]. Abundant species use their specific effectors to dominate in a mixed-species community [5, 42]. Yet low-abundance bacteria can remain successful residents within mixed-species communities that have a dominant strain; this mechanism for survival remains unclear. Moreover, strain-specific proteins could provide a genome-encoded mechanism to differentiate among closely related bacteria.

The swarming bacterium Proteus mirabilis, a human opportunistic pathogen, is one of the low-abundance bacteria in some human guts. In P. mirabilis, the gene IdrD, encoding a contact-dependent lethal effector, was identified in a screen for self versus nonself recognition [5]. Strains lacking a functional IdrD gene are unable to merge colonies with their wild-type parent or dominate in mixed migrating populations [5] and possibly in the human host [55]. IdrD is exported by a Type VI secretion system, allowing for transport through direct physical contact with adjacent cells [5, 17]. The C-terminal end of IdrD encodes a potential cell-contact dependent effector protein, IdrD-CT.

Polymorphic effectors like IdrD contain regions with known functions: the N-terminal domain aids in the export via Types IV, V, or VI secretion systems [20-22] and the C-terminal domain often contains the lethal effector [6-8, 19]. For example, the well-established polymorphic effectors RhsA and RhsB have their enzymatic nuclease domains at the C-terminus; 3 putative proteolytic cleavage results in the smaller effectors, RhsA-CT and RhsB-CT, respectively [6, 56]. Interestingly, effectors with similar functions, such as degrading DNA, can be of different protein families, each with distinct secondary and tertiary structures. This domain architecture could promote the existence of species-identifying effectors, leading to a hypothesis that the effector, encoded within the entire C-terminal domain, encodes specificity for a species [57]. However, we found that proteins with similarity to IdrD-CT contained more sequence variation than could be explained by this hypothesis.

The uncharacterized IdrD-CT protein was particularly interesting because secondary and tertiary structure analysis predicted the presence of a PD-(D/E)XK motif, which is found in a broad superfamily of nucleases. Contact-dependent effectors in Burkholderia pseudomallei that degrade tRNA [27] are an example of such nucleotide-targeting enzymes. Database searches revealed additional proteins with similarity to IdrD-CT, yet none of these proteins belonged to known families of the PD-(D/E)XK nuclease superfamily. Even though proteins containing the PD-(D/E)XK motif are found in animals, plants, and bacteria [24], predicting the nucleotide target from the amino acid sequences remains difficult. Finding and characterizing additional families is critical for developing algorithms that could predict function from sequence.

As such, we sought to characterize the function and nucleotide target of the IdrD-CT protein. Through biochemical assays, it was found that IdrD-CT, and proteins with similar sequences, form a distinct family of PD-(D/E)XK nuclease proteins. It was next investigated whether one could differentiate between low and high abundance bacteria in human datasets by adapting metagenomics methods to find IdrD-CT. Metagenomes are short-read DNA datasets generated from all bacteria within a sampled community or microbiome. An adapted metagenomic analysis revealed that members of the IdrD-CT protein family are rare in human oral and gut microbiomes, even though the full-length encoding gene, IdrD, is not. Analysis of metagenomes also showed that IdrD-CT proteins have an architecture with two separate domains, one encoding deoxyribonuclease (DNase) activity and the other with species-identifying sequences. IdrD-CT has a conserved cleavage action in the DNase domain and is flexible to amino acid changes, including entire domain replacements, in the species-identifying domain. Species-identifying effectors could provide a means to differentiate among bacterial species within gut and oral microbiome datasets. The domain architecture of the IdrD-CT protein and related proteins raise questions about how strain-specific effectors evolve and how bacteria might deploy these effectors for survival at low abundance.

### Example 1: Materials and Methods

Bacterial strains and media. Overnight cultures of all strains were grown aerobically at 37° C. in LB broth. Swarm-permissive nutrient plates were made with CM55 blood agar base agar (Oxoid, Basing-stoke, England). P. mirabilis strains were maintained on low swarming agar (LSW−). All swarm and growth media contained 35 g/ml kanamycin for plasmid maintenance.

Strain construction. The plasmid P_{idrA}-IdrD-CT was constructed by PCR amplifying the last 416 bp of the IdrD gene from BB2000 using primers AS174 and AS175 and cloning it into the SacI and AgeI sites of pAS1034, resulting in plasmid pAS1054. The inducible anhydrotetracycline promoter (Ptet) was introduced into the IdrD-CT expression vectors by generating gBlocks (gDS0005) of the promoter region with 29 bp overhangs for the plasmid, and using SLiCE to recombine into pAS1054. This resulted in the plasmid pDS0002 (producing IdrD-CT).

A C-terminal FLAG tag (GACTACAAGGACGACGATGACAAG (SEQ ID NO: 16)) was added to IdrD by using SLiCE to recombine the gBlock gDS0023 (FLAG tag with 49 bp overhang of IdrD-CT and 52 bp overhang of pDS0002) into pDS0002. This vector is pDS0034. The FLAG-tagged IdrD active site mutants were generated by replacing the gene encoding IdrD-CT-FLAG in pDS0034 with the mutants sequences which are encoded in gBlocks gDS0025-28-resulting in plasmids pDS0048 (D1482A), pDS0049 (E1496A), pDS0050 (K1498A), and pDS0051 (triple mutant). The untagged versions of IdrD were constructed by PCR amplifying the mutant IdrD sequences from pDS0048-51 with oDS0137 and oDS0159 to remove FLAG tag, and performing a restriction digest with SacI and AgeI to insert into pDS0034 (pDS0058-61).

To generate pDS0062, a gfp expression vector under the control of Ptet, primers oDS0161 and oDS0162 were used to amplify gfpmut2 from pidsBB-idsE-GFP, and put into pDS0034 through restriction digest with SacI and AgeI.

All plasmids were confirmed by Sanger sequencing (Genewiz). Plasmids with conjugative transfer elements, including all Idr expression vectors, were moved into the Escherichia coli conjugative strain S17 which were then mated with recipient P. mirabilis strains as previously described. The presence of plasmids was confirmed in recipient strains by PCR using plasmid-specific primers.

Swarm viability assay. Overnight cultures of BB2000 IdrD* carrying each expression vector were normalized to an optical density at 600 nM (OD₆₀₀) of 1; swarm-permissive nutrient plates supplemented with kanamycin and 10 nM anhydrotetraclycine were inoculated with 1 uL of normalized culture. Plates were incubated for 48 hours at room temperature. Swarms were then resuspended in 6 ml of LB broth; 20 uL of this resuspension was used for a 10-fold dilution series (total of 8 dilutions). 10 uL of each dilution was spotted onto LSW-agar plates supplemented with kanamycin.

Liquid viability assay. Overnight cultures of E. coli MG1655 carrying each expression vector were normalized to an optical density at 600 nM (OD₆₀₀) of 1.2 μl of normalized cultures was added to 198 μl of LB broth supplemented with kanamycin and 10 nM anhydrotetracycline and grown at 37° C. for 16 hours in a 96 well-plate. OD₅₉₅ reading were taken every half hour.

In vitro DNase assay. IdrD-CT and IdrD-CT_{D39A} with a C-terminal FLAG epitope tag were produced using the New England Biolabs PURExpress In Vitro Protein Synthesis Kit. Template DNA was amplified from pDS0034 (producing IdrD-CT-FLAG) or pDS0048 (producing IdrD-CT_{D39A}-FLAG) using primers with overhangs to add the required elements specified by the PURExpress kit. Reactions were performed with 250 ng of template DNA (no template DNA added to negative control reaction) and incubated at 37° C. for two hours. Protein amount was determined using an α-FLAG western blot with a known gradient of FLAG-BAP (2.5, 5, 10, 20 ng). Protein (2.5, 5, and 10 ng) was added to 0.5 μg of lambda DNA (methylated and unmethylated), 5 μL of New England Biolabs Buffer 3.1, and up to a final volume of 25 μL. For plasmid DNase assays, 10 ng of protein was added to 250 ng of circular or linear plasmid DNA (pidsBB). This reaction was incubated for one hour at 37° C., then Proteinase K (New England Biolabs) was added and incubated for 15 minutes at 37° C. Reaction was then run on a 1% agarose gel for analysis.

Western Blotting. In vitro translation reaction samples described herein were run on a 12% Tris-Tricine polyacrylamide gel, transferred to a nitrocellulose membrane, probed with rabbit anti-FLAG (1:4,000; Sigma-Aldrich, St. Louis, Mo.), then goat anti-rabbit conjugated to horseradish peroxidase (HRP) (1:5,000; KPL, Inc., Gaithersburg, Md.), and finally developed using Immun-Star HRP substrate kit (Bio-Rad Laboratories, Hercules, Calif.). Visualization of blots were done using a Chemidoc (Bio-Rad Laboratories, Hercules, Calif.) and TIFF files were used for analysis on Fiji (ImageJ, Madison, Wis.).

Phylogenetic reconstruction of IdrD and species relationships. The amino acid sequences for the 25 genes encoding IdrD-CT identified as described above were obtained and aligned with muscle [43] before removing positions with less than 70% occupancy with trimAl [44]. This alignment was passed to MrBayes v3.2.6 [45] to reconstruct the tree using the WAG substitution model [46] and gamma model of rate heterogeneity. Four independent runs, each run for 20 million generations sampled every 1,000 generations by four coupled chains heated at the default temperature of 0.2, were checked for convergence and then combined into a 50% majority rule tree after burning the initial 40% of the sampled trees.

The species tree was reconstructed using identical methods but with full-length 16S ribosomal RNA sequences obtained from the various genomes, choosing arbitrarily if multiple 16S rRNA copies were found in a genome. Specifically, alignment and MrBayes parameters were identical except for changing the model to GTR [47].

Maximum-likelihood trees were also generated with RAxML [48] using the appropriate WAG or GTR+gamma models and produced identical topologies.

Metagenome selection and processing. A fully-reproducible workflow explaining and documenting all commands and scripts used to perform the metagenome analyses is available. Full-length IdrD nucleotide sequences were obtained from Acinetobacter baumannii XH858, Cronobacter turicensis z3032, Prevotella jejuni CD3:33, Proteus mirabilis BB2000, Pseudomonasfluorescens F113, Rothia aeria C6B, and Xanthomonas citri pv. malvacearum XcmN1003. Publically-available metagenomes likely to represent populations with these genes were identified using the Search SRA portal <www.searchsra.org> that employs the PARTIE algorithm [34]. Briefly, PARTIE uses bowtie2 [49] to search a 100,000-read random subset from each of ˜110,000 published metagenomes. From this, 3,801 candidate metagenomes were identified that contributed non-zero coverage to at least one IdrD sequence, from which we curated a list of 1,189 metagenomes by removing genome assemblies, transcriptomes, non-random library preparation methods, etc. 1,188 of these metagenomes were downloaded from available on NCBI's Short Read Archive (fastq-dump--split-3) for deeper analysis (1 metagenome could not be downloaded). A single bowtie2 database was created with all seven IdrD nucleotide sequences, onto which bowtie2 mapped metagenomes using default parameters (--sensitive; [49]). Anvi'o, an analysis and visualization platform for ‘omics data, managed the resultant data and subsequent analyses [50]. With Anvi'o, a contigs database was generated (anvi-gen-contigs-db command) from the seven IdrD sequences and profiled with the merged results of the bowtie2 mapping (anvi-profile and anvi-merge commands, respectively). Per-nucleotide coverages and variability (e.g., single-nucleotide polymorphism (SNP) counts) were exported for all sequences with the Anvi'o anvi-get-split-coverages and anvi-gen-variability-profile commands, respectively.

To investigate the distribution of IdrD coverage among different Prevotella in the human oral cavity, IdrD sequences from the three additional Prevotella spp. (P. sp. C561, P. fusca JCM 17724, P. denticola NCTC 13067) along with the P. jejuni CD3:33 IdrD sequence were mapped separately, using the same methods but mapping reads from only the 202 metagenomes from the human oral cavity.

Metagenome categorization. Metagenomes were binned into categories based on the provided “ScientificName” annotation. Metagenomes from the Human Microbiome Project (HMP) were listed as “human metagenome”; these were disaggregated into “HMP oral metagenome” and “HMP gut metagenome” based on the sampled site listed in the “Analyte_Type” column. From the 1,188 metagenomes, we focused on only the 324 human oral or human gut metagenomes, defining human oral as metagenomes labelled “human oral metagenome”, “HMP oral metagenome”, or “Non-HMP oral metagenome” (n=42) and human gut as metagenomes labelled “human gut metagenome” or “human metagenome” (n=277). Metagenomes annotated as “human metagenome” (n=47) were categorized as human gut since though they originated from a variety of human body sites, the only three passing the filtration criteria (see next section) were from the human gut. Metagenomes annotated as “oral metagenome” (n=112) were excluded as they provided extremely low (single digit), noisy coverages. In addition, five metagenomes were specifically discarded: SRR628272 was removed from all datasets as the original FASTQ had extremely low quality scores; SRR1779144 came from a diseased infant and skewed the y-axis with an extremely high C. turicensis coverage (>1,000×); SRR2047620 came from a pediatric stem-cell treatment dataset with high antibiotic loads with similarly extreme C. turicensis coverage; SRR1781983 came from a subgingival plaque of a patient with periodontitis and had extremely high R. aeria coverage that skewed the y-axis (>500×); and SRR1038387 came from an infant with necrotizing enterocolitis with extremely high P. mirabilis coverage that skewed the y-axis (>2,000×). 1,183 metagenomes remained after discarding these metagenomes, of which 319 were human oral or human gut metagenomes. Moving forward, we focused exclusively on P. mirabilis, R. aeria, C. turicensis, and P. jejuni as the other taxa's IdrD sequences had little to no coverage from the human metagenomes of interest.

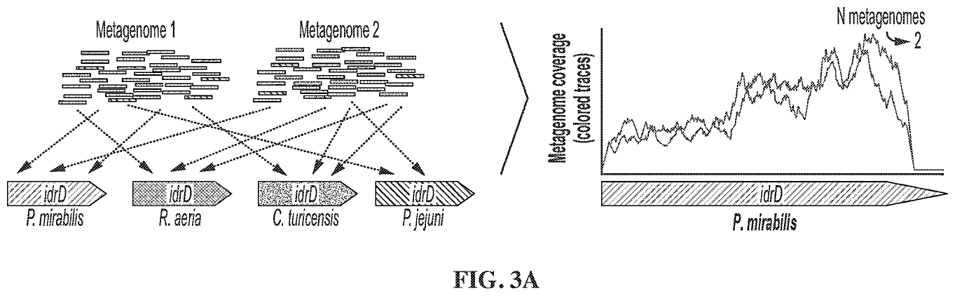

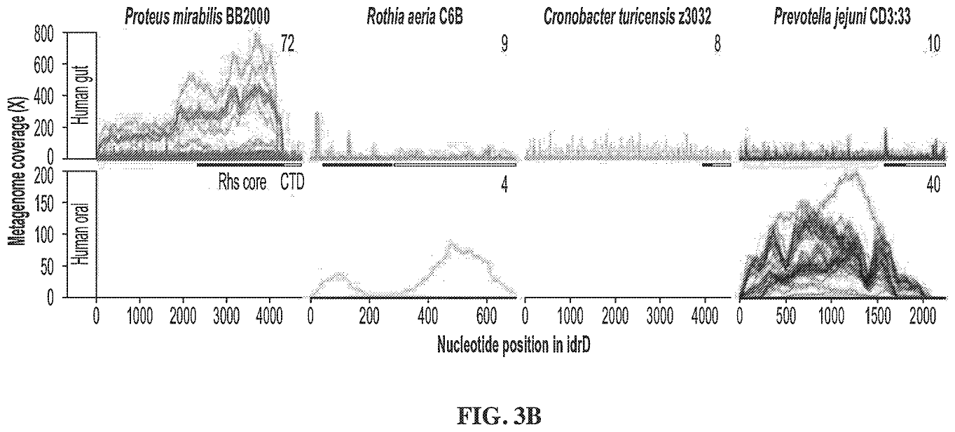

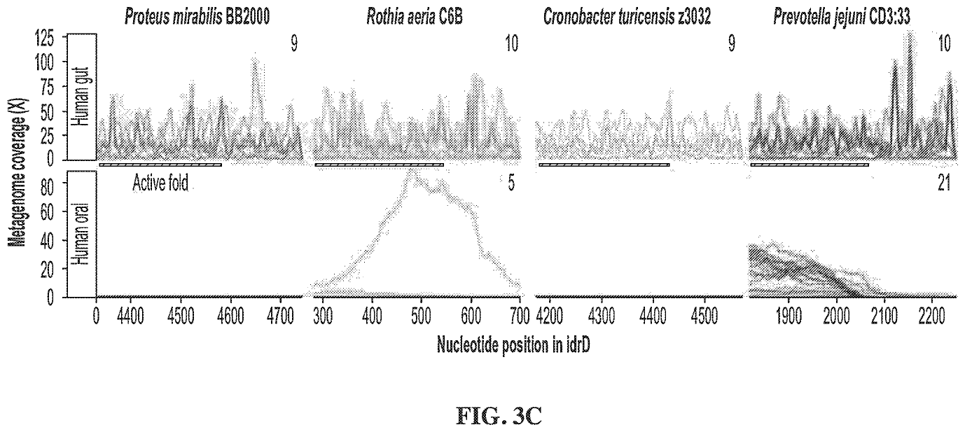

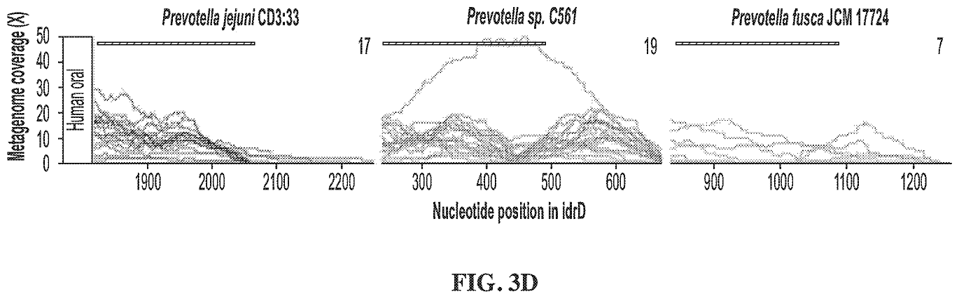

Mapping filtration criteria. To minimize noisy coverage originating from metagenomes relevant for one organism but not another, we employed a filtering strategy that, for each IdrD sequence, considered only metagenomes from which at least half of the nucleotides received coverage. For FIGS. 3C and 3D, the filtering criterion was applied after subsetting to the C-terminal domain (CTD); that is, the filtration was applied based only on the CTD. The number of metagenomes passing the criterion is displayed in the top right corner of each subpanel in FIG. 3. For each metagenome passing the filtration step, each nucleotides' coverage is plotted as a partially-transparent line; thus, metagenomes with similar coverage trends overlap and appear darker.

SNP information from the metagenomes, relative to the seven IdrD reference sequences, is displayed by vertical bars showing the Shannon Entropy [51] of the frequencies of each nucleotide across the metagenomes. Higher entropy values correspond to more even SNP diversity, while an entropy of 0 signifies an invariant position. Entropy values were calculated using the observed nucleotide frequencies found in all metagenomic reads mapping to a given nucleotide position.

TABLE 1
shows strains used in experimental studies described herein.

| Strain | Notes | KAG # | DS # | Source |
|---|---|---|---|---|
| P. mirabilis | IdrD:: Tn-Cm(R) | 3277 | 349 | Provided |
| BB2000 IdrD* + | producing GFPmut2 | | | herein |
| pDS0062 | under the control of | | | |
| | an anhydrotetracyline- | | | |
| | inducible promoter | | | |
| | (pBBR1 origin, Kan | | | |
| | (R)) | | | |
| P. mirabilis | IdrD:: Tn-Cm(R) | 2178 | 104 | Provided |
| BB2000 IdrD* + | producing IdrD-CT | | | herein |
| pDS0002 | under the control of | | | |
| | an anhydrotetracyline- | | | |
| | inducible promoter | | | |
| | (pBBR1 origin, Kan | | | |
| | (R)) | | | |
| P. mirabilis | IdrD:: Tn-Cm(R) | 3236 | 344 | Provided |
| BB2000 IdrD* + | producing IdrD- | | | herein |
| pDS0058 | CT_{D39A} under the | | | |
| | control of an | | | |
| | anhydrotetracyline- | | | |
| | inducible promoter | | | |
| | (pBBR1 origin, Kan | | | |
| | (R)) | | | |
| P. mirabilis | IdrD:: Tn-Cm(R) | 3237 | 345 | Provided |
| BB2000 IdrD* + | producing IdrD- | | | herein |
| pDS0059 | CT_{E53A} under the | | | |
| | control of an | | | |
| | anhydrotetracyline- | | | |
| | inducible promoter | | | |
| | (pBBR1 origin, Kan | | | |
| | (R)) | | | |
| P. mirabilis | IdrD:: Tn-Cm(R) | 3238 | 346 | Provided |
| BB2000 IdrD* + | producing IdrD- | | | herein |
| pDS0060 | CT_{K55A} under the | | | |
| | control of an | | | |
| | anhydrotetracyline- | | | |
| | inducible promoter | | | |
| | (pBBR1 origin, Kan | | | |
| | (R)) | | | |
| P. mirabilis | IdrD:: Tn-Cm(R) | 3239 | 347 | Provided |
| BB2000 IdrD* + | producing IdrD- | | | herein |
| pDS0061 | CT_{D39A E53A K55A} under | | | |
| | the control of an | | | |
| | anhydrotetracyline- | | | |
| | inducible promoter | | | |
| | (pBBR1 origin, Kan | | | |
| | (R)) | | | |
| E. coli MG1655 + | MG1655 carrying | 2076 | 68 | Provided |
| pBBR1-NheI | empty vector (pBBR1 | | | herein |
| | origin, Kan (R)) | | | |
| E. coli MG1655 + | MG1655 producing | 2298 | 151 | Provided |
| pDS0002 | IdrD-CT under the | | | herein |
| | control of an | | | |
| | anhydrotetracyline- | | | |
| | inducible promoter | | | |
| | (pBBR1 origin, Kan | | | |
| | (R)) | | | |
| E. coli MG1655 + | MG1655 producing | 3228 | 336 | Provided |
| pDS0058 | IdrD-CT_{D39A} under | | | herein |
| | the control of an | | | |
| | anhydrotetracyline- | | | |
| | inducible promoter | | | |
| | (pBBR1 origin, Kan | | | |
| | (R)) | | | |
| E. coli MG1655 + | MG1655 producing | 3229 | 337 | Provided |
| pDS0059 | IdrD-CT_{E53A} under the | | | herein |
| | control of an | | | |
| | anhydrotetracyline- | | | |
| | inducible promoter | | | |
| | (pBBR1 origin, Kan | | | |
| | (R)) | | | |
| E. coli MG1655 + | MG1655 producing | 3230 | 338 | Provided |
| pDS0060 | IdrD-CT_{K55A} under | | | herein |
| | the control of an | | | |
| | anhydrotetracyline- | | | |
| | inducible promoter | | | |
| | (pBBR1 origin, Kan | | | |
| | (R)) | | | |
| E. coli MG1655 + | MG1655 producing | 3231 | 339 | Provided |
| pDS0061 | IdrD-CT_{D39A E53A K55A} | | | herein |
| | under the control of | | | |
| | an anhydrotetracyline- | | | |
| | inducible promoter | | | |
| | (pBBR1 origin, Kan | | | |
| | (R)) | | | |
| OneShot Omnimax | E. coli strain for | | | Thermo |
| 2 T1R Competent | cloning | | | Fisher |
| Cells | | | | Scientific, |
| | | | | Waltham, |
| | | | | MA. |
| S17λpir | E. coli mating strain | 068 | | [52] |
| | to introduce plasmids | | | |
| | into P. mirabilis | | | |

Table 2 shows plasmids used in studies described herein.

| | Cloning method (or | |
|---|---|---|
| Plasmid | source) | Primers and gBlocks (5′=>3′) |
| pBBR1-NheI | | |
| pDS0002 | Anhydrotetracycline | oDS0005 (SEQ ID NO: 17): |
| | promoter (Ptet) with | gctagccatttgcccatgg |
| | 29 bp overhangs | oDS0006 (SEQ ID NO: 18): |
| | (gDS0005) was | cgtttttgataaaaggatattgttgag |
| | recombined into | gDS0005 (SEQ ID NO: 19): |
| | amplified pAS1054 by | tcgcccacccccatgggcaaatggctagcttaagacccactttcacatttaagttgtttttctaat |
| | SLiCE | ccgcatatgatcaattcaaggccgaataagaaggctggctctgcaccttggtgatcaaataat |
| | | tcgatagcttgtcgtaataatggcggcatactatcagtagtaggtgtttccctttcttctttagcga |
| | | cttgatgctcttgatcttccaatacgcaacctaaagtaaaatgccccacagcgctgagtgcata |
| | | taatgcattctctagtgaaaaaccttgttggcataaaaaggctaattgattttcgagagtttcata |
| | | ctgtttttctgtaggccgtgtacctaaatgtacttttgctccatcgcgatgacttagtaaagcacat |
| | | ctaaaacttttagcgttattacgtaaaaaatcttgccagctttccccttctaaagggcaaaagtg |
| | | agtatggtgcctatctaacatctcaatggctaaggcgtcgagcaaagcccgcttattttttacat |
| | | gccaatacaatgtaggctgctctacacctagcttctgggcgagtttacgggttgttaaaccttc |
| | | gattccgacctcattaagcagctctaatgcgctgttaatcactttacttttatctaatctagacatc |
| | | attaattcctaatattgagacactctatcgttgatagagttatataccactccctatcagtgatag |
| | | agaaagtttttgataaaaggatattgttgagcac |
| pDS0058 | The gene encoding | oDS0137(SEQ ID NO: 20): |
| | IdrD-CT_{D39A} was | gtcaaggagctctcatgtgc |
| | amplified from | oDS0159(SEQ ID NO: 21): |
| | pDS0048 (to remove | caataaaccggtctaccattcctcaaacgttatattc |
| | FLAG-tag) and ligated | |
| | in Ptet vector using | |
| | restriction digest (SacI | |
| | and AgeI) | |
| pDS0059 | The gene encoding | Same as above |
| | IdrD-CT_{E53A} was | |
| | amplified from | |
| | pDS0049 (to remove | |
| | FLAG-tag) and ligated | |
| | in Ptet vector using | |
| | restriction digest (SacI | |
| | and AgeI) | |
| pDS0060 | The gene encoding | Same as above |
| | IdrD-CT_{K55A} was | |
| | amplified from | |
| | pDS0050 (to remove | |
| | FLAG-tag) and ligated | |
| | in Ptet vector using | |
| | restriction digest (SacI | |
| | and AgeI) | |
| pDS0061 | The gene encoding | Same as above |
| | IdrD-CT_{D39A E53A K55A} | |
| | was amplified from | |
| | pDS0051 (to remove | |
| | FLAG-tag) and ligated | |
| | in Ptet vector using | |
| | restriction digest (SacI | |
| | and AgeI) | |
| pDS0062 | gfpmut2 was amplified | oDS0161(SEQ ID NO: 22): |
| | and ligated in Ptet | gtacatgagctctcatgagtaaaggagaagaacttttc |
| | vector using restriction | oDS0162 (SEQ ID NO: 23): |
| | digest (SacI and AclI) | caataaaccggtctatttgtatagttcatccatgcc |

### Example 2: P. mirabilis IdrD-CT is a Novel DNase in the PD-(D/E)XK Superfamily

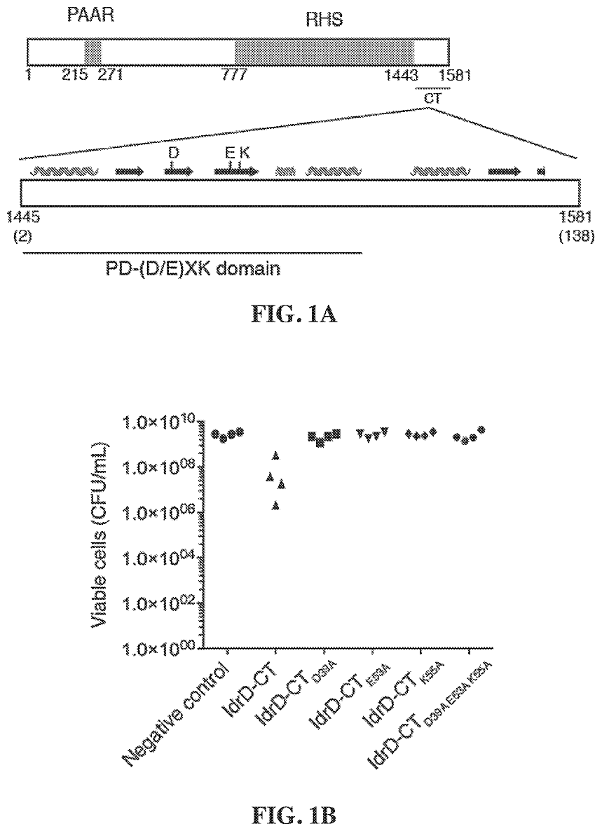

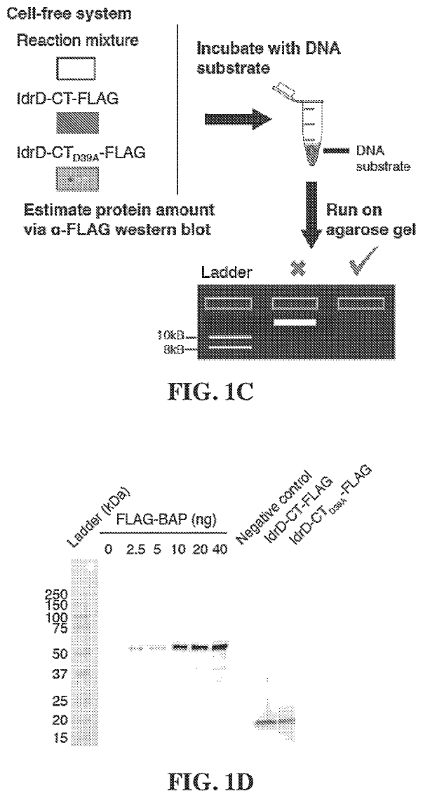

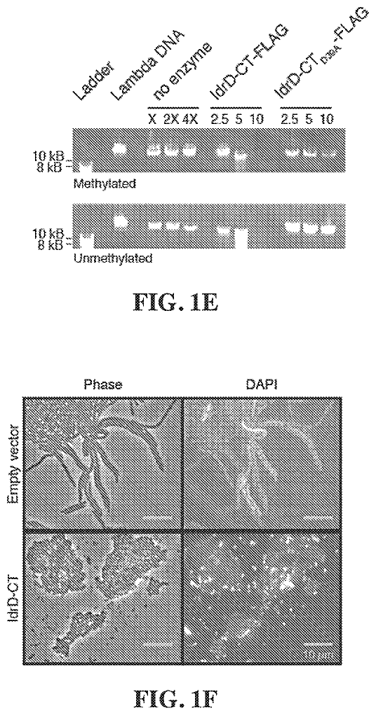

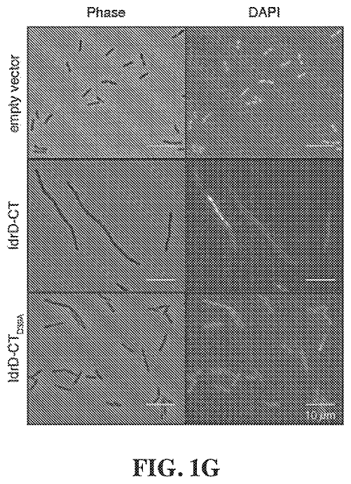

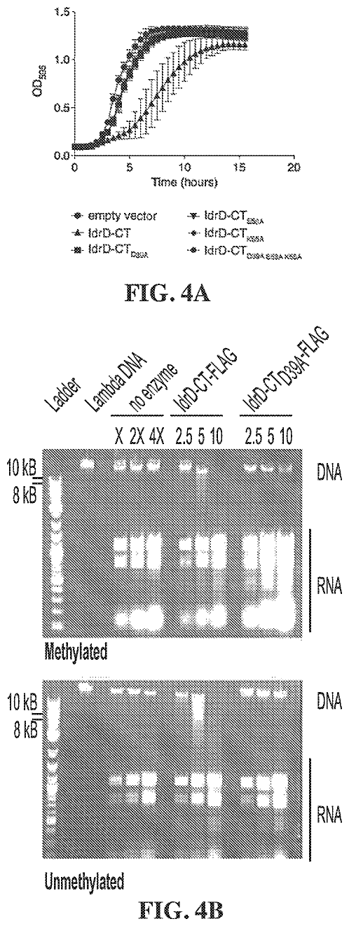

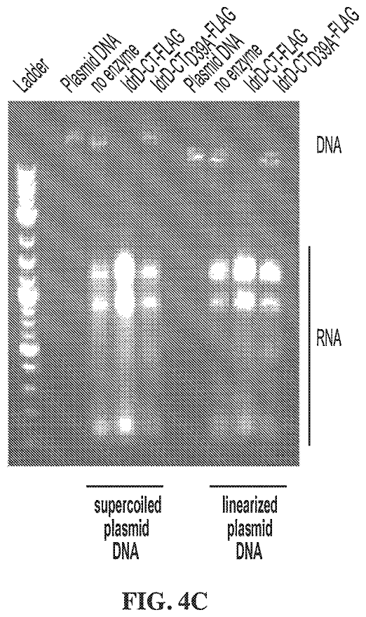

To determine whether IdrD-CT caused death when overproduced in cells, the nucleotide sequence for the predicted IdrD-CT toxin domain was engineered into an anhydrotetracycline-inducible vector. As a negative control, the nucleotide sequence for Green Fluorescent Protein (GFP) was engineered into the parent vector. Vectors were introduced into a P. mirabilis strain in which IdrD and the downstream genes are disrupted [12]. Toxic activity upon protein production was determined by measuring for the number of viable cells after growth on surfaces for 72 hours at room temperature. P. mirabilis producing GFP grew to a saturating density of 10¹⁰, while the strain producing the predicted IdrD-CT toxin grew only to ˜10⁷ (FIG. 1B). An equivalent decrease in viability was observed when IdrD-CT was produced in Escherichia coli (FIG. 4A). Therefore, IdrD-CT causes lethality.

Next, secondary structure predictions of IdrD-CT were examined, and a structural domain found in the PD-(D/E)XK phosphodiesterase superfamily was identified (FIG. 1A). Members of the PD-(D/E)XK phosphodiesterase superfamily include functionally diverse nucleases involved in replication, restriction, DNA repair, and tRNA-intron splicing [24]. The catalytic core and essential residues for nuclease activity (FIG. 1A) are known for this superfamily, having originally been characterized for type II restriction endonucleases [25, 26]. Furthermore, two contact-dependent inhibition (CDI) toxins belong to this superfamily; both are tRNases in Burkholderia pseudomallei (isolates E479 and 1026b) [27]. However, IdrD-CTD itself, as well as similar proteins, comprised a not-yet identified and not-yet characterized subfamily.

To ascertain whether IdrD-CT is a member of the PD-(D/E)XK phosphodiesterase superfamily, mutations were introduced to the essential residues of the predicted catalytic core. The catalytic core was defined based on secondary structure predictions of three beta sheets flanked by two alpha helices as well as the conservation of three residues previously described as critical for the catalytic function of this phosphodiesterase family (FIG. 1A). The three critical residues (D39, E53, and K55) were individually replaced with an alanine residue in the vector-encoded IdrD-CT, and all three mutations residues were disrupted (FIGS. 1A-1B). Viable cells were measured for after 72 hours at room temperature as described herein. All mutant strains displayed increased viability as compared to the wild-type IdrD-CT (FIG. 1B). No lethality was also observed when these mutant proteins were produced in E. coli (FIG. 4A). Therefore, IdrD-CT contains residues consistent with the catalytic core of the PD-(D/E)XK phosphodiesterase superfamily.

To determine the nucleic acid target of IdrD-CT, DNA constructs were engineered to produce either IdrD-CT or IdrD-CT_{D39A}, each of which contained a C-terminal FLAG epitope tag, in a commercial PURExpress cell-free system (FIG. 1C). As described herein, the IdrD-CT_{D39A} protein was less effective at causing cell death the IdrD-CT protein (FIG. 1B). Each protein (˜17 kDa) was produced as confirmed by using electrophoresis across a Tris-tricine gel followed by Western blot analysis with an anti-FLAG antibody (FIG. 1D). Protein yields of IdrD-CT-FLAG were low, suggesting that either DNA, tRNA, and/or rRNA was self-limiting in reaction mixture.

As rRNA and tRNA are already present in the reaction mixture, additional DNA was provided and then assayed for nuclease activity. Samples were analyzed after a 1-hour incubation at 37° C. by electrophoresis on 1% agarose gels stained with ethidium bromide (FIG. 1C). A reaction with no template DNA was used as a negative control in the nuclease activity assays. The ability to cut methylated or unmethylated DNA was examined, and if so, whether degradation was correlated with protein amount. Increasing amounts of PURExpress reaction were added to produce increasing quantities of IdrD-CT-FLAG or IdrD-CT_{D39A}-FLAG, from 2.5 ng to 10 ng. Degradation of both methylated and unmethylated lambda DNA in the presence of IdrD-CT-FLAG was observed (FIG. 1E). The band intensity of lambda DNA inversely correlated with the amount of IdrD-CT protein present (FIG. 1E). By contrast, degradation was not apparent in samples containing the negative control or IdrD-CTD_{39A}-FLAG, even at 10 ng (FIG. 1E and FIG. 4B). Further, bands corresponding to RNA increased in intensity as the amount of PURExpress reaction mixture was increased across samples (FIG. 4B), suggesting that IdrD-CT does not degrade RNA under these conditions.

To examine whether IdrD-CT was capable of endonuclease activity, degradation of supercoiled or linearized plasmid DNA (˜13,500 bp) was measured using equivalent reaction conditions. A single band at the expected size was observed for samples containing the purified DNA, the negative control, or IdrD-CT_{D39A}-FLAG but not for the sample containing IdrD-CT-FLAG (FIG. 4C). Therefore, IdrD-CT is an endonuclease targeting DNA that belongs to the PD-(D/E)XK phosphodiesterase superfamily.

### Example 3: IdrD-CT Subfamily of Proteins Require the Catalytic Core and an Extended C-Terminal Domain for Function

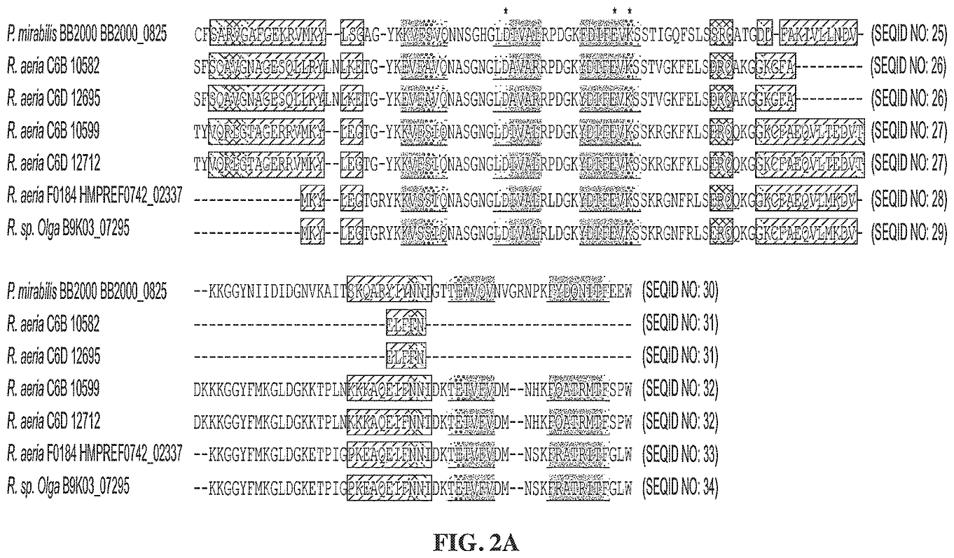

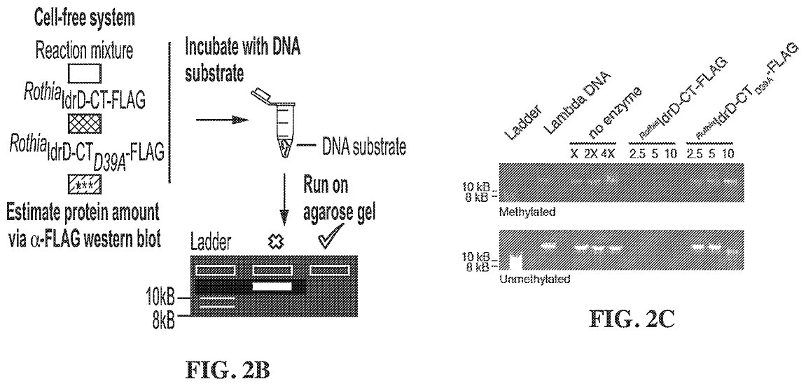

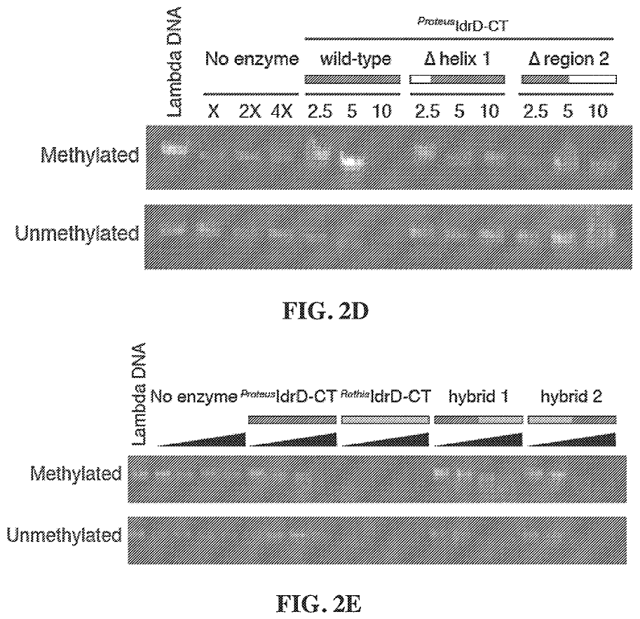

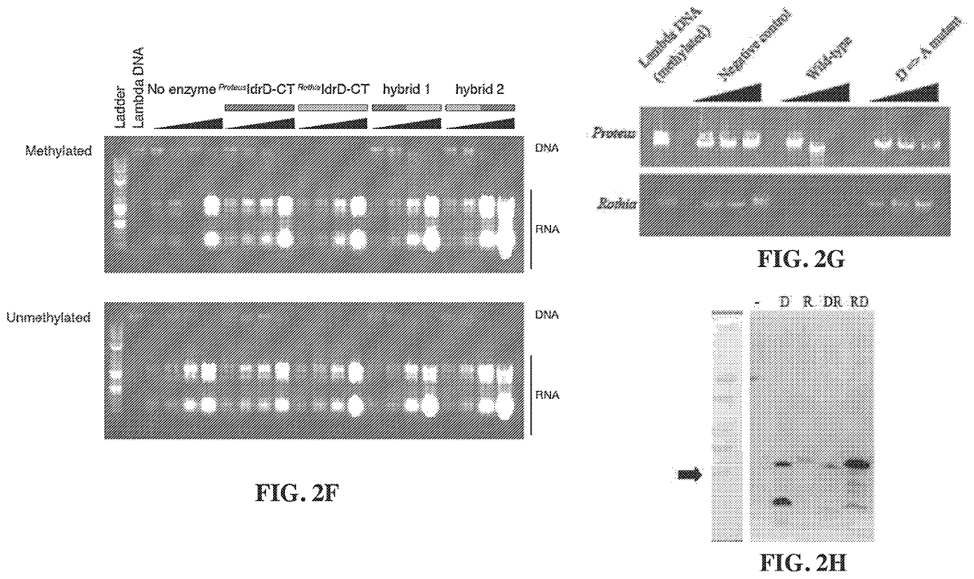

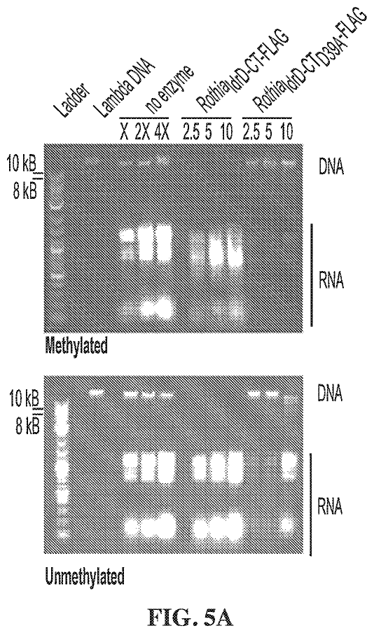

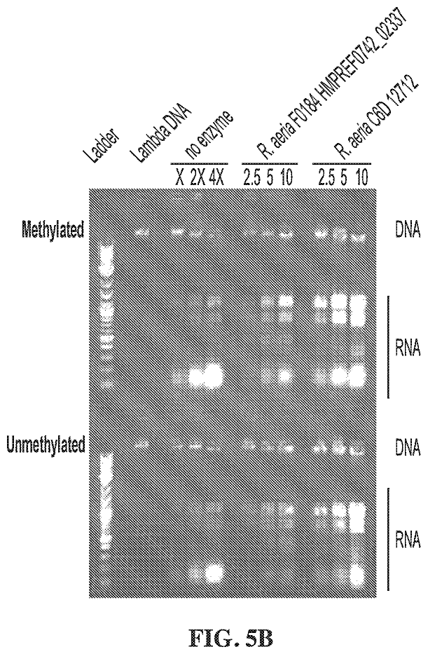

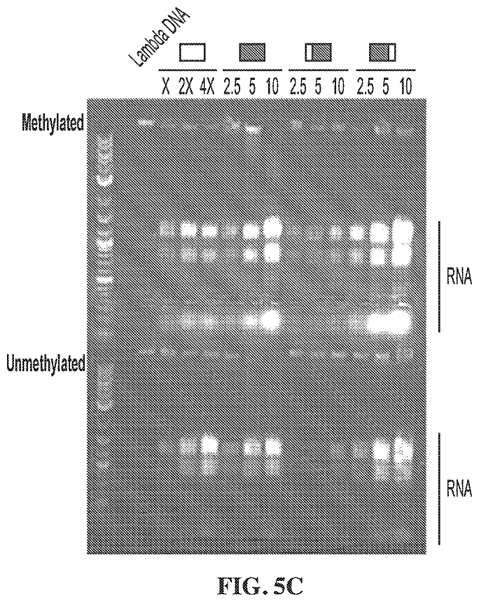

Having found that IdrD-CTD represents a new DNase, this gene was searched for in other bacteria to ascertain its distribution and to define the protein subfamily. By querying public sequence databases, it found that the closest predicted protein with homology to IdrD-CTD is found in Rothia spp., which are Gram-positive inhabitants of the normal flora within the human oral cavity and pharynx [4, 28, 29]. Interactions between P. mirabilis and Rothia have not been reported. The identified proteins were found in R. aeria F0184, R. sp. Olga, R. aeria C6B, and R. aeria C6D with the latter two containing two copies per genome (FIG. 2A). All predicted peptides contained the critical residues of the catalytic core (FIG. 2A). To evaluate whether the R. aeria C6B_10599 protein contained DNA-degrading activity, DNA constructs were engineered to either produce ^{Rothia}IdrD-CT or ^{Rothia}IdrD-CT_{D39A}, each of which contained a C-terminal FLAG epitope tag, in a PURExpress cell-free reaction mixture (FIG. 2B). Like ^{Proteus}IdrD-CT_{D39A}, the ^{Rothia}IdrD-CT_{D39A} construct contains a disruption in the catalytic core. The Rothia-originating peptides were subjected to equivalent analyses as the P. mirabilis-originating proteins (FIG. 2B). Degradation of both methylated and unmethylated lambda DNA was observed in the presence of ^{Rothia}IdrD-CT-FLAG, starting at 2.5 ng of protein (FIG. 2C and FIG. 5A). By contrast, degradation was not apparent in samples containing the negative control or ^{Rothia}IdrD-CT_{D39A}-FLAG (FIG. 2C and FIG. 5A). Bands corresponding to rRNA increased in intensity with increasing volumes of PURExpress reaction (FIG. 5A), suggesting that ^{Rothia}IdrD-CT-FLAG is primarily targeting DNA in these reactions. Therefore, ^{Rothia}IdrD-CT-FLAG is also a DNase.

The sequences originating from Rothia strains differed in length, suggesting that each variant might function differently. The predicted proteins from R. aeria F0184 and R sp. Olga lack part of the N-terminal region where the first alpha helix of the catalytic core resides, whereas C6B_10582 and C6D_12695 encode peptides that lack the C-terminal region after the catalytic core (FIG. 2A and FIG. 5B). Each of these truncated Rothia homologs were produced using the in vitro translation system and assayed for degradation of lambda DNA as described herein. It was found that none of these proteins have DNase activity in spite of containing the three catalytic residues (FIG. 5B). Next, whether either the equivalent N-terminal region or C-terminal region of IdrD-CTD from P. mirabilis, herein referred to as “^{Proteus}IdrD-CT”, was required for DNase activity was examined. Proteins were engineered and produced with individual deletions of each corresponding region in ^{Proteus}IdrD-CT-FLAG and assayed for DNase activity as described here. It was found that deletion of either the N-terminal region or the C-terminal region in ^{Proteus}IdrD-CT resulted in no degradation of methylated and unmethylated lambda DNA (FIG. 2D and FIG. 5C). Thus, the full length of ^{Proteus}IdrD-CT is required for DNA degradation activity.

### Example 4: Metagenome Mapping Reveals Abundance Patterns of IdrD in the Human Microbiome

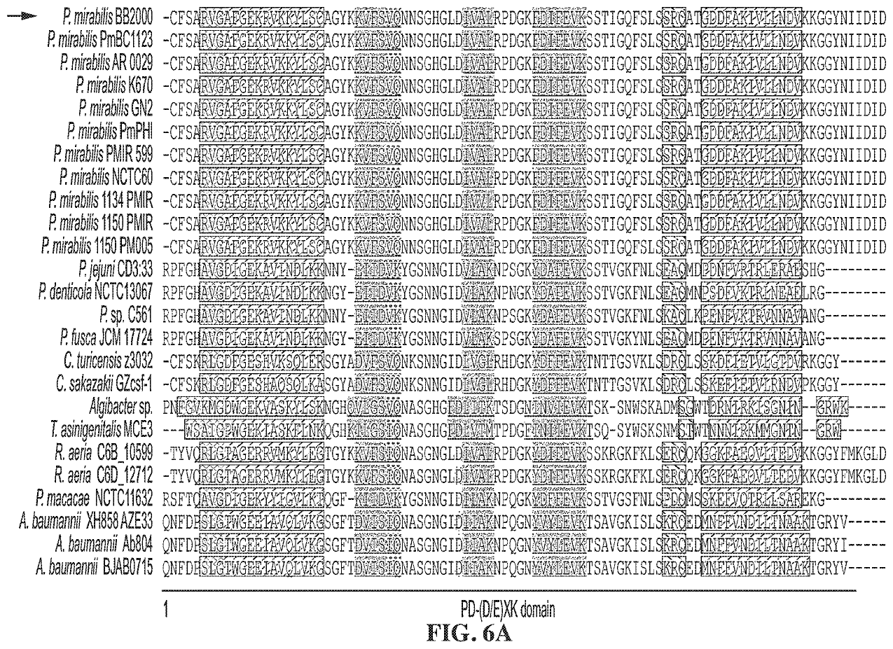

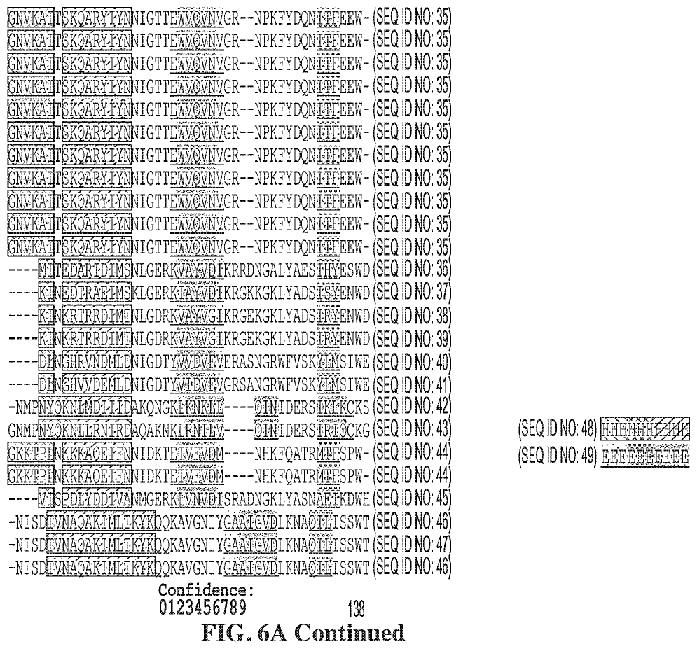

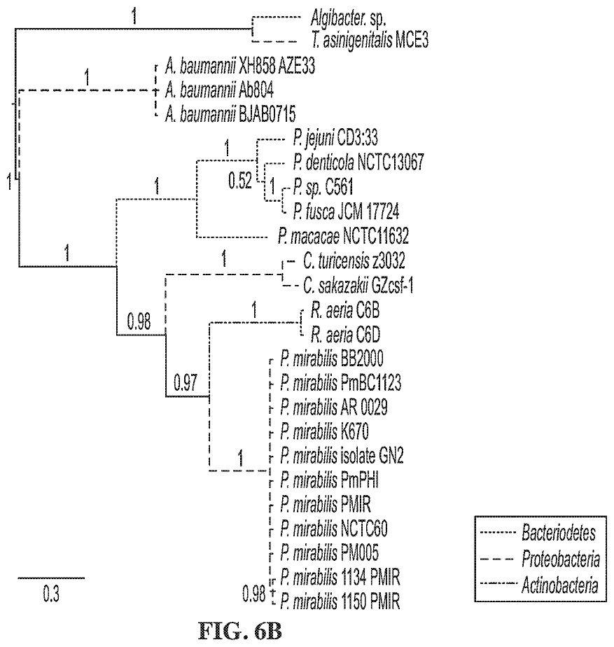

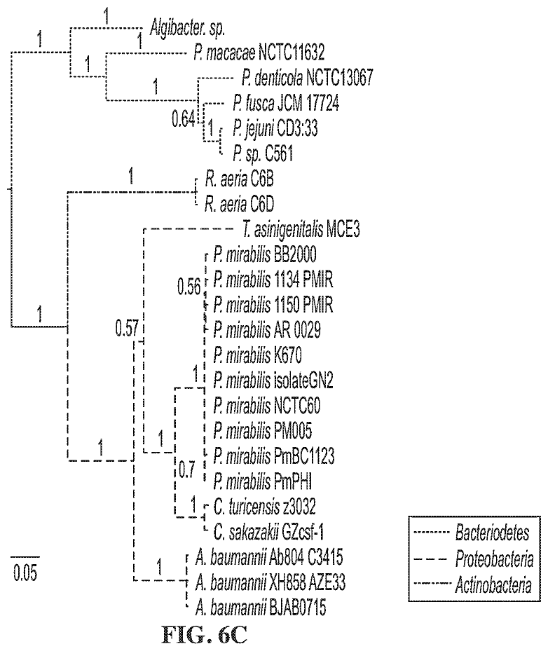

Studies described herein demonstrate that ^{Proteus}IdrD-CT and ^{Rothia}IdrD-CT comprise a previously unknown DNA-targeting subfamily of the PD-(D/E)XK phosphodiesterase superfamily. To identify additional potential members, publicly accessible databases were searched. Employing hmmer [31] and tblastx [32] on the Proteus and Rothia IdrD-CTD sequences, 23 additional proteins that are full-length and conserve the critical catalytic core residues were found (FIGS. 6A and 6B). Phylogenetic reconstruction of the 25 identified proteins showed that these proteins are found across the bacterial tree (FIG. 6B). The predicted secondary structures are consistent across the length of the predicted proteins (FIG. 6A). Protein sequences within a specific group, e.g., genus or species, are more related than to those of different groups (FIG. 6B). However, a species tree based on the full-length 16S rRNA gene (FIG. 6C) did not align with the protein-based tree (FIG. 6B), suggesting that proteins from evolutionarily distant bacteria share more similarity than with more related bacteria. Yet, these bacteria are predominantly human-associated, with many being members of the human gut or oral microbiome and/or are in low abundance.

Given that there is diversity in this protein's sequences between phyla, it was examined whether the IdrD-CT-like nucleotide sequences could be used to resolve the prevalence of this DNA-targeting protein subfamily in human-associated bacterial communities.

Therefore, the representation of IdrD-like nucleotide sequences in the human microbiome as captured by metagenomes was investigated. Publicly-available metagenomes, which are deep, short-read sequencing of random genomic DNA in a sample, were screened with the PARTIE algorithm using full-length IdrD sequences from P. mirabilis BB2000, R. aeria C6B, Cronobacter turicensis z3032, and Prevetolla jejuni CD3:33 (FIG. 4A) [34]. The IdrD-like sequences generally included an extended 5′ region containing a VENN or Rhs motif, and the encoding sequence for the IdrD-CT-like protein was found at the 3′ end [7, 33]. Studies were focused on 319 high-quality metagenomes that were annotated as originating from the human gut or the human oral cavity. Irrelevant metagenomes for each IdrD-like sequence were then filtered out by retaining only metagenomes covering at least half of the IdrD nucleotide sequence. This mapping analysis reports the number of times a metagenomic read mapped to a position along the IdrD-like sequence (FIG. 3A), reflecting the abundance of that specific sequence in a metagenome. Smoothly-decreasing coverage at the terminal 5′ and 3′ ends of a gene remains an artifact of the mapping process. Diversity in IdrD-like sequences, such as deletions or too many nucleotide polymorphisms to be mapped, manifests as changes in coverage along the length of the gene. Ultimately, the coverage of a given region within an IdrD-like sequence reflects the proportional abundance of that region among in a single metagenomic dataset.

While relatively large populations contain IdrD-like sequences, only a relatively small subpopulation appear to harbor a IdrD-CT-like domain. This metagenome mapping revealed that IdrD-like sequences are diverse and present in 45% of the human microbiome samples that we analyzed (FIG. 3B). Each IdrD-like sequence was abundant primarily in metagenomes reported to host its parent organism (e.g., ^{Proteus}IdrD was abundant in gut but not oral microbiomes), suggesting that this mapping analysis could resolve IdrD-like sequences to at least the genus level. Across the entire gene sequence, coverage trends were highly variable, likely reflecting varying degrees of conservation and/or presence among subpopulations. For example, the Rhs core domain generally attracted much more coverage than the rest of the sequence (FIG. 3B, positions marked with dark green bar). This coverage pattern likely represents multiple Rhs-like genes within a single genera given that the Rhs core sequence is relatively conserved. Of note, sufficient diversity likely exists at the nucleotide level even within the Rhs domains. It was found that each individual genus exhibited distinct coverage patterns (FIG. 3B). It was further found that the 3′ region encoding the IdrD-CT-like domains (CTD, light green bar in FIG. 3B) recruited substantially less but more even coverage and was present in just 8.5% of the metagenomes analyzed (FIGS. 3B-3C). These results likely reflect that each IdrD-CT variant is restricted to a narrower subpopulation.

Yet even at this low abundance, the coverage patterns combined with the functional domain analysis of IdrD-CT hinted that there was more complexity to the occurrence of IdrD-CT proteins in the microbiomes. Specifically, the 5′ region of the P. jejuni IdrD-CT recruited reads abundantly from oral microbiomes, where the catalytic core resides, and comparably less in the 3′ region (FIG. 3C). P. jejuni CD3:33 is from a gut isolation, while other Prevotella are often isolated from oral samples. To determine whether this coverage pattern reflected a true truncation, the same oral metagenomes were mapped against IdrD sequences from P. jejuni CD3:33, P. sp. C561, and P. fusca JCM 17724 (FIG. 3D). The entirety of the P. sp. C561 and P. fusca JCM 17724 sequences received even coverage with few SNPs, while the P. jejuni CD3:33 sequence still received little to no coverage across the 3′ region. Thus, the 3′ region of this gene contains species-level differences within the Prevotella genus, raising the possibility that such nucleotide diversity could be used to detect species-level differences in metagenomic datasets.

### Example 5: IdrD-CT is Modular and Contains an Interchangeable Species-Identifying Domain

In the sequence alignments of the identified proteins (FIG. 6A), the C-terminal region contained greater variability in amino acid composition across family members than the N15 terminal region. It was hypothesized that IdrD-CT proteins have two domains: one containing the PD-(D/E)XK motif and one containing species-identifying information. Therefore, independent deletions of each domain in the P. mirabilis IdrD-CT protein were made and assayed for activity. Both deletion proteins resulted in no loss of lambda DNA (FIG. 2D). Equivalent truncations of the Rothia IdrD-CT protein also showed no DNA degradation (FIG. 2E). Therefore, both the enzymatic and species-identifying domains are essential for IdrD-CT activity. The variation in the species-identifying domain hinted that the IdrD-CT protein might be modular. If so, then the species-identifying domain should be exchangeable among variants. To test this assertion, the 3′ region was swapped after the second predicted alpha-helix, between the IdrD-CT proteins of P. mirabilis and R. aeria. The hybrid IdrD-CT proteins degraded lambda DNA while the negative control did not (FIG. 2E). DNA degradation by these hybrid proteins required more protein than the wild-type enzymes, indicating an attenuation in protein activity (FIG. 2E). As the IdrD-CT proteins are flexible in structural design and can tolerate large domain replacements, we reasoned that one could take advantage of the species-identifying sequences to detect distinct proteins in mixed-species communities.

### Example 6: Discussion

Complex microbial communities, such as the gut and oral microbiomes, have historically been defined by the identity of the composite bacteria. Communities, such as those within the oral microbiome, can retain structural organization in which genetically similar bacterial populations are physically separated and inhabit distinct regions [1-4]. However, identifying the processes by which populations maintain niche specificity and spatial separation remains an ongoing area of research. Potential key candidates for defining community structure are contact-dependent toxic proteins [5]. Bacteria can deploy various contact-dependent transport systems to deliver toxins or effector proteins into neighboring cells, often killing the recipient cell or causing long-term growth inhibition [6, 7]. Genetically identical siblings resist death due to the production of a protein to inhibit the delivered toxin [6-8]. The ability to resolve the prevalence of such toxic proteins and to identify functional subdomains remains limited for microbiomes, partially due to the low abundance of these proteins.

The studies described herein have combined molecular and metagenomic analyses of a single protein to address the role of a toxic self-recognition protein in community structure. In the bacterium Proteus mirabilis, which is a low-abundance member of the mammalian gut, one strain is able to physically and spatially exclude another [9]. Many molecular details governing this population separation have been described [10-18]. One critical factor is a previously uncharacterized Rhs toxin, encoded by the gene IdrD (FIG. 1A) [12]. Polymorphic toxin systems, such as the Rhs protein family, are widespread among Gram-negative and Gram-positive bacteria and are characterized by a modular organization [6-8, 19]. These toxins are often exported from the bacterial cell via a diverse set of mechanisms, such as Types IV, V, and VI secretion systems [20-22]. The majority of the protein, near the N-terminus in general, is required for secretion and possible horizontal gene transfer, while the C-terminal region, often ˜140 amino acids in length, harbors toxic activity. These C-terminal domains are found associated with distinct N-terminal regions, suggesting that multiple secretion mechanisms can deliver apparently homologous toxins [7, 8, 19]. Several Rhs toxins have been identified as RNases, DNases, pore-formers, deaminases, and peptidases [23]. However, the function of the IdrD-encoded toxin had yet to be elucidated. Based on amino acid similarity, transport and Rhs subdomains were identified in the predicted IdrD protein, and from that, predicted a potential toxin domain, which was termed “IdrD-CT” (FIG. 1A).

The predicted IdrD-CT protein resembles many polymorphic toxins. In addition to being encoded within a gene with the conserved Rhs repetitive element, this protein is physically transferred from one cell into its adjacent neighbor [12]. This protein is only found in a subset of P. mirabilis strains and is encoded within a gene cluster that varies in gene content between strains [12]. The IdrD-CT protein, however, was classified as having an unknown function. Therefore, studies described herein elucidated the function of IdrD-CT, and the prevalence of this protein and related proteins among human-associated microbial populations.

By combining biochemical characterization and metagenomic analyses of ^{Proteus}IdrD-CT and similar proteins, it was demonstrated that one is able to probe for the abundance of not only the gene as a whole, but also of subdomains within the gene. Here, it was elucidated that ^{Proteus}IdrD-CT and ^{Rothia}IdrD-CT are endonuclease enzymes targeting DNA; activity requires both the catalytic core and a 3′-subdomain.

Further, it was found that the varying 3′ region of the IdrD nucleotide sequence is a candidate region to define sub-population identity and could potentially allow for detection of species-level differences in metagenomic datasets. The 5′ region contains the catalytic core and may be protected from mutations due to constraints in the three-dimensional space for enzymatic function [26, 36-40]. The 3′-region of ^{Proteus}IdrD-CT is essential and has more variation between amino acid sequences across phyla. Therefore, the 3′-region appears more flexible in sequence while still retaining function. If so, it might be more accessible for accumulating mutations allowing for each IdrD-CT to become distinct.

IdrD-CT, a P. mirabilis self-recognition protein that acts on adjacent cells through direct contact [12] is also described herein. IdrD-CT is the founding member for a family of DNases; these proteins are modular and contain an interchangeable species-identifying domain. This has been termed a family of “Idr-PDE-DNases” herein. Through a combination of genetics and molecular biology along with biochemical and phylogenetic interrogation, it has been shown that IdrD-CT from P. mirabilis and R. aeria degrade DNA regardless of methylation state (FIGS. 1B, 1E, and 6A). Idr-PDE-DNase proteins are found in evolutionarily distant bacteria and together form a distinct family of the PD-(D/E)XK nuclease superfamily. Finding and characterizing additional families is crucial for optimizing methods to predict function from sequence for this superfamily.

Further, it was demonstrated that metagenomic analysis can provide fine functional resolution of the Idr-PDE-DNase proteins in human samples, even when sequences are present at low frequency in datasets. For example, the 3′ region of the IdrD-CT nucleotide sequence distinguished gut and oral Prevotella species (FIG. 3D), reflecting diversity in bacterial populations naturally occurring in human guts and oral cavities. Flexibility in sequence space for this 3′ region might allow each Idr-PDE-DNase protein to become distinct among recently diverged groups such as species.

How gene clusters encoding strain-specific proteins (e.g., toxins or effectors) evolve is an intriguing question in multiple contexts. Two-pair toxic protein systems are analogous to colonization and addiction proteins or mating compatibility such as in Wolbachia [58] and in Caenorhabditis elegans [59]. One current hypothesis is that speciation occurs on the gene cluster of the effector protein (toxin) and its immunity protein (anti-toxin), for example, through horizontal gene transfer, as these two-gene pairs are often in variable hotspots on the genome [6-8, 19]. Yet for these Idr-PDE-DNase proteins, a pair of domains within a protein follow such a tandem pattern, raising the possibility that an evolutionary arms race, internal to the protein, may also drive speciation. Frequent horizontal gene transfer and/or shifting selection pressures could also explain the sequence variability within the Idr-PDE-DNase proteins.

Of the two essential domains of the Idr-PDE-DNase proteins, one has enzyme activity and is conserved, while the other is variable and contains information specific to genus and species. A need to maintain enzymatic activity [26, 36, 37, 39, 40, 60] might restrict the conserved enzymatic domain from changing. By contrast, the C-terminal half of Idr-PDE-DNase proteins vary in amino acid sequences. Similar DNA-targeting proteins act as multimeric complexes, and similar effector proteins can bind allele-specific immunity proteins [6-8, 35]. The C-terminus of Idr-PDE-DNase proteins might contribute to protein-binding and/or DNA protein interactions, especially as the predicted secondary structures are similar among these proteins. The functional exchange of the C-terminal region between two Idr-PDE-DNase (from Proteus and Rothia) proteins are consistent with this hypothesis (FIG. 2E).

Unlike chimeric nucleases such as homing nucleases, zinc finger nucleases, TALENS, and CRISPR/Cas9 (reviewed in [61, 62]), the DNA recognition motif for the Idr-PDE-DNase proteins is unknown. Moreover, the non-nuclease domain does not seem to contain a typical DNA-binding motif. Structural studies are needed to discern how Idr-PDE-DNase proteins differ in interactions with DNA and protein partners. Further, capitalizing on fine-detail differences as species-identifying markers allows one to pull out rare species, and perhaps strains, in naturally occurring communities. For example, the sequence differences in the variable region of IdrD-CT acted as tokens for identifying distinct strains or species in metagenomic datasets. Molecular identification of sequence variants with known functional consequences also informs physiologically relevant population boundaries when resolved in large sequence datasets. Combining metagenomic analysis with molecular characterization of strain-identifying factors opens a window into understanding how interactions among resident microbes might contribute to behaviors within a host.

### REFERENCES

1. Lloyd-Price, J., et al., Strains, functions and dynamics in the expanded Human Microbiome Project. Nature, 2017. 550(7674): p. 61-66.
2. Costea, P. I., et al., Subspecies in the global human gut microbiome. Mol Syst Biol, 2017. 13(12): p. 960.
3. Eren, A. M., et al., Oligotyping analysis of the human oral microbiome. Proc Natl Acad Sci USA, 2014. 111(28): p. E2875-84.
4. Aas, J. A., et al., Defining the normal bacterial flora of the oral cavity. J Clin Microbiol, 2005. 43(11): p. 5721-32.
5. Verster, A. J., et al., The Landscape of Type VI Secretion across Human Gut Microbiomes Reveals Its Role in Community Composition. Cell Host Microbe, 2017. 22(3): p. 411-419.e4.
6. Koskiniemi, S., et al., Rhs proteins from diverse bacteria mediate intercellular competition. Proc Natl Acad Sci USA, 2013. 110(17): p. 7032-7.
7. Aoki, S. K., et al., A widespread family of polymorphic contact-dependent toxin delivery systems in bacteria. Nature, 2010. 468(7322): p. 439-42.
8. Zhang, D., L. M. Iyer, and L. Aravind, A novel immunity system for bacterial nucleic acid degrading toxins and its recruitment in various eukaryotic and DNA viral systems. Nucleic Acids Res, 2011. 39(11): p. 4532-52.
9. Dienes, L., Reproductive processes in Proteus cultures. Proc Soc Exp Biol Med, 1946. 63(2): p. 265-70.
10. Gibbs, K. A., M. L. Urbanowski, and E. P. Greenberg, Genetic determinants of self identity and social recognition in bacteria. Science, 2008. 321(5886): p. 256-9.
11. Gibbs, K. A., L. M. Wenren, and E. P. Greenberg, Identity gene expression in Proteus mirabilis. J Bacteriol, 2011. 193(13): p. 3286-92.
12. Wenren, L. M., et al., Two independent pathways for self-recognition in Proteus mirabilis are linked by type VI-dependent export. MBio, 2013. 4(4).
13. Alteri, C. J., et al., Multicellular bacteria deploy the type VI secretion system to preemptively strike neighboring cells. PLoS Pathog, 2013. 9(9): p. e1003608.
14. Cardarelli, L., C. Saak, and K. A. Gibbs, Two Proteins Form a Heteromeric Bacterial Self-Recognition Complex in Which Variable Subdomains Determine Allele-Restricted Binding. MBio, 2015. 6(3): p. e00251.
15. Saak, C. C. and K. A. Gibbs, The Self-Identity Protein IdsD Is Communicated between Cells in Swarming Proteus mirabilis Colonies. J Bacteriol, 2016. 198(24): p. 3278-3286.
16. Alteri, C. J., et al., Subtle variation within conserved effector operon gene products contributes to T6SS-mediated killing and immunity. PLoS Pathog, 2017. 13(11): p. e1006729.
17. Zepeda-Rivera, M. A., C. C. Saak, and K. A. Gibbs, A Proposed Chaperone of the Bacterial Type VI Secretion System Functions To Constrain a Self-Identity Protein. J Bacteriol, 2018. 200(14).
18. Tipping, M. J. and K. A. Gibbs, Peer pressure from a Proteus mirabilis self-recognition system controls participation in cooperative swarm motility. bioRxiv 490771, 2018.
19. Jackson, A. P., et al., Evolutionary diversification of an ancient gene family (rhs) through C-terminal displacement. BMC Genomics, 2009. 10: p. 584.
20. Alvarez-Martinez, C. E. and P. J. Christie, Biological diversity of prokaryotic type IV secretion systems. Microbiol Mol Biol Rev, 2009. 73(4): p. 775-808.
21. Aoki, S. K., et al., Contact-dependent inhibition of growth in Escherichia coli. Science, 2005. 309(5738): p. 1245-8.
22. Russell, A. B., et al., Type VI secretion delivers bacteriolytic effectors to target cells. Nature, 2011. 475(7356): p. 343-7.
23. Zhang, D., et al., Polymorphic toxin systems: Comprehensive characterization of trafficking modes, processing, mechanisms of action, immunity and ecology using comparative genomics. Biol Direct, 2012. 7: p. 18.
24. Steczkiewicz, K., et al., Sequence, structure and functional diversity of PD-(D/E)XK phosphodiesterase superfamily. Nucleic Acids Res, 2012. 40(15): p. 7016-45.
25. Selent, U., et al., A site-directed mutagenesis study to identify amino acid residues involved in the catalytic function of the restriction endonuclease EcoRV. Biochemistry, 1992. 31(20): p. 4808-15.
26. Venclovas, C., A. Timinskas, and V. Siksnys, Five-stranded beta-sheet sandwiched with two alpha-helices: a structural link between restriction endonucleases EcoRI and EcoRV. Proteins, 1994. 20(3): p. 279-82.
27. Johnson, P. M., et al., Functional Diversity of Cytotoxic tRNase/Immunity Protein Complexes from Burkholderia pseudomallei. J Biol Chem, 2016. 291(37): p. 19387-400.
28. ROTH, G. D. and A. N. THURN, Continued study of oral nocardia. J Dent Res, 1962. 41:p. 1279-92.
29. Georg, L. K. and J. M. Brown, <i style=“ ”>Rothia, gen. nov. an aerobic genus of the family Actinomycetaceae. International Journal of Systematic and Evolutionary Microbiology, 1967. 17: p. 79-88.
30. Zimmermann, L., et al., A Completely Reimplemented MPI Bioinformatics Toolkit with a New HHpred Server at its Core. J Mol Biol, 2018. 430(15): p. 2237-2243.
31. Eddy, S. R., A new generation of homology search tools based on probabilistic inference. Genome Inform, 2009. 23(1): p. 205-11.
32. Altschul, S. F., et al., Basic local alignment search tool. J Mol Biol, 1990. 215(3): p. 403-10.
33. Lin, R. J., M. Capage, and C. W. Hill, A repetitive DNA sequence, rhs, responsible for duplications within the Escherichia coli K-12 chromosome. J Mol Biol, 1984. 177(1): p. 1-18.
34. Torres, P. J., R. A. Edwards, and K. A. McNair, PARTIE: a partition engine to separate metagenomic and amplicon projects in the Sequence Read Archive. Bioinformatics, 2017. 33(15): p. 2389-2391.
35. Kosinski, J., M. Feder, and J. M. Bujnicki, The PD-(D/E)XK superfamily revisited: identification of new members among proteins involved in DNA metabolism and functional predictions for domains of (hitherto) unknown function. BMC Bioinformatics, 2005. 6: p. 172.
36. Skirgaila, R., et al., Structure-based redesign of the catalytic/metal binding site of Cfr10I restriction endonuclease reveals importance of spatial rather than sequence conservation of active centre residues. J Mol Biol, 1998. 279(2): p. 473-81.
37. Bujnicki, J. M. and L. Rychlewski, Identification of a PD-(D/E)XK-like domain with a novel configuration of the endonuclease active site in the methyl-directed restriction enzyme Mrr and its homologs. Gene, 2001. 267(2): p. 183-91.
38. Pingoud, V., et al., Evolutionary relationship between different subgroups of restriction endonucleases. J Biol Chem, 2002. 277(16): p. 14306-14.
39. Tamulaitis, G., A. S. Solonin, and V. Siksnys, Alternative arrangements of catalytic residues at the active sites of restriction enzymes. FEBS Lett, 2002. 518(1-3): p. 17-22.
40. Feder, M. and J. M. Bujnicki, Identification of a new family of putative PD-(D/E)XK nucleases with unusual phylogenomic distribution and a new type of the active site. BMC Genomics, 2005. 6: p. 21.
41. Mark Welch, J. L., et al., Biogeography of a human oral microbiome at the micron scale. Proc Natl Acad Sci USA, 2016. 113(6): p. E791-800.
42. Egan, F., F. J. Reen, and F. O'Gara, The distribution and diversity in metagenomic datasets reveal niche specialization. Environ Microbiol Rep, 2015. 7(2): p. 194-203.
43. Edgar, R. C., MUSCLE: multiple sequence alignment with high accuracy and high throughput. Nucleic Acids Res, 2004. 32(5): p. 1792-7.
44. Capella-Gutiérrez, S., J. M. Silla-Martinez, and T. Gabaldón, trimAl: a tool for automated alignment trimming in large-scale phylogenetic analyses. Bioinformatics, 2009. 25(15): p. 1972-3.
45. Ronquist, F. and J. P. Huelsenbeck, MrBayes 3: Bayesian phylogenetic inference under mixed models. Bioinformatics, 2003. 19(12): p. 1572-4.
46. Whelan, S. and N. Goldman, A general empirical model of protein evolution derived from multiple protein families using a maximum-likelihood approach. Mol Biol Evol, 2001. 18(5): p. 691-9.
47. S, T., Some probabilistic and statistical problems in the analysis of DNA sequences, in Some Mathematical Questions in Biology. DNA Sequence Analysis, 1986: p. 57-86.
48. Stamatakis, A., RAxML version 8: a tool for phylogenetic analysis and post-analysis of large phylogenies. Bioinformatics, 2014. 30(9): p. 1312-3.
49. Langmead, B. and S. L. Salzberg, Fast gapped-read alignment with Bowtie 2. Nat Methods, 2012. 9(4): p. 357-9.
50. Eren, A. M., et al., Anvi'o: an advanced analysis and visualization platform for ‘omics data. PeerJ, 2015. 3: p. e1319.
51. Shannon, C. E., A mathematical theory of communication. The Bell System Technical Journal, 1948. 27(3): p. 379-423.
52. Simon, R., U. Priefer, and A. Pühler, A Broad Host Range Mobilization System for In Vivo Genetic Engineering: Transposon Mutagenesis in Gram Negative Bacteria. Nat Biotechnol 1, 784-791 (1983). doi.org/10.1038/nbt1183-784.
53. C. N. Vassallo, D. Wall, Self-identity barcodes encoded by six expansive polymorphic toxin families discriminate kin in myxobacteria. Proc. Natl. Acad. Sci. U.S.A (2019), 15
54. K. S. Makarova, Y. I. Wolf, S. Karamycheva, D. Zhang, L. Aravind, E. V. Koonin, Antimicrobial peptides, polymorphic toxins, and self-nonself recognition systems in Archaea: an untapped armory for intermicrobial conflicts. mBio. 10 (2019)
55. C. E. Armbruster, V. Forsyth-DeOrnellas, A. O. Johnson, S. N. Smith, L. Zhao, W. Wu, H. L. T. Mobley, Genome-wide transposon mutagenesis of Proteus mirabilis: Essential genes, fitness factors for catheter-associated urinary tract infection, and the impact of polymicrobial infection on fitness requirements. PLoS Pathog. 13, e1006434 (2017).
56. D. A. Vlazny, C. W. Hill, A stationary-phase-dependent viability block governed by two different polypeptides from the RhsA genetic element of Escherichia coli K-12. J Bacteriol. 177, 2209-13 (1995).
57. B. D. Ross, A. J. Verster, M. C. Radey, D. T. Schmidtke, C. E. Pope, L. R. Hoffman, A. M. 15 Hajjar, S. B. Peterson, E. Borenstein, J. D. Mougous, Human gut bacteria contain acquired interbacterial defence systems. Nature. 575, 224-228 (2019).
58. H. Chen, J. A. Ronau, J. F. Beckmann, M. Hochstrasser, A Wolbachia nuclease and its binding partner provide a distinct mechanism for cytoplasmic incompatibility. Proc. Natl. Acad. Sci. U.S.A 116, 22314-22321 (2019).
59. E. Ben-David, A. Burga, L. Kruglyak, A maternal-effect selfish genetic element in Caenorhabditis elegans. Science. 356, 1051-1055 (2017).
60. A. Pingoud, A. Jeltsch, Structure and function of type II restriction endonucleases. Nucleic Acids Res. 29, 3705-27 (2001).
61. T. Gaj, C. A. Gersbach, C. F. Barbas, ZFN, TALEN, and CRISPR/Cas-based methods for genome engineering. Trends Biotechnol. 31, 397-405 (2013).
62. D. Carroll, Genome Engineering With Zinc-Finger Nucleases. Genetics. 188, 773-782 (2011).

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. An IdrD protein comprising a fragment of IdrD protein from Proteus mirabilis, a fragment of IdrD protein from Rothia, or a fusion thereof.

2. The IdrD protein of claim 1, wherein the fragment of IdrD protein from Proteus mirabilis comprises an amino acid sequence that is at least 85% identical to the amino acid sequence provided by SEQ ID NO: 1.

3. The IdrD protein of claim 1, wherein the fragment of IdrD protein from Proteus mirabilis comprises an amino acid sequence that is at least 95% identical to the amino acid sequence provided by SEQ ID NO: 1.

4. The IdrD protein of claim 1, wherein the fragment of IdrD protein from Proteus mirabilis comprises an amino acid sequence that is identical to the amino acid sequence provided by SEQ ID NO: 1.

5. The IdrD protein of claim 1, wherein the fragment of IdrD protein from Proteus mirabilis comprises an amino acid sequence having at least one mutation in the amino acid sequence provided by SEQ ID NO: 1.

6. The IdrD protein of claim 5, wherein the at least one mutation is a mutation at amino acid residue 39 of the amino acid sequence provided by SEQ ID NO: 1.

7. The IdrD protein of claim 6, wherein the mutation at amino acid residue 39 is a substitution of aspartic acid with alanine.

8. The IdrD protein of claim 1, wherein the fragment of IdrD protein from Rothia comprises an amino acid sequence that is at least 85% identical to the amino acid sequence provided by SEQ ID NO: 5.

9. The IdrD protein of claim 1, wherein the fragment of IdrD protein from Rothia comprises an amino acid sequence that is at least 95% identical to the amino acid sequence provided by SEQ ID NO: 5.

10. The IdrD protein of claim 1, wherein the fragment of IdrD protein from Rothia comprises an amino acid sequence that is identical to the amino acid sequence provided by SEQ ID NO: 5.

11. The IdrD protein of claim 1, wherein the fragment of IdrD protein from Rothia comprises an amino acid sequence having at least one mutation to the amino acid sequence provided by SEQ ID NO: 5.

12. The IdrD protein of claim 11, wherein the at least one mutation is a mutation at amino acid residue 25 of the amino acid sequence provided by SEQ ID NO: 5.

13. The IdrD protein of claim 12, wherein the mutation at amino acid residue 25 is a substitution of aspartic acid with alanine.

14. The IdrD protein of claim 1, wherein the fusion comprises a fragment of IdrD protein from Proteus mirabilis fused to a fragment of IdrD protein from Rothia.

15. The IdrD protein of claim 14, wherein the fusion thereof comprises an amino acid sequence that is at least 95% identical to the amino acid sequence provided by SEQ ID NO: 9.

16. The IdrD protein of claim 14, wherein the fusion thereof comprises an amino acid sequence that is identical to the amino acid sequence provided by SEQ ID NO: 9.

17. The IdrD protein of claim 1, wherein the fusion thereof comprises the fragment of IdrD protein from Rothia fused to the fragment of IdrD protein from Proteus mirabilis.

18. The IdrD protein of claim 17, wherein the fusion thereof comprises an amino acid sequence that is at least 95% identical to the amino acid sequence provided by SEQ ID NO: 11.

19. The IdrD protein of claim 17, wherein the fusion thereof comprises an amino acid sequence that is identical to the amino acid sequence provided by SEQ ID NO: 11.

20. An expression vector comprising a nucleic acid encoding the IdrD protein of any one of claims 1-19.

21. The expression vector of claim 20, wherein the nucleic acid encoding the IdrD protein is selected from the group consisting of SEQ ID NO: 2, SEQ ID NO: 4, SEQ ID NO: 6, SEQ ID NO: 8, SEQ ID NO: 10, SEQ ID NO: 12, and combinations thereof.

22. A cell comprising the expression vector of claim 20 or 21.

23. A pharmaceutical composition comprising the IdrD protein of any one of claims 1-19 and a pharmaceutically acceptable carrier.

24. The pharmaceutical composition of claim 23 further comprising a therapeutic agent.

25. The pharmaceutical composition of claim 24, wherein the therapeutic agent is an antibiotic.

26. The pharmaceutical composition of claim 24, wherein the therapeutic agent is a chemotherapeutic agent.

27. A method for inhibiting bacterial biofilm formation on a surface, the method comprising contacting or coating the surface with IdrD protein of any one of claims 1-19.

28. The method of claim 27, wherein contacting or coating comprises spraying, brushing, applying, and/or treating the surface.

29. The method of claim 27, wherein the surface is a tissue.

30. The method of claim 29, wherein the tissue is an infected tissue or a wounded tissue.

31. The method of claim 29, wherein the tissue is selected from the group consisting of skin tissue, tumor tissue, and organ tissue.

32. A method for treating a disease associated with bacteria trapping neutrophil extracellular traps (NETs), the method comprising administering to a subject in need thereof a therapeutically effective amount of IdrD protein of any one of claims 1-19.

33. The method of claim 32, wherein the subject is a human.

34. The method of claim 32, wherein the disease associated with bacteria trapping NETs is selected from the group consisting of a wound, an infection, a lung disease, a cancer, and a vascular disease.

35. The method of claim 32, wherein the lung disease is cystic fibrosis.

36. The method of claim 32, wherein the vascular disease is thrombosis.

37. The method of claim 32 further comprising administering a therapeutic agent.

38. The method of claim 37, wherein the therapeutic agent is an antibiotic.

39. The method of claim 37, wherein the therapeutic agent is a chemotherapeutic agent.

40. A method of treating or preventing a bacterial infection, the method comprising administering to a subject in need thereof a therapeutically effective amount of IdrD protein of any one of claims 1-19.

41. The method of claim 40, wherein the bacterial infection is a Proteus mirabilis infection.

42. The method of claim 40 further comprising administering a therapeutic agent.

43. The method of claim 42, wherein the therapeutic agent is an antibiotic.

44. A method for cleaving DNA, the method comprising incubating DNA and an effective amount of IdrD protein of any one of claims 1-19.

45. The method of claim 44, wherein the DNA is obtained from a cell lysate or a cell culture.

46. The method of claim 44, wherein the DNA is obtained from an in vitro reaction mixture.

47. The method of claim 44, wherein the DNA is obtained from a patient.

48. A kit comprising, the IdrD protein of any one of claims 1-19, the expression vector of any one of claims 20-21, the cell of claim 22, or pharmaceutical composition 23-26.

49. The kit of claim 48, further comprising a container.

50. The kit of any one of claims 48-49, further comprising instructions for carrying out use of the kit.

51. The kit of any one of claims 48-50, further comprising instructions for carrying out any of the methods of claims 27-47.


SEQUENCE LISTING (49 sequences)

SEQ ID NO: 1 - protein, 138 aa, Proteus Mirabilis
MCFSARVGAFGEKRVMKYLSGAGYKKVFSVQNNSGHGLDIVALRPDGKFDIFEVKSSTIGQFSLSSRQATGDDFAKIVLLNDVKKGGYNIIDIDGNVKAITSKQARYIYNNIGTTEWVQVNVGRNPKFYDQNITFEEW

SEQ ID NO: 2 - DNA, 417 bp, Proteus Mirabilis
atgtgctttagtgctcgggtaggtgcttttggtgagaaaagagttatgaaatacttatctggagcgggctataaaaaagttttttctgtacaaaacaattctgggcatggtctggatatagttgctttaagaccagatggaaaatttgatatttttgaagttaaaagttcgacaataggacaattttctttatcttcccgccaagctacaggcgatgattttgcaaaaatagttcttttaaacgatgtgaaaaaaggaggttataatattatcgatatagatggtaatgttaaagcaattacaagtaaacaagctagatacatttataataacataggaacaaccgagtgggttcaggtaaatgttggccgaaatcccaaattttatgatcagaatataacgtttgaggaatggtag

SEQ ID NO: 3 - protein, 138 aa, Artificial Sequence
  Synthetic
MCFSARVGAFGEKRVMKYLSGAGYKKVFSVQNNSGHGLAIVALRPDGKFDIFEVKSSTIGQFSLSSRQATGDDFAKIVLLNDVKKGGYNIIDIDGNVKAITSKQARYIYNNIGTTEWVQVNVGRNPKFYDQNITFEEW

SEQ ID NO: 4 - DNA, 417 bp, Artificial Sequence
  Synthetic
atgtgctttagtgctcgggtaggtgcttttggtgagaaaagagttatgaaatacttatctggagcgggctataaaaaagttttttctgtacaaaacaattctgggcatggtctggctatagttgctttaagaccagatggaaaatttgatatttttgaagttaaaagttcgacaataggacaattttctttatcttcccgccaagctacaggcgatgattttgcaaaaatagttcttttaaacgatgtgaaaaaaggaggttataatattatcgatatagatggtaatgttaaagcaattacaagtaaacaagctagatacatttataataacataggaacaaccgagtgggttcaggtaaatgttggccgaaatcccaaattttatgatcagaatataacgtttgaggaatggtag

SEQ ID NO: 5 - protein, 123 aa, Rothia
MKYLEGTGRYKKVSSIQNASGNGLDIVALRLDGKYDIFEVKSSKRGNFRLSERQQKGGKCFAEQVLMKDVKKGGYFMKGLDGKETPIGPKEAQEIFNNIDKTETVFVDMNSKFRATRITFGLW

SEQ ID NO: 6 - DNA, 423 bp, Rothia
atgacctacgtgcagcgcttgggtactgccggcgagcgcagggtaatgaaatatctggaaggtacgggatacaaaaaagtcttctctattcaaaatgcctccggaaacggcttggatattgtcgcattgagaccggatgggaaatatgacatatttgaagttaaaagttctaaacgtggaaaatttaaactaagcgaaagacagcagaaaggcggtaaatgttttgctgagcaagtgttgacggaagatgtaacggataagaaaaaaggcggatattttatgaaaggcttagacggcaaaaagacgcctcttaataagaagaaggcgcaagagatatttaataatatagataaaaccgaaactgtctttgttgatatgaatcataaatttcaagcaacccgcatgacatttagcccatggtaa

SEQ ID NO: 7 - protein, 123 aa, Artificial Sequence
  Synthetic
MKYLEGTGRYKKVSSIQNASGNGLAIVALRLDGKYDIFEVKSSKRGNFRLSERQQKGGKCFAEQVLMKDVKKGGYFMKGLDGKETPIGPKEAQEIFNNIDKTETVFVDMNSKFRATRITFGLW

SEQ ID NO: 8 - DNA, 423 bp, Artificial Sequence
  Synthetic
atgacctacgtgcagcgcttgggtactgccggcgagcgcagggtaatgaaatatctggaaggtacgggatacaaaaaagtcttctctattcaaaatgcctccggaaacggcttggctattgtcgcattgagaccggatgggaaatatgacatatttgaagttaaaagttctaaacgtggaaaatttaaactaagcgaaagacagcagaaaggcggtaaatgttttgctgagcaagtgttgacggaagatgtaacggataagaaaaaaggcggatattttatgaaaggcttagacggcaaaaagacgcctcttaataagaagaaggcgcaagagatatttaataatatagataaaaccgaaactgtctttgttgatatgaatcataaatttcaagcaacccgcatgacatttagcccatggtaa

SEQ ID NO: 9 - protein, 138 aa, Artificial Sequence
  Synthetic
MCFSARVGAFGEKRVMKYLSGAGYKKVFSVQNNSGHGLDIVALRPDGKFDIFEVKSSTIGQFSLSSRQATGDDFAKIVLLNDVKKKKGGYFMKGLDGKKTPLNKKKAQEIFNNIDKTETVFVDMNHKFQATRMTFSPW

SEQ ID NO: 10 - DNA, 417 bp, Artificial Sequence
  Synthetic
atgtgctttagtgctcgggtaggtgcttttggtgagaaaagagttatgaaatacttatctggagcgggctataaaaaagttttttctgtacaaaacaattctgggcatggtctggatatagttgctttaagaccagatggaaaatttgatatttttgaagttaaaagttcgacaataggacaattttctttatcttcccgccaagctacaggcgatgattttgcaaaaatagttcttttaaacgatgtgaaaaagaaaaaaggcggatattttatgaaaggcttagacggcaaaaagacgcctcttaataagaagaaggcgcaagagatatttaataatatagataaaaccgaaactgtctttgttgatatgaatcataaatttcaagcaacccgcatgacatttagcccatggtaa

SEQ ID NO: 11 - protein, 140 aa, Artificial Sequence
  Synthetic
MTYVQRLGTAGERRVMKYLEGTGYKKVFSIQNASGNGLDIVALRPDGKYDIFEVKSSKRGKFKLSERQQKGGKCFAEQVLTEDVTDKGGYNIIDIDGNVKAITSKQARYIYNNIGTTEWVQVNVGRNPKFYDQNITFEEW

SEQ ID NO: 12 - DNA, 423 bp, Artificial Sequence
  Synthetic
atgacctacgtgcagcgcttgggtactgccggcgagcgcagggtaatgaaatatctggaaggtacgggatacaaaaaagtcttctctattcaaaatgcctccggaaacggcttggatattgtcgcattgagaccggatgggaaatatgacatatttgaagttaaaagttctaaacgtggaaaatttaaactaagcgaaagacagcagaaaggcggtaaatgttttgctgagcaagtgttgacggaagatgtaacggataaaggaggttataatattatcgatatagatggtaatgttaaagcaattacaagtaaacaagctagatacatttataataacataggaacaaccgagtgggttcaggtaaatgttggccgaaatcccaaattttatgatcagaatataacgtttgaggaatggtaa

SEQ ID NO: 13 - protein, 1581 aa, Proteus Mirabilis
MGNEFFAAKQGDKLMHSSIFADIVCGVVKGAVYAAIGTAAVALTIGTGGMATAAGVAIFAGAGLAAGFTIGGLIDDAADWVADGIDSLFGLSNPDGEIDTGSKNVRVKGKGAARAAGKLPSPVLLASIAADNTPPKEEKGTEIALRVLKNTAMMATGAFSLLAGKVLGSQTNNAAPIALDPSEFPEYPSPEQGFFDSILSPVKAAKHPNAEPMPLDTITCDKWHCASAPYFLAEGSKKVQINSQPACRNGDRSTCEAKISDTQEKGIVRIGGGSVVVRDIMSGRNPFAEMFGEILGGLAVGAASTLFRRGGLKAIKQCLNSVGCTLVSEIASTAVGSMVVASVTQAFNSVRHPVHAATGAKVLNGEDDTDFIIDGVYPLVWSRIYQSRNTGENRLGRGWSMPFDVFLTIDDTGKGLENENIYYHDMSGRRLALGKIALGQKVFYQDEGFTVYRTTNNLFLVESAEGDYQLFEANPHKINTLRLMKSADRHNNALHYRYANDGELVQIHDDAYLTDIRLHYDEITQRLQSVTRHQGQEEKTLVTYTYDAQQRLVQVTNADKRVTRRFGWDDESGLMAMHQYATGVSSHYRWQRFDAFTIEDNEPEWRVVEHWLKDGKRCLEHTELTYDLAQRTLTTVETGGETTFRRWNEQQQIIEYTNALNETWWFEWDTSRLLTKAIAPDGSEWGYTYDERGNLTQSTDPEQQSTCYDWDKDFAFPTAQTLPNGAAWHWEYNEHGDIRRVIDPLGHITRLAWDDQGLCLGQVDAKGNETHYRYNARGQLIEQRDCSGYPTTLTYDDWGQLRSLTNAQNETTTYTFSEAGLLLTERLPDGTENRYDYDATGQLVGITDAGERHILLRRNRRGQVIARRDPAGHWLHFHYDTFGRMQALENEQGEQYRFEYDALHRLTDEHDLIGQQKHYQYDVMGNVTQIKTTPGPCVDTPMPLSPLVTTFGYDKVGRLLFRENADYRTEYLYQPLSVTLRRVPMAIWHEAERTGTTARVEYQDALTFTYDKVGQLVREASARDDYQHHYDVLGNITRTELPHQRAFEYLYYGSGHLQQTQWRDNAQLTVLAEYQRDRLHRETLRTSGALDNETGYDCRGRITHQVARQMNASQFVTPVIDRRYRWDKRNQLIERSVSYGQTGEVFTAGHWYYHSYQYDPLGQLTAHLGSVQTEHFLYDAAANLLTRPHSEAPHNQVQGSDKYDYRYDGFGRMVSRYEKGSSSGQRYHYDSDHRIIAVDIDQGPLGYQRAEYRYDILGRRIEKRLWKASAIANTVTYHQHEPDEVYTFGWVGMRLVSEHSSAAPHTTVYHAYNDQSYTPLARIECTDNPLNPQRAIYYTHSSLSGLPEALTNSEGEIVWQGQYSVWGHLQRQTRPTSTFNREQNLRFQGQYFDKETGLHYNTFRYYAPDLGRFTQQDPIGLAGGINLYAYAPNPLTWVDPWGWSCFSARVGAFGEKRVMKYLSGAGYKKVFSVQNNSGHGLDIVALRPDGKFDIFEVKSSTIGQFSLSSRQATGDDFAKIVLLNDVKKGGYNIIDIDGNVKAITSKQARYIYNNIGTTEWVQVNVGRNPKFYDQNITFEEW

SEQ ID NO: 14 - DNA, 4746 bp, Proteus Mirabilis
atgggaaatgaatttttcgcagccaagcaaggcgataaattaatgcactcgtcaatcttcgccgatatcgtgtgtggtgtggttaaaggggcggtttatgccgcgatagggacggccgctgtggcgttgactattggtacaggtggtatggcaacggccgccggtgttgccatttttgccggtgcagggctggcggcgggctttaccatcggtggattgattgatgatgccgctgattgggtcgcggatgggatcgatagcttgttcgggctaagtaaccctgatggtgaaatcgatactggctcgaaaaatgtacgtgtcaaaggcaagggagccgctcgcgcagcgggaaaattaccgtcacccgtactattagcctcaattgcggcagataataccccaccgaaagaagaaaaaggcaccgagattgccttacgcgtattgaaaaataccgccatgatggcgactggggcattttctttattggctggtaaggtgctagggagccaaaccaacaatgcggcacccatagcgctcgatccctccgagtttcccgaatacccctcaccggagcaaggtttttttgacagtattctttccccagtaaaagctgcgaaacacccgaatgctgaacctatgcctttggataccatcacgtgcgataaatggcattgtgctagcgccccttactttttagccgaagggtcgaaaaaagtccaaattaatagccaaccggcgtgtcgtaacggtgatagaagcacttgtgaagccaaaatcagtgatacgcaagaaaaagggattgtgcgtattggcggtggcagtgtggtggtacgcgatattatgagtgggcgtaacccctttgccgaaatgtttggtgaaattctcggtggccttgctgttggagcggcgtctaccctgtttcgcagagggggactcaaggcgattaaacagtgtttgaatagtgtaggctgtactttggtgagtgaaatcgccagtaccgcggtgggcagtatggtagtcgcctcggtgacccaagcctttaatagtgtgcgtcaccccgtgcatgcggctacgggggcgaaagtcctaaatggagaggatgataccgattttattattgatggtgtgtacccgttagtctggtcgcgtatttaccaaagtcgtaatacgggtgaaaatcgtttaggacgcggttggtcgatgccgtttgatgtctttttgacaattgacgacacaggtaaagggcttgagaatgaaaacatttattatcatgacatgtcggggaggcgtttagcgttaggaaaaatcgcccttggacagaaagtgttctatcaagatgaaggctttaccgtctatcgcacgacaaacaatcttttccttgttgaatcggcggaaggtgattatcaacttttcgaagccaatccacataaaatcaatacactgcgcttaatgaaaagtgctgatcgtcataataacgctttacactaccgctatgctaatgacggtgaattggtacaaattcatgatgatgcgtatctgaccgatatccggttacattatgatgaaatcacccaacgcttacagtcggtgacccgccaccaaggacaagaagaaaaaacgctggtcacttatacttatgatgcacaacagcgtttagtgcaagtcactaatgcggataagcgagtgactcgtcgttttggctgggatgatgaatcgggtttaatggccatgcaccaatatgccacaggggtcagtagtcattatcgttggcaacgttttgatgcctttaccattgaagacaatgaacctgagtggcgagtggttgaacactggcttaaagacggtaaacgctgtttagagcataccgaactgacgtatgatttagctcaacgaaccctcaccaccgtggaaacggggggtgaaaccaccttccgtcgctggaatgaacaacagcaaattatcgaatacaccaatgcattgaatgaaacgtggtggtttgaatgggataccagccgcttattaacgaaagcaatagccccagatggcagtgaatgggggtatacctatgatgagcgcggtaatttaacccaatcgaccgatccggaacaacaatcgacttgctatgattgggacaaagattttgcgtttcccacggcacaaactttgccaaatggggcggcttggcattgggagtacaatgagcacggcgatattcgtcgtgtgattgacccgcttggccatatcacgcgcttggcgtgggatgaccaaggcctgtgtctggggcaagtggatgctaagggtaatgaaacccactatcgctataatgcccgcggtcagttaatcgagcaacgggactgttcgggttatcccaccaccttgacctacgatgattggggacaacttcgctcgttgaccaatgcacagaatgaaaccacgacttacacctttagtgaagcggggttgttgttaacagagcgcttaccggatgggacagaaaaccgttatgactatgatgccaccgggcaattagtgggaataacggatgccggagagcgccatattctgttgcgccgtaaccgtcgtggacaagtgatagcccgacgcgatccggcagggcattggttgcattttcactatgatactttcgggcgcatgcaggcactggagaatgaacagggcgagcagtaccgctttgagtatgatgccttgcatcgtttaaccgatgaacatgatcttatcggacaacaaaagcactatcagtacgatgtgatggggaatgtcacccagataaaaactaccccggggccttgcgttgatacgccgatgcccttatccccgctggtgaccaccttcggttacgataaagtgggacggttgctgtttcgagaaaacgccgattatcgcacggaatacctttatcaacctttgagtgtgacgttacgccgagtccccatggcgatatggcatgaagccgaacgcaccggcaccaccgcccgcgtagagtatcaagacgccctgacctttacctacgataaagtggggcagttggttcgagaagccagtgcccgcgatgattatcagcatcactatgatgtgctgggtaatatcactcgcaccgagttgccccatcaaagggcatttgaatacctgtattacggctctgggcatttacaacaaacgcaatggcgagataatgcgcaactgacggtattggcggaatatcaacgagaccgtttacaccgagaaacgttgcgcaccagtggcgccttagacaatgaaacgggctatgactgtcggggacgtattacgcaccaagtggcgcgccaaatgaatgcctcacagtttgtgaccccggtgattgaccgtcgttatcgttgggataaacgcaatcaacttatcgagcgcagtgtcagttatggtcaaacaggcgaggtttttaccgccggacattggtactaccacagttatcagtacgacccgctggggcaattgacggcgcatttagggtcggtgcaaaccgaacactttctgtatgatgcggcggccaatttactgactcgtccgcacagcgaagcaccgcataaccaagtacaaggcagtgataagtatgattatcgctacgacggttttggtcgcatggtctcccgctatgaaaaaggcagtagctcgggacaacgttatcactatgacagtgaccaccgcattatcgcggtggatatcgaccaagggcccttagggtatcagcgagcggaataccgctacgatattttagggcgccggattgaaaaacggttatggaaagccagtgcgattgccaacacagtcacctatcaccaacatgagccggatgaagtgtacacttttggctgggtagggatgcgactggtgtctgagcacagcagcgcagcgccccataccacggtttatcatgcatacaatgaccaaagttatacgcctcttgctcgcattgaatgcacggataacccgcttaatccgcaacgggcgatttattatacgcacagcagtttaagtggcttaccggaagcgttgaccaacagtgagggtgaaatagtttggcaagggcaatacagtgtttggggacatttacagcgtcaaacccgccccacaagcacatttaatcgtgaacagaacctgcgctttcaagggcagtatttcgacaaagagacggggttacattataatacgttcaggtactatgccccggatttaggccgttttacccagcaagacccaatagggttggcagggggcataaacttatatgcttatgcgccaaatccattgacatgggtggatccgtggggttggagttgctttagtgctcgggtaggtgcttttggtgagaaaagagttatgaaatacttatctggagcgggctataaaaaagttttttctgtacaaaacaattctgggcatggtctggatatagttgctttaagaccagatggaaaatttgatatttttgaagttaaaagttcgacaataggacaattttctttatcttcccgccaagctacaggcgatgattttgcaaaaatagttcttttaaacgatgtgaaaaaaggaggttataatattatcgatatagatggtaatgttaaagcaattacaagtaaacaagctagatacatttataataacataggaacaaccgagtgggttcaggtaaatgttggccgaaatcccaaattttatgatcagaatataacgtttgaggaatggtag

SEQ ID NO: 15 - protein, 8 aa, Artificial Sequence
  Synthetic
DYKDDDDK

SEQ ID NO: 16 - DNA, 24 bp, Artificial Sequence
  Synthetic
gactacaaggacgacgatgacaag

SEQ ID NO: 17 - DNA, 19 bp, Artificial Sequence
  Synthetic
gctagccatttgcccatgg

SEQ ID NO: 18 - DNA, 27 bp, Artificial Sequence
  Synthetic
cgtttttgataaaaggatattgttgag

SEQ ID NO: 19 - DNA, 757 bp, Artificial Sequence
  Synthetic
tcgcccacccccatgggcaaatggctagcttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcgttgatagagttattttaccactccctatcagtgatagagaaagtttttgataaaaggatattgttgagcac

SEQ ID NO: 20 - DNA, 20 bp, Artificial Sequence
  Synthetic
gtcaaggagctctcatgtgc

SEQ ID NO: 21 - DNA, 37 bp, Artificial Sequence
  Synthetic
caataaaccggtctaccattcctcaaacgttatattc

SEQ ID NO: 22 - DNA, 38 bp, Artificial Sequence
  Synthetic
gtacatgagctctcatgagtaaaggagaagaacttttc

SEQ ID NO: 23 - DNA, 36 bp, Artificial Sequence
  Synthetic
caataaaccggtctatttgtatagttcatccatgcc

SEQ ID NO: 24 - protein, 4 aa, Artificial Sequence
  Synthetic
PAAR

SEQ ID NO: 25 - protein, 82 aa, Proteus Mirabilis
CFSARVGAFGEKRVMKYLSGAGYKKVFSVQNNSGHGLDIVALRPDGKFDIFEVKSSTIGQFSLSSRQATGDDFAKIVLLNDV

SEQ ID NO: 26 - protein, 77 aa, Rothia Aeria
SFSQAVGNAGESQLLRYLNLKETGYKEVFAVQNASGNGLDAVARRPDGKYDIFEVKSSTVGKFELSDRQAKGGKGFA

SEQ ID NO: 27 - protein, 84 aa, Rothia Aeria
TYVQRLGTAGERRVMKYLEGTGYKKVFSIQNASGNGLDIVALRPDGKYDIFEVKSSKRGKFKLSERQQKGGKCFAEQVLTEDVT

SEQ ID NO: 28 - protein, 70 aa, Rothia Aeria
MKYLEGTGRYKKVSSIQNASGNGLDIVALRLDGKYDIFEVKSSKRGNFRLSERQQKGGKCFAEQVLMKDV

SEQ ID NO: 29 - protein, 70 aa, Unknown; identical to SEQ ID NO: 28
  Rothiasp. Olga

SEQ ID NO: 30 - protein, 55 aa, Proteus Mirabilis
KKGGYNIIDIDGNVKAITSKQARYIYNNIGTTEWVQVNVGRNPKFYDQNITFEEW

SEQ ID NO: 31 - protein, 5 aa, Rothia Aeria
ELFFN

SEQ ID NO: 32 - protein, 55 aa, Rothia Aeria
DKKKGGYFMKGLDGKKTPLNKKKAQEIFNNIDKTETVFVDMNHKFQATRMTFSPW

SEQ ID NO: 33 - protein, 53 aa, Rothia Aeria
KKGGYFMKGLDGKETPIGPKEAQEIFNNIDKTETVFVDMNSKFRATRITFGLW

SEQ ID NO: 34 - protein, 53 aa, Unknown; identical to SEQ ID NO: 33
  Rothiasp. Olga

SEQ ID NO: 35 - protein, 137 aa, Proteus Mirabilis
CFSARVGAFGEKRVMKYLSGAGYKKVFSVQNNSGHGLDIVALRPDGKFDIFEVKSSTIGQFSLSSRQATGDDFAKIVLLNDVKKGGYNIIDIDGNVKAITSKQARYIYNNIGTTEWVQVNVGRNPKFYDQNITFEEW

SEQ ID NO: 36 - protein, 128 aa, Prevotella Jejuni
RPFGHAVGDIGEKAVINDLKKNNYEIIDVKYGSNNGIDVLAKNPSGKYDAFEVKSSTVGKFNLSEAQMDPDNFVRTRLERAESHGMITEDARIDIMSNLGERKVAYVDIKRRDNGALYAESIHYESWD

SEQ ID NO: 37 - protein, 128 aa, Prevotella Denticola
RPFGHAVGDIGEKAVINDLKKNGYEIIDVKYGSNNGIDVLAKNPNGKYDAFEVKSSTVGNFNLSEAQMNPSDFVKTRLNEAELRGKINEDTRAEIMSKLGERKIAYVDIKRGKKGKLYADSISYENWD

SEQ ID NO: 38 - protein, 128 aa, Unknown
  Prevotella sp. C561
RPFGHAVGDIGEKAVINDLKKNNYEIIDVKYGSNNGIDVLAKNPSGKYDAFEVKSSTVGNFNLSKAQLKPENFVKTRVNNAVANGKINKRTRRDIMTNLGDRKVAVVGIKRGEKGKLYADSIRYENWD

SEQ ID NO: 39 - protein, 128 aa, Prevotella Fusca
RPFGHAVGDIGEKAVINDLKKNGYEIIDVKYGSNNGIDVLAKSPSGKYDAFEVKSSTVGKYNLSEAQMDPENFVKTRVNNAVANGKINKRTRRDIMTNLGDRKVAVVGIKRGEKGKLYADSIRYENWD

SEQ ID NO: 40 - protein, 130 aa, Cronobacter Turicensis
CFSKRLGDFGESHVKSQLERSGYADVFSVQNKSNNGIDLVGLRHDGKYDFFEVKTNTTGSVKLSDRQLSSKDFIETVLGTDVRKGGYDLNGHRVNDMLDNIGDTYVVDVFVERASNGRWFVSKYLMSIWE

SEQ ID NO: 41 - protein, 130 aa, Cronobacter Sakazakii
CFSKRLGDFGESHAQSQLKASGYADVFSVQNKSNNGIDLVGLRHDGKYDFFEVKTNTTGSVKLSDRQLSSKEFIETVLRNDVPKGGYDLNGHVVDEMLDNIGDTYVIDVFVGRASNGRWFVSKYLMSIWE

SEQ ID NO: 42 - protein, 128 aa, Algibacter
PNFGVKMGDWGEKVASKYLSKNGHQVLGSVQNASGHGFDLITKTSDGNINVIEVKTSKSNWSKADMSGWTDRNIRKISGNINGRWKNMPNYQKNLMDIIIDAKQWGKLKNKLLQINIDERSIKLKCKS

SEQ ID NO: 43 - protein, 125 aa, Taylorella Asinigenitalis
WSAIGPWGEKIASKFLNMQGHKILGSIQNASGHGFDLVTMTPDGFINIIEVKTSQSYWSKSNMSTWTNNNIRKMMGNTNGRWGNMPNYQKNLLRNIRDAQAKNKLRNILVQINIDERSIRIQCKG

SEQ ID NO: 44 - protein, 135 aa, Rothia Aeria
TYVQRLGTAGERRVMKYLEGTGYKKVFSIQNASGNGLDIVALRPDGKYDIFEVKSSKRGKFKLSERQQKGGKFAEQVLTEDVKKGGYFMKGLDGKKTPLNKKKAQEIFNNIDKTETVFVDMNHKFQATRMTFSPW

SEQ ID NO: 45 - protein, 128 aa, Porphyromonas Macacae
RSFTQAVGDIGEKYYTGVLKTQGFKIIDVKYGSNWGIDILAKNPQGKYDFFEVKSSTVGSFNLSPDQMSSKEFVQTRLLSAREKGVISPDLYDDIVANMGERKLVNVDISRADNGKLYASNAEIKDWH

SEQ ID NO: 46 - protein, 135 aa, Acinetobacter Baumannii
QNFDPSLGTWGEETAVQLVKGSGFTDVISIQNASGNGIDIIAKNPQGNYVYLEVKTSAVGKISLSKRQEDMNFFVNDILTNAAKTGRYVNISDTVNAQAKIMLTKYKQQKAVGNIYGAAIGVDLKNAQILISSWT

SEQ ID NO: 47 - protein, 135 aa, Acinetobacter Baumannii
QNFDPSLGTWGEETAVQLVKGSGFTDVISIQNASGNGIDIIAKNPQGNYVYLEVKTSAVGKISLSKRQEDMNFFVNDILTNAAKTGRYINISDTVNAQAKIMLTKYKQQKAVGNIYGAAIGVDLKNAQILISSWT

SEQ ID NO: 48 - protein, 10 aa, Artificial Sequence
  Synthetic
HHHHHHHHHH

SEQ ID NO: 49 - protein, 10 aa, Artificial Sequence
  Synthetic
EEEEEEEEEE